(19) United States
(12) Patent Application Publication
(10) Pub. No.: US 2010/0286247 A1
(43) Pub. Date: Nov. 11, 2010

(54) Methods of Protection from Oxidative Stress

(75) Inventors: Barbara A. Gilchrest, Boston, MA (US); Mark S. Eller, Boston, MA (US); Mina Yaar, Sharon, MA (US); Margaret S. Lee-Bellantoni, Brookline, MA (US)

Correspondence Address: HOWREY LLP - East, C/O IP DOCKETING DEPARTMENT, 2941 FAIRVIEW PARK DR, SUITE 200, FALLS CHURCH, VA, 22042-2924, US

(73) Assignee: Trustees of Boston University, Boston, MA (US)

(21) Appl. No.: 11/909,435

(22) PCT Filed: Apr. 4, 2006
(86) PCT No.: PCT/US06/12468

§ 371 Date: Jul. 2, 2010

Related U.S. Application Data
(60) Provisional application No. 60/668,288, filed on Apr. 4, 2005.

(51) Int. Cl. A61K 31/7088; A61P 3/10; A61P 9/00; A61P 9/12; A61P 17/00; A61P 25/28; A61P 27/02

(52) U.S. Cl. 514/44 R

(57) ABSTRACT

Alterations in the structure of telomeres lead to modulation in the redox state of the cell. Substances which mimic destabilized telomeres, such as t-oligos, have a protective effect on future exposure of a cell to oxidative stress.

### PRIORITY CLAIM

This application claims priority from U.S. Provisional Patent Application 60/668,288 filed on Apr. 4, 2005, the entire disclosure of which herein is incorporated by reference.

### FIELD OF THE INVENTION

The invention is related to a method of reducing the risk of an oxidative stress-related event.

### BACKGROUND OF THE INVENTION

The Free Radical Theory Of Aging Meets The Telomeric Biological Clock Theory The study of reactive oxygen species (ROS) and oxidative stress in fibroblast biology is important in the context of multiple cellular phenomena, including senescence at the cellular level and aging of organisms. Aging has been described as cellular attrition and senescence eventually leading to decreased viability and death, influenced by genetic program as well as by cumulative environmental and endogenous insults. It is thought that intrinsic aging involves genetically predetermined internal changes, such as telomere shortening, progressive downregulation of hormone production or repair systems, or is due to an excess of toxic metabolic byproducts. Extrinsic aging can be described as progressive dysfunction due to damage incurred from external sources such as toxins, radiation, and infections.¹ The “free radical theory of aging” proposes that cells and organisms will eventually die due to progressive damage incurred at least in part by ROS.^{2,3} However, it is now accepted that ROS are actively produced and utilized by cells also, as a mechanism of signal transduction, and it is unclear whether this simultaneously creates oxidative damage.⁴ This is important in the study of DNA damage and lifespan because it argues that through evolution organisms may have learned to actively modulate and utilize ROS while responding to changing redox states and preventing oxidative damage. Aerobic organisms have evolved to utilize oxygen for energy metabolism, but an excess of ROS has been implicated in numerous disease states such as atherosclerosis,⁵ allergy,⁶ cancer,⁴ neurodegenerative disorders,⁷ scleroderma⁸ and premature aging syndromes,^{9,10} suggesting that ROS homeostasis and the recognition and repair of oxidative damage is essential for health and longevity.⁴ It is now thought that cells actively enter different physiologic states (repair, growth arrest, senescence or apoptosis) depending on the oxidative stimuli.¹¹ The ability to respond in different ways to oxidative damage may be crucial for avoiding carcinogenic transformation and maintaining the health and life of multicellular organisms.

The current investigation combines recent knowledge of oxidative stress and ROS signaling with the understanding that telomeres are sensitive to oxidative damage.¹² Telomeres are DNA structures at the ends of chromosomes that are thought to both physically protect the ends of chromosomes and, more recently, to participate in regulatory pathways in the nucleus.¹³ Since Hayflick reported in 1961 that normal human fetal fibroblasts undergo a finite, predictable number of population doublings in culture,¹⁴ his suggestion that there must be a counting mechanism to meter the number of cell doublings has been supported by our knowledge of telomeric structure and function in cells. The “telomere hypothesis of aging” links telomere length to replicative potential and lifespan. Growing evidence suggests that integrity of the three-dimensional looped structure at the distal portion of telomeres is as essential for proper telomere function as telomere length, and that the telomere loop structure is constitutively monitored by the cell.¹⁵ Telomeric DNA damage, which includes oxidative base modification,¹⁶ is likely to involve telomere loop disruption¹⁷ that triggers signaling cascades and adaptive antioxidant responses. What these antioxidant responses might be has not been characterized. We utilized telomere homolog oligonucleotides that mimic telomere loop disruption to study oxidative telomere damage responses.

The material below further reviews general background information, introducing the concept of mimicking telomere damage and inducing responses using the thymidine dinucleotide pTT and an 11-base sequence pGTTAGGGTTAG (SEQ ID NO: 1) (abbreviated here as TO) that is fully homologous to the telomeric single-stranded 3′ overhang region.

### Telomeres

### Telomere Structure

Telomeres were first identified in the late 1930's as DNA structures at the ends of chromosomes,¹⁸ and little was known about their function. They were thought to protect the chromosome ends to prevent end-to-end fusion or to facilitate attachment of the chromosome to the nuclear envelope.¹ In the 1970's telomeres were found to consist of hexameric nucleotide repeat sequences, in the protozoan Tetrahymena, as TTGGGG.¹⁹ This G-rich strand is paired to its complementary strand except at the most distal 6-12 bases, forming a 3′ overhang that in vitro was reported to form hairpin loops of duplex telomeric DNA stabilized by hydrogen bonds.²⁰ Tetrahymena telomere sequences in solution also form antiparallel guanine base tetrads between two hairpin loops, raising the possibility that even more complex telomeric structures exist.²¹

In 1988, mammalian telomeres were reported to consist of multiple tandem repeat sequences of TTAGGG at the 3′ ends of chromosomes,²² and in 1997 reported to have a conserved G-rich 3′ overhang much larger than is found in protozoans, on the order of 50-150 bases long.²³ In 1999, Griffith et al. provided electron microscopic evidence that protection of the overhang involves a loop configuration they named a “t loop.”²⁴ The size of the t loop is proportional to the number of nucleotide base pairs in the entire telomere structure.²⁴ Previously, electron microscopy had also shown that telomeres are tightly compact;²⁵ together this data suggests a high degree of tertiary telomere structuring.

Telomere binding proteins, named telomere repeat factors 1 and 2 (TRF1 and TRF2), were identified and reported to contribute to formation and stabilization of the t loop by binding to duplex telomeric DNA on the G-rich strand.^{26,27} The G-rich 3′ single-stranded overhang is thought to be shielded and secured within DNA-protein complexes comprising the proximal duplex telomeric DNA and TRF2, named a “d loop.” TRF2 was found to bind at the junction of duplex DNA and the 3′ overhang, requiring at least six unpaired nucleotides of the overhang for loop formation.²⁸ More recently, a protein called Pot1 (protection of telomeres) was also found to bind to single-stranded telomeric DNA and is thought to cooperate with TRF2 in maintaining the d loop structure.²⁹⁻³¹ See FIG. 1 for a diagram of the proposed telomere loop structure (chromosomes end with telomeres, which contain single-stranded DNA that is looped and secured by several proteins, including TRF 1, TRF2 and Pot1, into the proximal double-stranded telomere region (at the d loop) to form a physical cap called a t loop. The single-stranded 3′ overhang sequence in human telomeres consists of tandem repeats of TTAGGG).

A complex of additional proteins associated with DNA damage and repair, RAD50/MRE11/NBS1, were found to associate with the telomeric DNA-TRF2 complex only during S-phase, possibly to modulate t loop stability during DNA replication.³² This suggests a link between DNA damage repair, telomere maintenance and cellular proliferate potential.

### Telomere Function

Muller identified and named the telomere in 1938, and predicted that telomeres serve to physically protect the ends of chromosomes.¹⁸ The t loop configuration is thought to shield the overhang DNA, preventing its modification and degradation by ligases and nucleases.²⁷ Without this stabilization and protection of the overhang, accelerated telomeric shortening occurs, resulting in telomere dysfunction and leading to chromosomal instability, end-to-end fusion of chromosomes, and/or apoptosis.^{27,33-36}

It is also thought that the multiple tandem repeats in telomeres may serve as a “buffer zone” for DNA polymerase, which cannot fully replicate the 3′ end of duplex DNA due to the physical limitation of the enzyme in simultaneously binding and replicating the same section of DNA. This is known as the “end replication problem.”^{37,38} Telomeres provide additional substrate for DNA polymerase to anchor onto, enabling the cell to replicate all crucial information even though a portion at the end of the telomere is progressively lost during each round of replication.

### Maintaining Telomere Length

It was discovered in the mid-1980's, in Tetrahymena, that the length of telomeres is regulated by a ribonucleoprotein enzyme complex that was named telomerase.³⁹ There are at least three major components to the enzyme complex: a telomerase reverse transcriptase (TERT) catalytic subunit, an RNA template (TR), and a telomerase-associated protein (TP1).⁴⁰⁻⁴² Telomerase activity can be detected using a PCR-based “telomere repeat amplification protocol” (TRAP) in most cancers and in normal human cells that either rapidly proliferate (fetal tissue, peripheral blood lymphocytes, intestinal crypt cells, and basal skin epidermis), or have the potential to give rise to many cells (marrow stem cells and germ cells). Telomerase was not thought to be active in most other somatic cells.⁴³ However, there is now evidence that telomerase may be expressed transiently in other cells and tissues, such as in fibroblasts at wound edges.^{44,45}

### Telomeres as a Biological Clock

Telomeres were first linked to aging when it was found that telomeres shorten progressively with DNA replication and critically short telomeres were associated with senescence in many cell types.^{46,47} In 1961 Hayflick observed that fibroblasts achieve a finite number of cell doublings (40-60 doublings) before reaching senescence, which is an irreversible nonreplicative state.¹⁴ This finite number of replicative doublings is known as the “Hayflick limit.”¹⁴ He also reported that these fibroblasts retain a “memory” of doubling frequency even through freezing and re-culturing, and telomere shortening offers a mechanistic explanation for this phenomenon. Cells cultured from frozen stock proliferated only until the total of pre-freeze and post-freeze doubling equaled the Hayflick limit.¹ One can infer that together with DNA replication and all other cellular functions, progressive telomere shortening stops during cryogenic storage, and this resumes upon thawing and re-culturing.

A landmark paper by Bodnar et al. in 1998 firmly established the telomere hypothesis of aging by showing that transfecting telomerase into retinal epithelial cells and skin fibroblasts caused them to exceed the Hayflick limit, maintain long telomeres, and display reduced senescence-associated-β-galactosidase staining.⁴⁸ Shorter telomeres and decreased replicative potential are found in cells from patients with the premature aging syndromes Hutchinson-Gilford progeria and Werner syndrome, and from telomerase RNA null (mTR−/−) mice, which also display a premature aging phenotype.^{49,50 51} These are excellent models for studying the role of telomeres in aging, although to date it remains unclear precisely how telomere length and its regulation serve as a “biologic clock.”

The complexity of telomere regulation is reflected by the many contradictory findings regarding the relationship between telomerase activity, telomere length and cell lifespan in vitro, as well as in cloning studies in vivo.^{48,52-62} For example, some cancers were found to have shorter telomeres than those reported in their normal counterparts.^{52-54,63} Clones of fibroblasts expressing the catalytic component of telomerase (TERT) do not senesce even when the telomerase is inhibited by a dominant negative mutant form, causing the cells to develop very short telomeres.⁵⁶ In mice lacking the gene encoding the telomerase RNA subunit, scientists were still able to create cell lines, achieve viral oncogenic transformation and stimulate tumor formation.⁵⁷ Bovine calves cloned from senescent cells by Lanza et al. displayed longer telomeres than those of age-matched controls,⁶¹ but Dolly, the first cloned animal (also from senescent donor cells), had short telomeres and died at half a normal sheep's lifespan.⁶² Furthermore, about a third of immortalized human cell lines in vitro have no detectable telomerase, yet have abnormally long telomeres. These cells are said to have an alternative telomere maintenance mechanism (ALT, Alternative Lengthening of Telomeres), which is still poorly understood, but seemingly independent of human telomerase gene expression and function.^{64,65}

### T Loop Disruption and Cellular Senescence

Increasing evidence supports the concept that the key signal for senescence is disruption and exposure of the telomeric single-stranded 3′ overhang.⁶⁶⁻⁶⁸ Van Steensel et al. and Smogorzewska et al. showed that increased expression of the telomere binding proteins TRF1 and TRF2 results in shortened but stable telomeres, possibly due to increased sequestration of the 3′ terminus from telomerase.^{69,70} It was also concluded that neither protein regulates telomerase activity directly.^{69,70} A TRF2 dominant negative protein disrupts loop formation and activates the tumor suppressors ATM and p53, which then stimulate DNA damage responses such as cell cycle arrest and apoptosis.^{32,71} Later, it was discovered that overexpression of TRF2 accelerated telomere shortening and yielded, abnormally short telomeres, yet delayed senescence, emphasizing the importance of telomere structure over telomere length.⁶⁷ Saretzki et al. demonstrated induction of p53, cyclin-dependent kinase p21, and cell cycle arrest in fibroblasts and glioblastoma cells treated with oligonucleotides with a (TTAGGG)₂ sequence.

In recent experiments using normal human dermal fibroblasts, prolonged treatment with the T-oligo pGTTAGGGTTAG (TO, SEQ ID NO: 1) for 7 days induced several markers of senescence.^{68,72} Li et al. observed induction of p53, p21, and p16^{INK4a}; hypophosphorylation of retinoblastoma protein pRb; expression of senescence-associated-β-galactosidase in over 60% of TO-treated cells as compared to controls; and the formation of enlarged, flattened, senescent cell morphology of the β-galactosidase positive cells as a response to mimicked telomere damage.^{68,72} FIG. 2 summarizes reported signaling responses, including senescence, observed after modeling t loop disruption with various T-oligos (considerable evidence supports that telomere loop disruption in the key event triggering multiple DNA damage responses. Shown is a summary of ways to disrupt the t loop or mimic t loop exposure and the resulting signaling and adaptive responses published to date. Rectangles highlight findings using mimicked t loop disruption using (TTAGGG)ₙ oligonucleotides. Oval highlight findings using pTT or TO, which overlap with the other findings.)

### Cellular Oxidative Stress

Cellular oxidative stress was defined by Helmut Seis in 1985 as “a disturbance in the prooxidant-antioxidant balance in favor of the former.”⁷³ In short, the redox status of a cell in oxidative stress promotes oxidation reactions (a gain in oxygen or loss of electrons) over reduction (loss of oxygen or gain of electrons).⁷⁴ Oxidative stress is harmful to cells because oxidative modification of lipids, carbohydrates and DNA can impair normal function and even accelerate senescence.⁷⁵ It is a constant potential danger because oxygen is prevalent in the internal environment of the cell, and mitochondria generate reactive oxygen species (ROS), reactive metabolites of oxygen including free radicals, in the electron transport chain, although it is not known for certain whether they consistently contribute to oxidative stress in the entire cell.⁷⁶

Free radicals are defined as any atoms or molecules with one or more unpaired electrons in their outer orbitals.⁷⁷ Many metabolites of oxygen are termed ROS because they are more reactive relative to oxygen (O₂), and in addition to free radicals, include molecules that do not meet the definition of a radical. Examples of biologically important ROS are superoxide anion (O₂.⁻), hydrogen peroxide (H₂O₂), hydroxyl radical (OH.), singlet oxygen (¹O₂), nitric oxide (NO.) and peroxynitrite (ONOO—).^{77,78} OH. is so reactive that it can modify any DNA or RNA base or sugar and create single and double strand breaks. ¹O₂ has been found to predominantly modify guanine bases, yielding 8-oxo-7,8-dihydro-2′-deoxyguanosine (8-oxo-dG).⁷⁹ O₂.⁻, H₂O₂ and NO. do not directly damage DNA; however, they may promote DNA damage by contributing to the formation of the more reactive species.⁸⁰

### The Free Radical Theory of Aging

The free radical theory of aging was proposed by Harman in 1956 when he observed that aging and damage due to ionizing radiation are both characterized by cellular dysfunction, increased mutagenesis and carcinogenesis.⁸¹ The free radical theory of aging later incorporated the concept of mitochondrial oxidative metabolism as central to the aging process, not only because mitochondria generate ROS,⁸² but also because they themselves are targets for oxidative damage.⁸³ The sites and degree of ROS production and damage in mitochondria are still the subject of much investigation and speculation,^{76,83} but progressive accumulation of oxidative damage in mitochondrial DNA (mtDNA) is suggested to be a primary cause of aging and death.^{81,84} This theory has drawn attention to the potential protective role of antioxidants such as α-tocopherol (vitamin E), ascorbic acid (vitamin C) and antioxidant enzymes, especially in the mitochondria.^{85,86} Free radical scavengers and antioxidant enzymes protect cells by reacting with damaging free radicals and ROS before they can oxidize and damage important cellular structures and molecules such as DNA. Mitochondria, when damaged, are also important participants in apoptosis;⁸⁷ therefore, it is reasonable to conclude that preserving mitochondrial function through adequate antioxidant defense is an important determinant of a cell's or organism's viability.

Oxidative damage has also been implicated in carcinogenesis, due to intracellular sources of oxidative stress as well as environmental effects such as ultraviolet A (UVA) radiation (320-400 nm), which can generate ROS via excitation of endogenous chromophores.^{88,89} Much current research addresses the effects of oxidative stress upon DNA, mitochondrial function, antioxidant defense, and cell senescence or aging. It is accepted that antioxidant molecules and antioxidant enzymes are protective against disease and cellular degeneration, but much remains to be elucidated. For example, mechanisms of antioxidant enzyme control and oxidative mtDNA damage and repair are still being studied, and the degree of contribution of different wavelengths of UV to carcinogenesis through ROS generation is still under investigation.^{75,83,88-90}

### Telomeres and Oxidative Stress

There is evidence that telomeres are more susceptible to oxidative damage than the rest of the genome, at least in part due to the high percentage of guanine bases in the telomere sequence.⁹¹ As mentioned above, guanosine nucleotides are known to undergo oxidative base modification, yielding 8-oxo-dG, a common biomarker for oxidative stress and oxidative DNA damage.⁹⁰ Guanines are one of the main oxidative targets for singlet oxygen,⁹² which can be generated by excitation of oxygen through endogenous cellular chromophores such as porphyrins following UV or visible light exposure.⁹³ Telomeric sequences have been shown to yield more 8-oxo-dG than nontelomeric sequences in a cell-free system containing H₂O₂ and Cu(II), which generates DNA-damaging hydroxyl radicals.⁹⁴ Von Zglinicki et al. found that fibroblasts exposed to chronic hyperoxia display accelerated aging and shortening of telomeres,¹² which may be explained by their subsequent finding that oxidative stress created single-strand breaks in telomeres that were not repaired as efficiently as they were repaired in the bulk of the genome.⁹¹ They showed that hyperoxia leads to induction of p53, p21 and cell cycle arrest, and stimulated the same responses by treating cells with telomeric oligonucleotides (TTAGGG)₂, leading them to conclude that oxidative stress leads to the production of G-rich single stranded oligonucleotides during the process of telomere shortening, and that these fragments of telomeric DNA trigger p53-dependent cell cycle arrest.⁹⁵ Furumoto et al. were able to counter shortening of telomeres caused by hyperoxia by treating cells with an oxidation-resistant derivative of ascorbic acid, Asc-2-O-phosphate (Asc2P).⁶⁶ Also, it was very recently shown that oxidative modification of even one telomeric guanine base to form 8-oxo-dG, or the presence of base excision repair (BER) intermediates, causes TRF1 and TRF2 binding to decrease by 50% or more.¹⁷ This decreased binding could lead to t loop opening and the observed telomere shortening during oxidative stress.^{17,70} Data suggests that such exposed telomere ends become vulnerable to inappropriate nuclease modification and DNA ligase-mediated chromosome end-joining²⁴ as well as to exogenous causes of DNA damage such as radiation⁹⁷ or further sensitivity to endogenous ROS.⁹⁸ It has thus been proposed that telomere t loop protection against oxidative damage helps prevent early senescence.^{99,100} (see FIG. 3).

### Antioxidant Defense

Defense against oxidative DNA damage requires antioxidant molecules and enzymes. Halliwell and Gutteridge have defined antioxidants as “any substance that, when present at low concentrations compared with those of an oxidizable substrate, significantly delays or prevents oxidation of that substrate.”¹⁰¹ DNA has the added protection of the BER pathway, which specifically repairs oxidized DNA bases such as 8-oxo-dG.^{16,90}

### Antioxidant Molecules and Enzymes

There are many families of antioxidant molecules with various structures and mechanisms of antioxidant action. These include selenoproteins (including the major antioxidant protein glutathione), plant phenols (such as flavonoids, containing a characteristic 3-ring structure), carotenoids (such as β-carotene and lycopene), thiols (such as the chemical N-acetylcysteine), iron regulation proteins or chelators, and other substances commonly found in plants and fruits.^{102,103}

Antioxidant enzymes (AOE) are a major source of protection because they are expressed abundantly and constitutively, and are inducible.^{6,104} FIG. 4 shows the relationship between some of the major ROS studied in skin and major enzyme reactions. Although there are many antioxidant enzymes and isoforms within the same family of enzymes, the major AOE in human tissues that are best understood are the superoxide dismutases, catalase, and glutathione peroxidase. Copper-zinc superoxide dismutase (SOD1), is found mainly in the cytosol but also in the mitochondrial intermembrane space, lysosomes (organelles containing hydrolytic enzymes), and the nucleus.^{105,106} Manganese superoxide dismutase (SOD2) acts in the mitochondria.^{106,107} The superoxide dismutases catalyze conversion of O₂.⁻ to H₂O₂, which is in turn converted to water and oxygen by catalase and glutathione peroxidase. Catalase (CAT) is found mainly in peroxisomes, organelles that sequester multiple oxidative enzymes for metabolism of endocytosed molecules such as fatty acids.¹⁰⁸ These peroxisomal enzymes produce H₂O₂ as a byproduct of their reactions, so it is important that CAT is present to neutralize it.¹⁰⁸ Glutathione peroxidase (GPX) is mainly cytosolic but has also been identified in the mitochondrial matrix (about 10% of its distribution) and in the nucleus. It catalyzes the neutralization of H₂O₂ to H₂O outside peroxisomes by a coupled oxidation reaction of reduced glutathione (GSH) to form a dimer (GSSH), which is then recycled by the enzyme glutathione reductase.¹⁰⁹ Other enzymes such as glucose-6-phosphodiesterase (G6PD), glutathione-S-transferase, glutathione reductase,¹¹⁰ an extracellular form of SOD,¹¹¹ and heme oxygenase¹¹² also play significant roles in antioxidant protection.

FIG. 4 also depicts an important phenomenon, the Fenton reaction, which is a kind of Haber-Weiss reaction specifically involving iron. In Haber-Weiss reactions, H₂O₂ is converted to the highly damaging OH. in the presence of cationic metals such as ferrous and cupric ions.^{77,113} Because O₂.⁻ promotes the Fenton reaction by mobilizing and regenerating iron from ferritin and iron-sulphur clusters in enzymes, SOD enzymes serve an important role by shifting the equation away from O₂.⁻-mediated OH. production and toward the formation of H₂O₂.¹¹³ O₂.⁻ also reacts with NO. to form ONOO−, which quickly protonates to form another highly reactive species.^{4,114} Similarly, superoxide's dismutation product, H₂O₂, can degrade heme proteins, liberating bound iron and promoting the Fenton reaction as well as providing the substrate for generation.¹¹⁵ Thus, a balance of antioxidants is required to control both key ROS and their products.¹¹⁶

### Antioxidant Defense and Aging

There is no conclusive evidence to date that antioxidant enzyme defense fails with age in normal human dermal fibroblasts, or that there are significant postnatal age-associated changes in mRNA, protein levels, or enzyme activity.¹¹⁷ Allen et al. found that SOD1 and SOD2 display increased enzyme activities, protein levels and mRNA abundance in postnatal human fibroblasts when compared to fetal fibroblasts (12-20 weeks gestation), but there was no significant difference in these parameters among postnatal age groups (17-33 years old versus 78-94 years old).¹¹⁸ The same group found that GPX enzyme activity and mRNA abundance in human fibroblasts were also increased postnatally compared to fetal fibroblasts, but no changes in GPX activity among postnatal ages were detected, though there was a decrease in total glutathione protein (the substrate for GPX),¹¹⁹ In contrast to these findings, there are reports of decreased expression and response to signaling in antioxidant enzymes such as SOD2 in other cell types such as skeletal muscle,^{10,120} and a decrease in other antioxidant enzyme function such as glutamine synthetase (GS) and glucose-6-P dehydrogenase (G-6-PDH) activities in aged rat liver and brain tissue.¹²¹ It is possible that, due to the genetic variations in AOE expression and activity levels between individuals, the best way to determine age-related changes in AOE is to follow an individual through life.

Plasma redox balance is reported to shift significantly toward oxidation between the 3^{rd} and 10^{th} decades of life, although the exact reason is unknown.⁴ This may in part explain why markers of net oxidative damage increase with age, such as oxidative DNA damage (measured by 8-oxo-dG),⁸¹ protein carbonyls,^{122,123} lipid peroxidates,¹²³ and enzymes with decreased function and stability.¹²² Irreversible glycation products of proteins and the amino groups on lipids and DNA, called advanced glycation end-products (AGE), accumulate with age.¹²⁴ AGE are implicated in major diseases such as diabetes, atherosclerosis and Alzheimer's.^{125,126} The reason for AGE increase with age is not fully understood; it could involve a net increase in ROS production, decreased efficiency of AGE repair, steady accumulation throughout life, or any combination of these. It has been proposed that at least one reason for AGE increase is upregulation of the immunoglobulin type receptor for AGE (RAGE), which binds AGE in a very stable manner and leads to pathologic cell signaling.¹²⁴

Another important observation associated with aging is a decreased response to cell signaling. It has been shown that in cardiac myocytes, which are a good model for adaptive responses in the context of exercise-induced conditioning, aging alters responses to signaling proteins such as heat shock protein 70, nitric oxide synthase, and oxidative stress-responsive mitogen-activated protein kinases JNK, ERK and p38.^{121,128} In the skin, aged dermal fibroblasts display significantly decreased proliferation in response to epidermal growth factor (EFG) due to decreases in the number of EGF receptors, receptor affinity for ligand, and internalization of ligand-receptor complexes.^{129,130}

### Oxidative Stress and Lifespan

Many proven means of extending lifespan in a species involve modulation of oxidative metabolism or oxidative stress. Longevity has been correlated with efficiency of DNA repair enzymes and SOD enzyme activities per unit metabolic rate.¹³¹ SOD2(−/−) mice die within 10 days of birth, displaying dilated cardiomyopathy and metabolic abnormalities that result in acidosis and lipid accumulation in skeletal muscle and liver.^{132,133} It was recently shown that treating the nematode Caenorhabditis elegans with SOD/CAT mimetics increased their mean and maximum lifespan.¹³⁴

Caloric restriction increases lifespan in mammals, and this has generally been attributed to a reduction of metabolic burden, with reduced generation of O₂.⁻ and H₂O₂ in the mitochondria.^{82,84,135} However, recent work by Lin et al. at MIT revealed that calorie restriction actually increases oxidative metabolism.¹³⁶ They propose that histone deacetylase Sir2 in yeast and the mammalian homolog Sirt1 are key regulators in calorie restriction-related longevity, via mechanisms that are still under investigation but may include modulation of mitochondrial electron transport efficiency, decreased ROS production, increased cellular sensitivity to insulin signaling, and resistance to apoptosis.¹³⁷ There is early evidence that Sirt1 may promote increased resistance to oxidative stress and heat stress by histone deacetylation-mediated repression of proapoptotic stress-response transcription factors including p53, p66shc, forkhead (FOXO) and Bax,^{137,138} as well as the induction of DNA repair gene GADD45¹³⁷ and SOD2.¹³⁹

Inactivation of p66shc, a transcription factor modulator and Ras/MAPK signaling protein, has been correlated with increased lifespan by other groups.¹⁴⁰ It was recently reported to be regulated by p53 in redox responses and apoptosis as well as directly modified by oxidative stress and UV.^{140,141} This was shown using p66shc knock-out mice; in addition to a 30% increased lifespan, the murine cells were found to have reduced intracellular ROS levels and decreased oxidative DNA damage.¹⁴¹

In summary, lifespan extension appears to involve a combination of prompt stress responses, resistance against cumulative oxidative damage, and metabolic efficiency.

### SUMMARY OF THE INVENTION

The present invention is related to the use of a telomere homolog oligonucleotide (t-oligo or TO) for treating a subject in need of a treatment for an oxidative stress disorder. The t-oligo may be an oligonucleotide with at least 33% sequence identity with (TTAGGG)ₙ, wherein n can be any number from 1 to 333. The sequence identity may be at least 50%. The oligonucleotide may be pGAGTATGAG (SEQ ID NO: 2), pGTTAGGGTTAG (SEQ ID NO: 1), pGGGTTAGGGTT (SEQ ID NO: 3), pTAGATGTGGTG (SEQ ID NO: 4) and pTT. The oligonucleotide may be GAGTATGAG (SEQ ID NO: 5), GTTAGGGTTAG (SEQ ID NO: 6), GGGTTAGGGTT (SEQ ID NO: 7), TAGATGTGGTG (SEQ ID NO: 8) and TT. The subject may be a human.

The oxidative stress disorder may be retinal degeneration, Alzheimer's disease, aging, photoaging, skin photoaging and cardiovascular disease. The cardiovascular disease may be hypertension, hypercholesterolemia, diabetes mellitus, and hyperhomocysteinemia. The subject may be undergoing a treatment that causes the oxidative stress disorder. The treatment may be a cancer treatment, such as chemotherapy or radiation therapy.

The present invention is also related to a method of screening for modulators of oxidative stress. The method comprises contacting a cell (preferably under oxidative stress) with a candidate modulator. The level of telomere disruption is then measured in the cell. A modulator is identified by altering the level of telomere disruption compared to a control, comprising a cell not subjected to oxidative stress and a cell subjected to oxidative stress, but not exposed to a candidate modulator.

The present invention also relates to methods of treating a subject for an oxidative stress disorder with a composition comprising one or more oligonucleotides, said oligonucleotide having between 2 and 200 bases and having at least 33% but less than 100% identity with the sequence (TTAGGG)ₙ, and optionally having a 5′ phosphate, and when said oligonucleotide comprises the sequence 5′-RRRGGG-3′ (R=any nucleotide) said oligonucleotide has a guanine content of 50% or less. The oligonucleotide may lack cytosine.

The present invention also relates to methods of preventing an oxidative stress disorder with a composition comprising one or more oligonucleotides, said oligonucleotide having between 2 and 200 bases and having at least 33% but less than 100% identity with the sequence (TTAGGG)ₙ, and optionally having a 5′ phosphate, and when said oligonucleotide comprises the sequence 5′-RRRGGG-3′ (R=any nucleotide) said oligonucleotide has a guanine content of 50% or less. The oligonucleotide may lack cytosine.

The methods of the instant invention also include methods of treatment and prevention of oxidative stress with a composition in which an oligonucleotide comprises one or more sequences selected from the group consisting of TT, TA, TG, AG, GG, AT, GT, TTA, TAG, TAT, ATG, AGT, AGG, GAG, GGG, TTAG, TAGG, AGGG, GGTT, GTTA, TTAGG, TAGGG, GGTTA, GTTAG, GGGTT and GGGGTT.

The methods of the instant invention also include methods of treatment and prevention of oxidative stress with a composition in which an oligonucleotide is between 40% and 90% identical to (TTAGGG)ₙ.

The methods of the instant invention also include methods of treatment and prevention of oxidative stress with a composition in which an oligonucleotide is selected from the group consisting of oligonucleotides 2-200 nucleotides long; oligonucleotides 2-20 nucleotides long; oligonucleotides 5-16 nucleotides long; and oligonucleotides 2-5 nucleotides long.

The methods of the instant invention also include methods of treatment and prevention of oxidative stress with a composition in which an oligonucleotide is selected from the group consisting of: GTTAGGGTGTAGGTTT (SEQ ID NO: 9); GGTTGGTTGGTTGGTT (SEQ ID NO: 10); GGTGGTGGTGGTGGT (SEQ ID NO: 11); GGAGGAGGAGGAGGA (SEQ ID NO: 12); GGTGTGGTGTGGTGT (SEQ ID NO: 13); TAGTGTTAGGTGTAG (SEQ ID NO: 14); GAGTATGAG (SEQ ID NO: 5); AGTATGA; GTTAGGGTTAG (SEQ ID NO: 6); GGTAGGTGTAGGATT (SEQ ID NO: 15); GGTAGGTGTAGGTTA (SEQ ID NO: 16); GGTTAGGTGTAGGTT (SEQ ID NO: 17); GGTTAGGTGGAGGTTT (SEQ ID NO: 18); GGTTAGGTTAGGTTA (SEQ ID NO: 19); GTTAGGTTTAAGGTT (SEQ ID NO: 20); and GTTAGGGTTAGGGTT (SEQ ID NO: 21).

The invention also relates to methods and compositions for preventing and treating photoaging. Such compositions may comprise a telomere homolog oligonucleotide which may be selected from any of the following oligonucleotides or a combination thereof: an oligonucleotide that has at least 33% sequence identity to (TTAGGG)ₙ, wherein n is a number from 1 to 333; an oligonucleotide has at least 50% sequence identity to (TTAGGG)ₙ, wherein n is a number from 1 to 333; an oligonucleotide that is selected from the group consisting of GAGTATGAG (SEQ ID NO: 5), GTTAGGGTTAG (SEQ ID NO: 6), GGGTTAGGGTT (SEQ ID NO: 7), TAGATGTGGTG (SEQ ID NO: 8) and TT, said oligonucleotide optionally comprising a 5′-phosphate; an oligonucleotide having between 2 and 200 bases and having at least 33% but less than 100% identity with the sequence (TTAGGG)ₙ, and optionally having a 5′-phosphate, and when said oligonucleotide comprises the sequence 5′-RRRGGG-3′ (R=any nucleotide) said oligonucleotide has a guanine content of 50% or less; an oligonucleotide that completely lacks cytosine; an oligonucleotide comprising one or more sequences selected from the group consisting of TT, TA, TG, AG, GG, AT, GT, TTA, TAG, TAT, ATG, AGT, AGG, GAG, GGG, TTAG, TAGG, AGGG, GGTT, GTTA, TTAGG, TAGGG,GGTTA, GTTAG, GGGTT and GGGGTT; an oligonucleotide that is between 40% and 90% identical to (TTAGGG)ₙ, an oligonucleotide that is selected from the group consisting of oligonucleotides 2-200 nucleotides long; oligonucleotides 2-20 nucleotides long; oligonucleotides 5-16 nucleotides long; and oligonucleotides 2-5 nucleotides long; and finally, an oligonucleotide that is selected from the group consisting of GTTAGGGTGTAGGTTT (SEQ ID NO: 9); GGTTGGTTGGTTGGTT (SEQ ID NO: 10); GGTGGTGGTGGTGGT (SEQ ID NO: 11); GGAGGAGGAGGAGGA (SEQ ID NO: 12); GGTGTGGTGTGGTGT (SEQ ID NO: 13); TAGTGTTAGGTGTAG (SEQ ID NO: 14); GAGTATGAG (SEQ ID NO: 5); AGTATGA; GTTAGGGTTAG (SEQ ID NO: 6); GGTAGGTGTAGGATT (SEQ ID NO: 15); GGTAGGTGTAGGTTA (SEQ ID NO: 16); GGTTAGGTGTAGGTT (SEQ ID NO: 17); GGTTAGGTGGAGGTTT (SEQ ID NO: 18); GGTTAGGTTAGGTTA (SEQ ID NO: 19); GTTAGGTTTAAGGTT (SEQ ID NO: 20); and GTTAGGGTTAGGGTT (SEQ ID NO: 21).

The cosmetic composition may comprise a lotion or any other dermatologically acceptable carriers.

### BRIEF DESCRIPTION OF THE DRAWINGS

FIG. 1 shows the telomere loop structure and 3′ overhang sequence. Chromosomes end with telomeres, which contain single-stranded DNA that is looped and secured by several proteins, including TRF1, TRF2 and Pot1, into the proximal double-stranded telomere region (at the d loop) to form a physical cap called a t loop. The single-stranded 3′ overhang sequence in human telomeres consists of tandem repeats of TTAGGG.

FIG. 2 shows the responses to telomere loop disruption. Considerable evidence supports that telomere loop disruption is the key event triggering multiple DNA damage responses. Shown is a summary of ways to disrupt the t loop or mimic t loop exposure and the resulting signaling and adaptive responses published to date. Rectangles highlight findings using mimicked t loop disruption using (TTAGGG)ₙ oligonucleotides. Ovals highlight findings using pTT or TO, which overlap with the other findings.

FIG. 3 shows a model of DNA damage response to oxidative telomere loop disruption in fibroblasts. Telomeres are rich in guanine bases, which are known to be susceptible to oxidative modification. Oxidative stress is known to cause accelerated telomere shortening and cell senescence, in part by decreased binding of telomere binding proteins TRF1 and TRF2. This figure shows the structure of the major oxidative guanine modification, 8-oxo-dG, and its proposed disruption of the telomere loop structure, leading to DNA damage signaling and adaptive responses.

FIG. 4 shows the major antioxidant enzymes (AOE) and reactive oxygen species. Many more enzymes and reactive oxygen and nitrogen species are known to participate in biological reactions. Only major chemical species are shown, without stoichiometry. Abbreviations of enzymes are as follows: SOD1-cytoplasmic superoxide dismutase SOD2-mitochondrial superoxide dismutase CAT-catalase, mostly localized to peroxisomes GPX-glutathione peroxidase, mainly found in cytoplasm and nucleus GSH-reduced glutathione protein, a major antioxidant protein GSSH-oxidized dimer of GSH

FIG. 5 shows that superoxide dismutase mRNAs are not modulated by pTT. (Panel A): These are representative examples of SOD1 and SOD2 mRNA levels during pTT treatment in the same donor fibroblasts. (Panel B): Each graph represents data from three different donors (means±SEM). Values were corrected for loading based on ribosomal 18S RNA bands using densitometry.

FIG. 6 shows that catalase and glutathione peroxidase mRNAs are not modulated by pTT. Shown here are blots showing no modulation of CAT (Panel A) or GPX (Panel B) mRNA levels in the presence of pTT as compared to those in diluents-treated fibroblasts. Results are representative of data from 2-3 donors.

FIG. 7 shows that mitochondrial superoxide dismutase protein is upregulated during pTT treatment. (Panel A): This is a representative Western blot showing AOE protein levels during pTT treatment in the same donor cells. Only SOD2 is consistently modulated, displaying elevated levels through 48 hours compared to diluent-treated cells, in which SOD2 gradually decrease with time. (Panel B): This figure represents the mean induction of SOD2 protein from three donors (mean±SEM). Values were corrected for loading based on Coomassie blue staining using densitometry.

FIG. 8 shows that pTT treatment slows cell growth but does not decrease cell viability. (Panel A): 100 μM pTT treatment significantly decreases fibroblast cell yields as measured by Coulter counts (2-way ANOVA, p=0.0079). The data combine four experiments (mean±SEM). (Panel B): However, viability is not significantly decreased, as measured by the MTS assay (2-way ANOVA, p=0.2588), conducted in parallel with the same donor cells. The data combine four experiments (mean±SEM).

FIG. 9 shows that cell yields are increased after pTT pretreatment. Cell yields are higher in fibroblasts pretreated with 100 μM pTT for 72 hours, replated and grown in regular culture medium, as compared to cells pretreated with diluent and replated at the same density (2-way ANOVA, p=0.0006). The data combine four experiments (mean±SEM).

FIG. 10 shows that pTT pretreatment results in higher cell yields following hydrogen peroxide challenge as compared to diluent pretreatment. (Panel A): Fibroblast cell yields after 25 μM H₂O₂ treatment show higher yields in pTT-pretreated cells (2-way ANOVA, p=0.0008). (Panel B): Cell yields expressed as a percentage of respective controls untreated with H₂O₂ (shown in FIG. 8) were significantly higher in pTT-treated cells (General Linear Model, p=0.05). The data combine four experiments (mean±SEM).

FIG. 11 shows that t-oligos stimulate intracellular ROS production in a sequence specific manner. Dichlorofluorescein diacetate (DCF) fluorescence increases in the presence of increased intracellular ROS. These representative FACScan analysis plots show that both pTT and TO stimulate increases in ROS-dependent fluorescence as compared to control oligonucleotides and diluent. pTT was compared to diluent and pCC controls. TO was compared to diluent and pCTAACCCTAAC (TOC1, SEQ ID NO: 22) and the unrelated sequence pGATCGATCGAT (TOC2, overlapping with diluent curve, SEQ ID NO: 23). All p values for one-way ANOVA comparing groups were ±0.01.

FIG. 12 shows that t-oligo stimulation of ROS is p53-dependent. DCF fluorescence is increased by T-oligo treatment in fibroblasts with wild-type p53, but not in fibroblasts transfected with a dominant negative p53. The above FACS fluorescence plots are examples representative of two experiments performed in duplicate. Diluent, pCC, TOC1 and TOC2 were used as controls.

FIG. 13 shows that NAD(P)H oxidase inhibition abrogates T-oligo-induced ROS production. Treatment of fibroblasts with the NAD(P)H oxidase inhibitor diphenyliodonium chloride (DPI) abrogates the increase in ROS caused by pTT or TO treatment as measured by DCF fluorescence. This provides evidence that the source of the increased ROS is an NAD(P)H oxidase. Results shown were consistently reproducible for pTT and TO six times.

FIG. 14 shows the time course of ROS stimulation: T-oligos versus control oligonucleotides. (Panel A): This figure shows timepoints at 1, 4, 8, 12, 24, 36 and 48 hours, with pTT- and TO-stimulated measurable ROS starting at 36 hours for these donor cells. (Panel B): Oligonucleotide controls showed ROS levels equivalent to diluent treatment levels at all timepoints examined. A representative result at 48 hours is shown here. All data are representative of 3 different time course experiments (for Panels A and B) except for variations in the time ROS induction is first measured (see combined data in FIG. 16).

FIG. 15 shows that pTT stimulates ROS earlier while the 11 mer T-oligo stimulates ROS later but with higher amounts compared to diluent treatment. DCF fluorescence measured ROS levels were increased as early as after 16 hours of treatment with 100 μM pTT. Average induction of ROS was later with 40 μM TO but levels ultimately were higher than those stimulated by pTT. Data reflect three experiments (mean±SEM).

FIG. 16 shows a time course of ROS stimulation, p53 induction/activation and p21 levels in response to T-oligos. Shown is a representative time course experiment measuring stimulation of ROS (Panel A), total p53 protein (measured by antibody DO-1), activated p53 (measured by serine-15 phosphorylation) and p21/Cip1/Waf1 protein levels by a Western blot (Panel B) conducted in parallel with the DCF experiment. Shown is one of two reproducible experiments of two that confirm multiple previous publications measuring p53 and p21 modulations by T-oligos. Due to donor variability, here TO stimulates measurable ROS by 36 hours while pTT shows a small increase at 16 hours.

FIG. 17 shows a dose response study of pTT vs pGTTAGGGTTAG (SEQ ID NO: 1). (Panel A): Assessment of propidium iodide (PI) staining as a measure of toxicity, comparing diluent treatment with 1 mM H₂O₂ immediately after DCF incubation. (Panel B): PI fluorescence in pTT and TO samples with increasing doses are comparable to diluent treatment. (Panel C): Representative DCF fluorescence peaks, showing highest fluorescence in each category measured. DCF is not saturated by TO since 1 mM H2O2 stimulates greater DCF fluorescence. (Panel D) There is a significant difference in ROS stimulation between doses and treatment group for 25 μM, 40 μM and 100 μM doses (2-way ANOVA, p=0.0038).

FIG. 18 shows that senescence is not a major response to limited T-oligo treatment. Only 40 μM TO displayed a modest but significant increase in cells staining positively for the SA-â-gal assay for senescence within 24-72 hours, as compared to 100 μM pTT and diluent control (2-way ANOVA comparing treatment groups over time, p<0.01, with post hoc analysis identifying only TO as significantly different from diluent and pTT, which are not statistically different). The same donor cell mixtures were used as in the DCF time course experiments. Data combine three experiments (mean±SEM).

FIG. 19 shows that pTT does not stimulate release of extracellular hydrogen peroxide. Cells were treated for two days with 100 μM pTT, pAA or diluent as control before being assayed for extracellular H₂O₂ production by the horseradish peroxidase assay. Data is a representative of four experiments using triplicate plates, showing no increase in any treatment group over control samples (1-way ANOVA p>0.05, with post hoc comparison of each group not significantly different from HRP(−) negative controls).

FIG. 20 shows that T-oligo pGTTAGGGTTAG (TO, SEQ ID NO: 1) treatment increases resistance of treated cells to H₂O₂-induced stress. A. Cell yields determined up to 48 hours after oxidative challenge display increased resistance to H₂O₂ in T-oligo-treated cultures as determined by increased cell yield. B. Cell yields in H₂O₂ stimulated TO-pretreated and control cultures were calculated as percent of their own diluent control.

FIG. 21 shows Western blot analysis of TO-oligo treated newborn fibroblasts with SOD1, SOD2, Catalase, Glutathione Peroxide and actin specific antibodies.

FIG. 22 shows that reactive oxygen species, telomeres and T-oligos. This figure summarizes the findings in this investigation: effects on cell growth, SOD2 protein, and p53- and NADPH oxidase-dependent ROS production. This supports the hypothesis that T-oligos stimulate DNA damage and adaptive responses in part by modulating the production of ROS. Signaling relationships based on literature are drawn in gray while steps in the hypothesis and from current experimental findings are drawn in black. Question marks highlight relationships that are described in the literature but require further studies for confirmation.

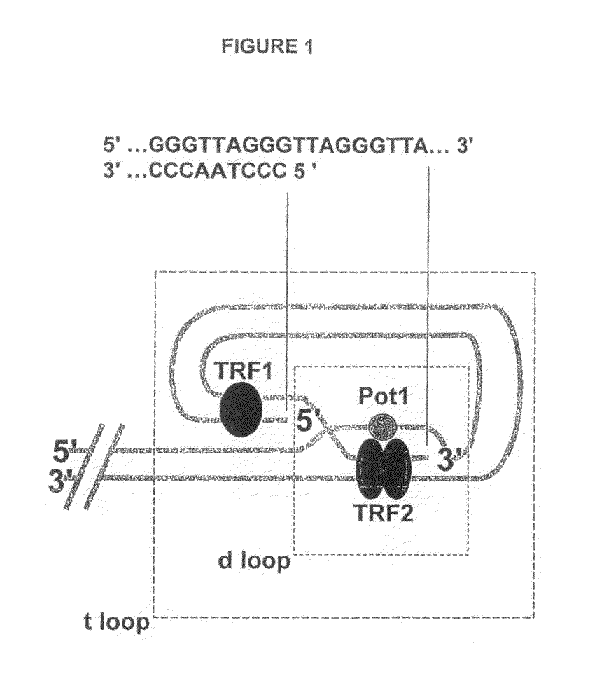

### DETAILED DESCRIPTION

### Treatment for Oxidative Stress

The present invention is related to the discovery that t-oligos affect the redox state of mammalian cells through p53-dependent induction of ROS from NAD (P)H oxidases, which leads to enhanced resistance to future genotoxic stress such as oxidative stress and oxidative damage, including, but not limited to, photoaging. As a result of these novel properties, t-oligos may be used for treating a subject in need of treatment of an oxidative stress disorder. The subject may be any mammal, such as a human. Representative examples of oxidative stress disorders include, but are not limited to, retinal degeneration, Alzheimer's disease, aging, photoaging, skin photoaging and cardiovascular disease, such as hypertension, hypercholesterolemia, diabetes mellitus, and hyperhomocysteinemia.

All oligonucleotides disclosed in this specification are oriented 5′ to 3′, left to right in agreement with standard usage.

The oxidative stress disorder may also be caused by a treatment for another disorder. The t-oligo may be used in such cases to minimize oxidative stress side effects caused by another treatment. For example, many cancer therapies, such as chemotherapy and radiation therapy can cause oxidative stress in the patient, which leads to many of the side effects associated with cancer therapies. T-oligos may be used to reduce the side effects of such cancer treatments.

As used herein, the term “treat” or “treating” when referring to protection of a subject from a condition, means preventing, suppressing, repressing, or eliminating the condition. Preventing the condition involves administering a composition of the present invention to a subject prior to onset of the condition. Suppressing the condition involves administering a composition of the present invention to a subject after induction of the condition but before its clinical appearance. Repressing the condition involves administering a composition of the present invention to a subject after clinical appearance of the condition such that the condition is reduced or prevented from worsening. Elimination the condition involves administering a composition of the present invention to a subject after clinical appearance of the condition such that the mammal no longer suffers the condition.

### T-Oligo

The t-oligo may be a telomere homolog oligonucleotide that induces in cells the same DNA damage responses as telomere-loop disruption. T-oligos are further described in U.S. Pat. Nos. 5,643,556, 5,955,059, 6,147,056 and U.S. patent application Ser. Nos. 10/122,630 and 10/122,633, 11/195,088, the contents of which are incorporated by reference. The t-oligo may have at least 50% nucleotide sequence identity to the telomere repeat sequence of the subject. In vertebrates, the telomere overhang repeat sequence is (TTAGGG)ₙ, where n is from about 1 to about 333. The t-oligo may also have at least 33%, 50%, 60%, 70%, 80%, 90%, 95% or 100% nucleotide sequence identity to the telomere repeat sequence. Representative examples of t-oligos include, but are not limited to, pGAGTATGAG (SEQ ID NO: 2), pGTTAGGGTTAG (SEQ ID NO: 1), pGGGTTAGGGTT (SEQ ID NO: 3), pTAGATGTGGTG (SEQ ID NO: 4), pTAGGAGGAT (SEQ ID NO: 24), pAGTATGA, pGTATG, pTT, GAGTATGAG (SEQ ID NO: 5), GTTAGGGTTAG (SEQ ID NO: 6), GGGTTAGGGTT (SEQ ID NO: 7), TAGATGTGGTG (SEQ ID NO: 8), TAGGAGGAT (SEQ ID NO: 25), AGTATGA, GTATG and TT.

The t-oligo may be of a form including, but not limited to, single-stranded, double-stranded, or a combination thereof. The t-oligo may be phosphorylated at its 5′-end. The t-oligo may comprise a single-stranded 3′-end of from about 2 to about 2000 nucleotides, more preferably from about 2 to about 200 nucleotides. Also specifically contemplated is an analog, derivative, fragment, homolog or variant of the t-oligo.

The t-oligo may used in a composition of one or more oligonucleotides that have between 2 and 200 bases and that are at least 33% but less than 100% identical with the sequence (TTAGGG)ₙ, and that optionally have a 5′ phosphate. T-oligo may be an oligonucleotide that comprises the sequence 5′-RRRGGG-3′, wherein R equals any nucleotide and wherein the oligonucleotide has a guanine content of 50% or less. The T-oligo may lack cytosine.

T-oligo may comprise one or more sequences selected from the group consisting of TT, TA, TG, AG, GG, AT, GT, TTA, TAG, TAT, ATG, AGT, AGG, GAG, GGG, TTAG, TAGG, AGGG, GGTT, GTTA, TTAGG, TAGGG, GGTTA, GTTAG, GGGTT and GGGGTT.

T-oligo may be selected from the group consisting of oligonucleotides 2-200 nucleotides long; oligonucleotides 2-20 nucleotides long; oligonucleotides 5-16 nucleotides long; and oligonucleotides 2-5 nucleotides long.

T-oligo may be selected from the group consisting of GTTAGGGTGTAGGTTT (SEQ ID NO: 9); GGTTGGTTGGTTGGTT (SEQ ID NO: 10); GGTGGTGGTGGTGGT (SEQ ID NO: 11); GGAGGAGGAGGAGGA (SEQ ID NO: 12); GGTGTGGTGTGGTGT (SEQ ID NO: 13); TAGTGTTAGGTGTAG (SEQ ID NO: 14); GAGTATGAG (SEQ ID NO: 5); AGTATGA; GTTAGGGTTAG (SEQ ID NO: 6); GGTAGGTGTAGGATT (SEQ ID NO: 15); GGTAGGTGTAGGTTA (SEQ ID NO: 16); GGTTAGGTGTAGGTT (SEQ ID NO: 17); GGTTAGGTGGAGGTTT (SEQ ID NO: 18); GGTTAGGTTAGGTTA (SEQ ID NO: 19); GTTAGGTTTAAGGTT (SEQ ID NO: 20); and GTTAGGGTTAGGGTT (SEQ ID NO: 21).

### Composition

The present invention also relates to a composition comprising a t-oligo. The composition may also comprise an additional therapeutic, such as an antioxidant. The composition may be a cosmetic composition and may additionally comprise a dye, fragrance and any other component commonly used in a cosmetic industry.

The compositions may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

The compositions may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

The compositions may also be formulated as suppositories, which may contain suppository bases including, but not limited to, cocoa butter or glycerides. Compositions of the present invention may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. Compositions of the present invention may also be formulated transdermal formulations comprising aqueous or nonaqueous vehicles including, but not limited to, creams, ointments, lotions, pastes, medicated plaster, patch, or membrane.

The compositions may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The compositions may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

The compositions may also be formulated as a liposome preparation. The liposome preparation can comprise liposomes which penetrate the cells of interest or the stratum corneum, and fuse with the cell membrane, resulting in delivery of the contents of the liposome into the cell. For example, liposomes such as those described in U.S. Pat. No. 5,077,211 of Yarosh, U.S. Pat. No. 4,621,023 of Redziniak et al. or U.S. Pat. No. 4,508,703 of Redziniak et al. can be used. The compositions of the invention intended to target skin conditions can be administered before, during, or after exposure of the skin of the mammal to UV or agents causing oxidative damage. Other suitable formulations can employ niosomes. Niosomes are lipid vesicles similar to liposomes, with membranes consisting largely of non-ionic lipids, some forms of which are effective for transporting compounds across the stratum corneum.

The compositions may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular.

A therapeutically effective amount of the composition required for use in therapy varies with the nature of the condition being treated, the length of time that activity is desired, and the age and the condition of the subject, and is ultimately determined by the attendant physician. In general, however, doses employed for adult human treatment typically are in the range of 0.001 mg/kg to about 200 mg/kg per day. The dose may be about 1 μg/kg to about 100 μg/kg per day. The desired dose may be conveniently administered in a single dose, or as multiple doses administered at appropriate intervals, for example as two, three, four or more subdoses per day. Multiple doses often are desired, or required.

The dosage of a composition may be at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg or 1 mg/kg (active ingredient per weight of subject)

### Screening Methods

The present invention also relates to screening methods of identifying modulators of oxidative stress. The screening methods may be performed in a variety of formats including, but not limited to, in vitro, cell-based, genetic and in vivo assays. A modulator may be identified by screening for substances that affect the structure of telemores, which may be determined by measuring modulation of apoptosis, senescence, or the activity or phosphorylation of p53 or p95. Modulation of apoptosis may be measured by methods including, but not limited to, measuring the size of the sub-G₀/G₁ peak in FACS analysis, TUNEL assay, DNA ladder assay, annexin assay, or ELISA assay. Modulation of senescence may be determined by measuring senescence-associated β-galactosidase activity or failure to increase cell yields or to phosphorylate pRb or to incorporate ³H-thymidine after mitogenic stimulation. Modulation of p53 activity may be determined by measuring phosphorylation of p53 at serine 15 by gel shift assay by p53 promoter driven CAT or luciferase construct read-out, or by induction of a p53-regulated gene product such as p21. Modulation of p95 activity may be determined by measuring phosphorylation of p95 at serine 343 by shift in the p95 band in a western blot analysis, or by FACS analysis to detect an S phase arrest.

Any cells may be used with cell-based assays, such as mammalian cells including human and non-human primate cells. Representative examples of suitable cells include, but are not limited to, primary (normal) human dermal fibroblasts, epidermal keratinocytes, melanocytes, and corresponding immortalized or transformed cell lines; and primary, immortalized or transformed murine cells lines. The amount of protein phosphorylation may be measured using techniques standard in the art including, but not limited to, colorimetery, luminometery, fluorimetery, and western blotting.

Conditions, under which a suspected modulator is added to a cell, such as by mixing, are conditions in which the cell can undergo apoptosis or signaling if essentially no other regulatory compounds are present that would interfere with apoptosis or signaling. Effective conditions include, but are not limited to, appropriate medium, temperature, pH and oxygen conditions that permit cell growth. An appropriate medium is typically a solid or liquid medium comprising growth factors and assimilable carbon, nitrogen and phosphate sources, as well as appropriate salts, minerals, metals and other nutrients, such as vitamins, and includes an effective medium in which the cell can be cultured such that the cell can exhibit apoptosis or signaling. For example, for a mammalian cell, the media may comprise Dulbecco's modified Eagle's medium containing 10% fetal calf serum.

Cells may be cultured in a variety of containers including, but not limited to tissue culture flasks, test tubes, microtiter dishes, and petri plates. Culturing is carried out at a temperature, pH and carbon dioxide content appropriate for the cell. Such culturing conditions are also within the skill in the art.

Methods for adding a suspected modulator to the cell include electroporation, microinjection, cellular expression (i.e., using an expression system including naked nucleic acid molecules, recombinant virus, retrovirus expression vectors and adenovirus expression), adding the agent to the medium, use of ion pairing agents and use of detergents for cell permeabilization.

Candidate modulators may be naturally-occurring molecules, such as carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such peptidomimetics and the like; and non-naturally occurring molecules, such as “small molecule” organic compounds. The term “small molecule organic compound” refers to organic compounds generally having a molecular weight less than about 1000, preferably less than about 500.

Candidate modulators may be present within a library (i.e., a collection of compounds), which may be prepared or obtained by any means including, but not limited to, combinatorial chemistry techniques, fermentation methods, plant and cellular extraction procedures and the like. Methods for making combinatorial libraries are well-known in the art. See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein.

The present invention has multiple aspects, illustrated by the following non-limiting examples.

### Example 1

### Antioxidant Defense Responses to Telomere Homolog Oligonucleotides

### Adaptive Defense Against Oxidative DNA Damage

Repair of oxidized molecules such as DNA is well-described and necessary for survival and the propagation of species.^{11,90,147} Upregulation of repair mechanisms following DNA damage occurs in both prokaryotes and eukaryotes. In prokaryotes this is called the “SOS response” and requires sensing of single-stranded DNA by a protein that then causes derepression of transcription for multiple adaptive DNA damage responses.¹⁴⁸ There is also considerable evidence that eukaryotic cells adapt to DNA damaging agents, initiating protective responses following noxious stimuli to prevent and/or repair future damage and increase the ability of cells to survive subsequent deleterious conditions.^{90,142,143,148} For example, enhanced resistance to low doses of ionizing radiation, termed the “radioadaptive” response, has been described in several cell types.¹⁴⁵ Ionizing radiation (generally considered X-ray and gamma radiation) is known to cause direct DNA modification such as strand breaks, while UVA produces damage through chromophores that produce ROS.^{113,149} Adaptive responses to oxidative stress, which in vivo may be caused by UV, pollution, cigarette smoke and the endogenous production of ROS by mitochondria and numerous enzymes,¹¹³ is also described. Wiese et al. showed adaptive increases in viability against toxic H₂O₂ concentrations in Chinese hamster ovary fibroblast cultures, after “priming” with low doses of H₂O₂.¹⁴³ In human skin, melanogenesis is considered an adaptive DNA damage response following UV exposure, protecting skin cells from subsequent UV irradiation and potential DNA damage.^{150,151}

Interestingly, recent reports on the radioadaptive response to alpha particles also showed that so-called “bystander cells,” such as human lung fibroblasts that were not irradiated, but were treated with conditioned medium from irradiated cells, displayed elevated levels of the oxidative DNA damage repair protein apurinic endonuclease and had increased colony-forming capacity as compared to cells treated with non-conditioned medium following subsequent irradiation of both groups.¹⁵² This supports the existence of paracrine mechanisms in fibroblast adaptive responses as well as responses to increased intracellular ROS. One recent study suggests that this paracrine signaling may be mediated by H₂O₂ released by ROS-producing enzymes, NADPH oxidases.¹⁵³

### Adaptive Induction of Antioxidant Enzymes

The adaptive response to ionizing radiation includes modulation of antioxidant enzymes.^{145,154} AOE modulation varies greatly with cell type and treatment conditions. They respond to numerous stimuli such as cytokines, hyperoxia, hypoxia, H₂O₂, UV and gamma radiation.^{104,155-158} It has been shown that oxidative stress and ionizing radiation stimulate the activity of antioxidant enzymes (AOE) such as superoxide dismutases (SOD), catalase (CAT) and glutathione peroxidase (GPX), especially mitochondrial superoxide dismutase (SOD2).^{82,104,144,145} Poswig et al. found that cultures from several different fibroblast donors repeatedly exposed to UVA (20 J/cm²) display up to a 5-fold induction of SOD2 mRNA levels following three UVA exposures as compared to sham-irradiated controls.¹⁴⁴ Leccia et al. treated human dermal fibroblasts with physiologic doses of solar-simulated UV and found adaptive modulations in cytoplasmic superoxide dismutase (SOD1), SOD2, and GPX but not CAT over several days following irradiation.¹⁰⁴

### p53 in Antioxidant Defense and Oxidative DNA Damage Repair

Adaptation to oxidative stress also involves p53. It has recently been shown that p53 protein can modulate BER, the repair pathway for oxidative DNA damage.¹⁵⁹⁻¹⁶¹ Offer et al. provide evidence that in gamma-irradiated lymphoid cells p53 modulates BER and apoptosis depending on when damage is detected in the cell cycle.¹⁶² Oxidative stress can also indirectly activate p53 via activation of AP-1 transcription factor, which activates redox factor 1/apurinic endonuclease protein (Ref-1/APE), a protein that not only serves as the key rate-limiting enzyme in BER, but also regulates redox-sensitive transcription factors.¹⁴⁹ Ref-1/APE is reported to activate p53, demonstrating a reciprocal regulatory relationship between p53 and DNA repair that allows a cell to initiate repair, apoptosis, senescence or other responses depending on the sum of stress signals.^{161,163-165}

Interestingly, there is also evidence of a reciprocal relationship between p53 and SOD2, presumably to control amounts of H₂O₂ produced by SODs that then lead to apoptosis.¹⁶⁶ Drane et al. recently showed in the human breast cancer cell line MCF-7 that there is a partial p53 binding site on the SOD2 promoter and that in luciferase reporter gene assays p53 can repress the SOD2 gene promoter.¹⁶⁶ Furthermore, SOD2 overexpression reciprocally repressed p53 expression in their system, which shows that SOD2 serves as a signaling molecule as much as it is a O₂.⁻ neutralizer.¹⁶⁶ SOD1 is also reported to be repressed by p53 at the transcriptional level¹⁶⁷ while GPX is induced,¹⁶⁸ suggesting that p53 actively regulates intracellular ROS levels at least in part through AOE regulation. However, it remains unclear how much functional repression occurs in a physiologic setting, since upregulation of SOD1, SOD2, GPX and p53 are observed in skin cells after UV irradiation.¹⁰⁴ Since ROS have been shown to participate in signaling events leading to cell cycle arrest, senescence and apoptosis, p53-dependent AOE modulation provides one way to control levels of intracellular ROS.^{153,169-171}

### DNA Damage Responses Stimulated by Thymidine Dinucleotide Treatment

Adaptive responses to DNA damage have been reported in the absence of stimuli known to cause DNA damage.^{13,172,173} Several years ago, it was postulated by Eller et al. that excision of DNA photoproducts during their repair after UV exposure is a trigger for melanogenesis, a DNA damage response.¹⁵⁰ Cultured S91 melanoma cells and cultured melanocytes as well as in vivo guinea pig skin treated with solutions of 5′-phosphorylated thymidine dinucleotides (pTT) displayed enhanced melanin production.^{150,151}

It has been since shown that 100 μM pTT, both in vitro and in mouse models in vivo, stimulates enhanced nucleotide excision DNA repair and resistance to subsequent UV irradiation.^{142,174} Multiple key gene products involved in regulating cell cycle checkpoints and DNA damage repair are upregulated by pTT. These include p53, PCNA, GADD45, XPA, ERCC3, and p21.^{142,172,115} Furthermore, it was demonstrated that pTT applied to mouse skin activates tyrosinase^{173,176} and the cytokine TNF-α, and inhibits contact hypersensitivity, effects observed after UVB irradiation.¹⁷⁷

In summary, it has been shown that priming cells in culture with low doses of UV or hydrogen peroxide stimulates antioxidant defense and resistance to subsequent oxidative stress, an important cause of DNA damage. pTT and TO treatment in vitro were found to stimulate many of the same responses observed after UV irradiation, likely by mimicking telomere loop disruption. Human dermal fibroblasts are subject to oxidative stress and DNA damage produced by UV.^{178,179} Therefore, it is hypothesized that pTT treatment in human dermal fibroblasts stimulates the same responses triggered by UV irradiation or telomere loop exposure, including adaptive antioxidant defense. Modulation of antioxidant enzymes and resistance to oxidative stress following pTT treatment or telomere loop disruption has not previously been described. The goal of Example 1 was to investigate the effect of thymidine dinucleotide (pTT) on the mRNA and protein levels of the antioxidant enzymes Cu—Zn superoxide dismutase (SOD1), Mn superoxide dismutase (SOD2), catalase (CAT), and glutathione peroxidase (GPX) in primary human dermal fibroblasts on their resistance to a subsequent H₂O₂ oxidative challenge.

### Fibroblast Cell Culture

Cell culture followed previously published methods.⁹² Primary human newborn dermal fibroblasts were cultured from neonatal circumcised foreskin specimens. The skin samples were treated overnight in a 0.25% trypsin solution at 4° C. to separate the epidermis from the dermis. The separated dermis was then cut into pieces and plated onto etched plastic tissue culture dishes. Primary culture medium consisted of DMEM supplemented with 10% bovine CS, 50 U/ml penicillin and 50 μg/ml streptomycin sulfate. Cells were maintained in incubators at 37° C. and 6% CO₂ for three weeks, reaching 90-95% confluency before use in experiments. Secondary culture medium consisted of DMEM supplemented with 10% CS.

### Chemicals

Hydrogen peroxide (30% w/w, with 0.5 ppm stannate and 1 pmm phosphorus as preservatives) was obtained from Sigma (USP grade, St. Louis, Mo.). The stock bottle was stored at 4° C. and all dilutions were made in DMEM immediately before use.

### Oligonucleotide Preparation and Cell Treatment

Purified 5′-phosphorylated thymidine dinucleotides (pTT) (Midland Certified Reagents, Inc., Texas) purified by gel filtration and analyzed by mass spectroscopy, were obtained in lyophilized form. 5′-phosphorylation was observed in murine melanoma cells to increase nuclear uptake of the oligonucleotides.¹⁷⁵ The lyophilized pTT was resuspended in sterile dH₂O to generate a 2 mM stock solution. The stock solution was syringe filter-sterilized through a 0.2 μm pore filter and spectrophotometrically analyzed (absorbance at 260 and 280 nm) to determine the concentration, and frozen in aliquots at −20° C. The stock solution was further diluted into working concentrations in cell culture media immediately before use. All treatments involved initial one-time treatment with 100 μM pTT, a dose chosen based on previous experiments measuring colony-forming ability after pTT treatment and time course studies showing adaptive induction of p53 and nucleotide excision repair proteins ERCC3, GADD45, and SDI1, without evidence of toxicity.¹⁴²

Cells were harvested at different time intervals without further medium change or addition of more dinucleotide. Diluent alone was used as a control treatment.

### Determination of Cell Yields

Equal numbers of fibroblasts were seeded into 32 mm culture dishes, and paired dishes were treated with pTT or diluent control as described above. At 24, 48 and 72 hours of treatment, the cells were harvested by trypsinization and counted in an automated cell counter (Coulter Z Series, Beckman Coulter, Inc., Fullerton, Calif.). The experiments were conducted in parallel with the MTS assay, using the same donor cells.

### MTS Viability Assay

The CellTiter 96 Aqueous One Solution Cell Proliferation Assay, a version of the MTT assay, (Promega Corp., Madison, Wis.) is generally used as a eukaryotic cell viability and proliferation assay.¹⁸⁰ It has also been used to measure mitochondrial dysfunction,¹⁸¹ because the assay measures the reduction of a tetrazolium compound [3-(4,5-dimethylthiazol-2-yl)-5-(-3-carboxymethoxyphenyl)-2-(4-sulfophenyl)-2H-tetrazolium] to formazan in viable mitochondria. The MTS assay utilizes a water-soluble form of the tetrazolium reagent in the original MIT assay. The formazan product is measured by absorbance at 492 nm.

Equal numbers of cells were seeded into 96-well tissue culture plates, and paired wells were treated once with pTT or diluent control as described above. At 24, 48 and 72 hours cell viability was assayed using the MTS assay and an ELIZA plate reader (Tecan Spectra II Model F039002, Austria) to measure absorbance at 492 nm. The experiments were conducted in parallel with cell yield Coulter counts, using the same donor cells.

### Hydrogen Peroxide Oxidative Challenge

Early passage neonatal foreskin fibroblasts were passed to 100 mm dishes and pretreated 24 hours after passage with 100 μM pTT or diluent control. Cells were harvested and replated after 3 days at a density of 0.5×10⁴ cells/cm² in 35 mm dishes and treated 24 hours later with 25 μM hydrogen peroxide (Sigma, USP grade, St. Louis, Mo.) for one hour at 37° C. and 6% CO₂. For each experiment fresh H₂O₂ solutions were made in DMEM. The H₂O₂ and control DMEM solutions were made and sterilized through 0.45 μm syringe filters immediately before treatment. After the one-hour treatment with H₂O₂ or diluent control, fresh medium was provided. Cells were harvested at later timepoints by a brief washing in 1× EDTA, followed by trypsinization at 37° C. Cell yields were determined using the Coulter Z cell counter.

### Northern Blot Analysis of Antioxidant Enzymes

Cells were harvested in Trizol (Gibco BRL, Gaithersburg, Md.) and stored at −70° C. RNA was purified by phenol/chloroform separation, precipitated by isopropanol, washed with 70% ethanol, and resuspended in RNAse-free dH₂O. RNA solutions were measured by spectrophotometer readings at 260 nm and 280 nm to determine concentration and purity. Equal amounts of total RNA from each sample (3.5 to 10 μg total RNA) were separated in a 1% agarose/6% formaldehyde gel, stained with ethidium bromide, and then transferred by capillarity to Hybond-N nylon membrane (Amersham Pharmacia Biotech, UK Ltd.). Membranes were sequentially hybridized with SOD1, SOD2, CAT and GPX cDNA probes labelled with [³²P]dCTP using the Rediprime II Randome Prime Labelling System protocol (Amersham Biosciences Corp, Piscataway, N.J.). Labelled probe solution yielded at least 20 million counts by scintillation counter (Wallac 1409 Liquid Scintillation Counter, Perkin Elmer Wallac, Inc., Gaithersburg, Md.). Labeled membranes were exposed to XAR film (Eastman Kodak Co.) at −70° C.

SOD 1, SOD2 and CAT cDNA probes were obtained from American Type Culture Collection (ATCC plasmids catalogue #39786, 59946, 57354, respectively, Manassas, Va.) and plasmids were subjected to the appropriate restriction enzyme digestion followed by gel purification. GPX cDNA was generated by RT-PCR using human fibroblast RNA, followed by sequencing and column purification.¹⁰⁴ The primer sequences used for GPX cDNA generation were 5′-CTACTTATCGAGAATGTGGCG-3′ (SEQ ID NO: 26) and 5′-CGATGTCAATGGTCTGGAAG-3′ (SEQ ID NO: 27).¹⁰⁴

### Western Blot Analysis of Antioxidant Enzymes

Cell lysates were harvested at various intervals after T-oligo treatment in harvest buffer containing 0.25 M Tris HCl (pH 7.5), 0.375 M NaCl, 2.5% sodium deoxycholate, 1% Triton X-100, 25 mM MgCl₂, 0.1 mg/ml aprotinin (Sigma, St. Louis, Mo.) and 1 mM phenylmethyl sulfonyl flouride (PMSF) (Sigma). Samples were sheared through a fine needle syringe, sonicated, centrifuged and the supernatant was isolated. Total cellular protein concentrations were determined spectrophotometrically with the Bio-Rad protein assay (Bio-Rad Laboratories, Inc, Hercules, Calif.). Equal amounts of total protein from each sample (35-65 μg) were separated by 10-15% polyacrylamide gel electrophoresis, and transferred to nitrocellulose membranes. After transfer, gels were stained with Coomassie Blue (Sigma) to ascertain evenness of loading.

Membranes were reacted with antibodies diluted in Tris-based buffer with nonfat milk powder as a blocking agent. Antibodies against SOD1 (1:250 dilution, BD Biosciences, San Diego, Calif.), SOD2 (1:200 dilution, The Binding Site, San Diego, Calif.), CAT (1:1000 dilution, Calbiochem, San Diego, Calif.), GPX (1:1000 dilution, Biodesign International, Saco, Minn.) were reacted to membranes, followed by appropriate secondary antibodies diluted at 1:2000 (Biorad Laboratories, Inc., Hercules, Calif.). Antibody binding was detected with electrochemical luminescence (ECL kit, NEN Life Science Products, Inc.) and exposure to XAR film (Eastman Kodak Co.).

### Densitometric Analysis of Northern and Western Blots

Northern and western films as well as stained membranes and pictures of ethidium bromide-stained gels were digitally scanned and analyzed by densitometry. Bands were manually selected to obtain numeric values for band density (ImageJ program, NIH, public domain). Experimental values were corrected for loading before making experiment calculations.

### Statistical Analyses

Cell yields of pTT-treated fibroblasts were compared to diluent using 2-way ANOVA and a total of four different experiments, to identify significant difference between treatment groups as a function of cell number and time. The parallel MTS assay also utilized the 2-way ANOVA, to compare changes in OD at 492 nm as a function of time and treatment modality.

The hydrogen peroxide oxidative challenge assay data were generated with the combined results of four separate experiments. The averages for each treatment condition and timepoint studied (8, 24, 48, and 72 hours) were used to calculate the percent of adherent H₂O₂ treated cells relative to diluent-treated cells. Changes in cell yield were determined as a function of time using General Linear Model with repeated measure analysis (SPSS Version 10).

Error bars on all graphs are standard error of the mean. Because the cells used in this study are primary cells derived from different donors, the variability is much greater than found in established cell lines. Use of standard error of the mean adequately reflects mean deviation of measurements in each assay.

### pTT does not Modulate mRNA Levels of the Antioxidant Enzymes Studied

In order to determine whether pTT would affect the expression of antioxidant enzymes SOD1, SOD2, CAT and GPX, fibroblasts from a single donor for each experiment were cultured as described in Methods above and treated 24 hours after plating with either 100 μM pTT or diluent alone as a control. Cells were harvested for RNA at 8, 16, 24, 32, 48, and 72 hours after addition of pTT or diluent for most donors.

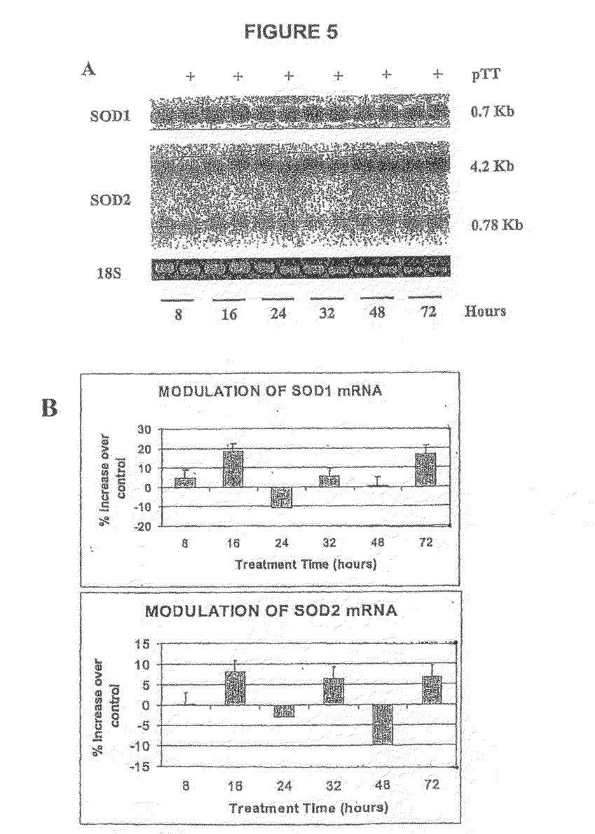

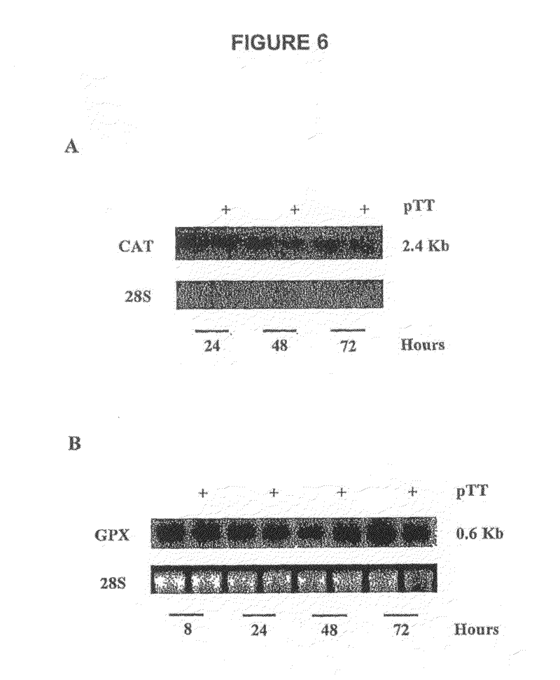

FIG. 5 shows representative Northern blots for SOD1 and SOD2 displaying no difference between pTT- and diluent-treated cells. Two of several reported mitochondrial Mn-dependent superoxide dismutase (SOD2) polymorphic transcripts were consistently detected by northern blot, at 4.2 kb and approximately 1 kb.^{118,182} Band intensity analysis from three different experiments suggest no consistent pattern of modulation for SOD1 or SOD2. CAT and GPX mRNA also remain unchanged by pTT treatment as compared to diluent-treated cells (FIG. 6). While CAT mRNA at 48 hours appears to be decreased in the presence of pTT compared to diluent, this was not consistently observed.

### pTT Modulates Mitochondrial Superoxide Dismutase Protein Levels

Cells were treated with pTT or diluent control medium as described above, and harvested for protein at 8, 16, 24, 32, 48, and 72 hours after addition of pTT or diluent for most donors.

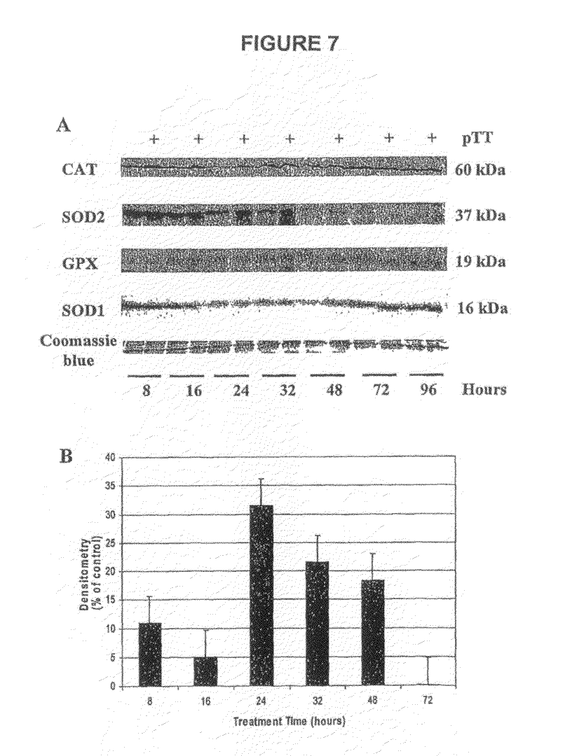

Only SOD2 protein shows modulation by pTT treatment, as shown in FIG. 7. This figure shows protein levels of all the enzymes studied from one representative donor. SOD2 protein levels were higher, as compared to diluent SOD2 protein levels, as early as 8 hours, reaching maximal 31% (±20%) relative induction at 24 hours and persisting through 48 hours.

### pTT Modulates Cell Growth but does not Decrease Viability

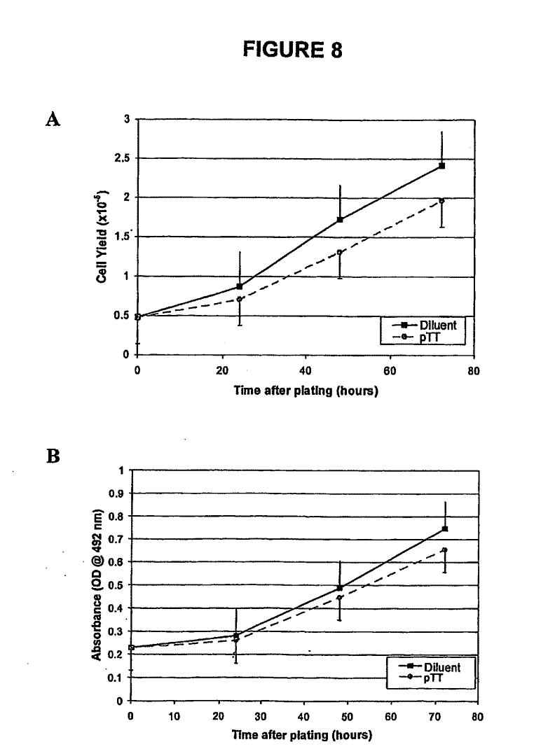

Fibroblasts plated at the same cell densities were treated the following day with either 100 μM pTT or diluent control. Cells were harvested by trypsinization after 24, 48 and 72 hours, without further feeding or re-treatment with pTT. Results of parallel studies measuring cell yields and the MTS viability assay in the same four donors are shown in FIG. 8. Panel A shows that the average rate of fibroblast growth during pTT treatment is significantly decreased compared to diluent-treated cell growth (2-way ANOVA for significant difference in cell yield over time, p=0.0079). However, the MTS assay (Panel B) shows that there is no significant decrease in cell viability (2-way ANOVA for significant difference in formazan absorbance over time, p=0.2588), suggesting that the decreased cell yields are more likely due to decreased cell growth rather than cell death. This is consistent with previous studies showing growth retardation in human dermal fibroblasts by 48 hours when stimulated with 50 μM to 150 μM pTT, as measured by Coulter counter cell yields.¹⁴² When considered in terms of reductive activity per cell, i.e. MTS absorbance per Coulter-counted cell, there was a significant increase in mitochondrial reductive activity per cell during pTT treatment (data not shown as this is merely a calculation of data from Panel B divided by Panel A, analysis by 2-way ANOVA for significance of differences in MTT values as a function of treatment group and time, p=0.0093).

### pTT Stimulates Resistance to Hydrogen Peroxide

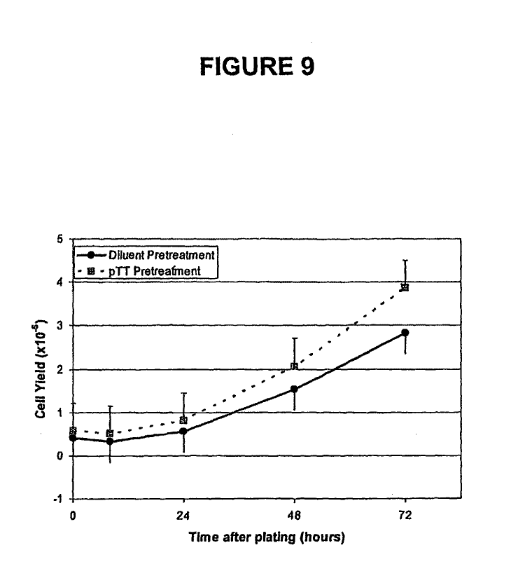

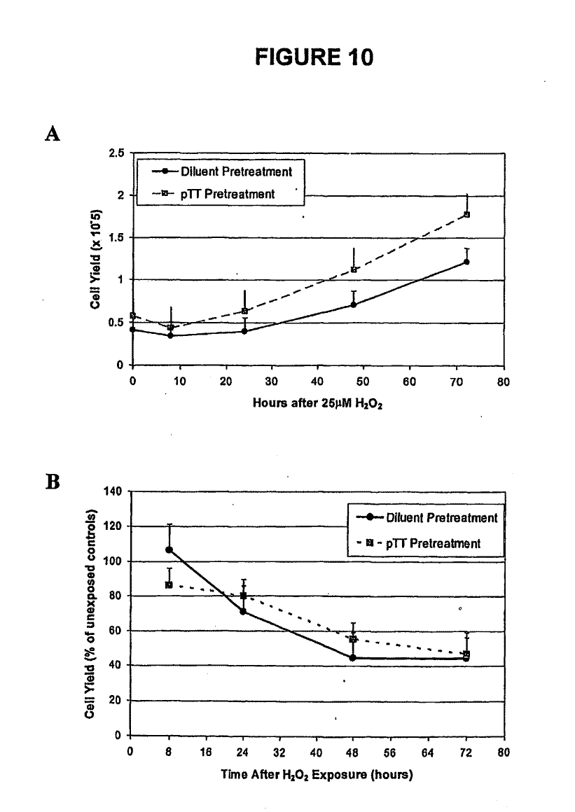

After 72 hours of pretreatment with either 100 μM pTT or diluent control, the same number of cells were replated with fresh 10% CS DMEM lacking pTT. The following day they were exposed to a dose of 25 μM H₂O₂ or DMEM control for 1 hour, and cell yields were determined at 8, 24, 48 and 72 hours following the H₂O₂ treatment. The growth of controls for each pretreatment group is shown in FIG. 9. Pretreatment with pTT significantly stimulated growth after replating in regular medium as compared to diluent-pretreated cell growth 1 (2-way ANOVA to compare cell yields over time as a function of pretreatment group, p=0.0006). Because of the differences in growth between the two H₂O₂ control groups, cell yields of H₂O₂ treated cultures were analyzed both by gross cell yields and as a percentage of control yields (FIG. 10). Panel A in FIG. 10 shows that pTT pretreatment for 72 hours results in higher cell yields over time following exposure to H₂O₂, as compared to diluent pretreated cells (2-way ANOVA for difference in cell yields over time, p=0.0008). Panel B cell yields, expressed as a percentage of respective H₂O₂ controls, shows a significant difference in cell yields in the pTT-pretreatment group at 24 and 48 hours as compared to diluent-pretreatment group. (General Linear Model (GLM) p=0.05, 2-way ANOVA p=0.93).

### Modulation of Mitochondrial Superoxide Dismutase Protein

There are no previous studies showing that mimicking DNA damage or telomere disruption stimulates antioxidant defense. In this study pTT, a dinucleotide with homology to a third of the telomere overhang sequence TTAGGG, stimulated an increase in the mitochondrial enzyme superoxide dismutase (SOD2) at the protein level, but not at the message level, as compared to diluent treatment. Because enzyme activity has been found to correlate with protein levels¹⁰⁴ this finding is suggestive of functional enhancement of antioxidant defense in mitochondria. Multiple studies show that an increase in SOD confers protection against oxidative damage from exogenous and endogenous ROS^{7,10,11,183,184} and increases the lifespan of C. elegans.¹³⁴ SOD neutralizes direct O₂.⁻ damage to cellular structures and, perhaps more importantly, reduces the amount of O₂.⁻ available to contribute to the generation of much more reactive and harmful species such as hydroxyl radical (OH.) and peroxynitrite (ONOO—).¹⁸⁵ In a recent study, SOD2+/− mice, which displayed 50% of normal SOD2 activity in all tissues, had higher amounts of 8-oxo-dG in nuclear DNA and a greater incidence of mice with tumors as compared to wild-type control mice,¹⁸⁶ suggesting that mitochondrial SOD2 plays an important role in preventing carcinogenesis.

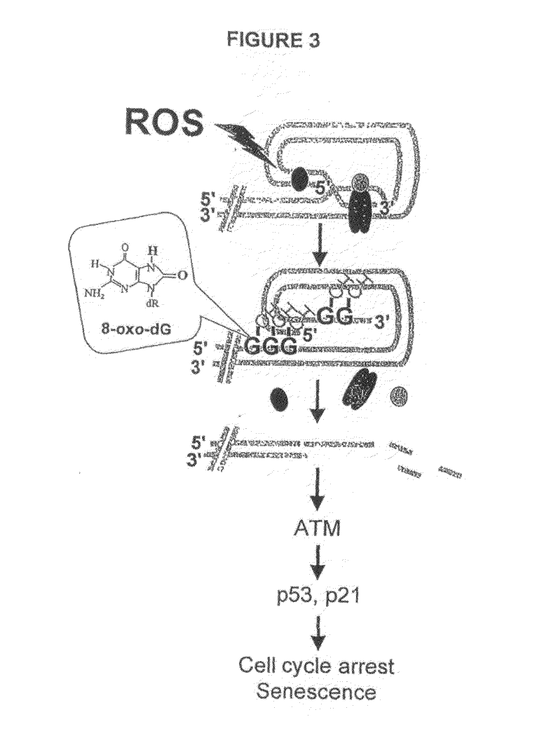

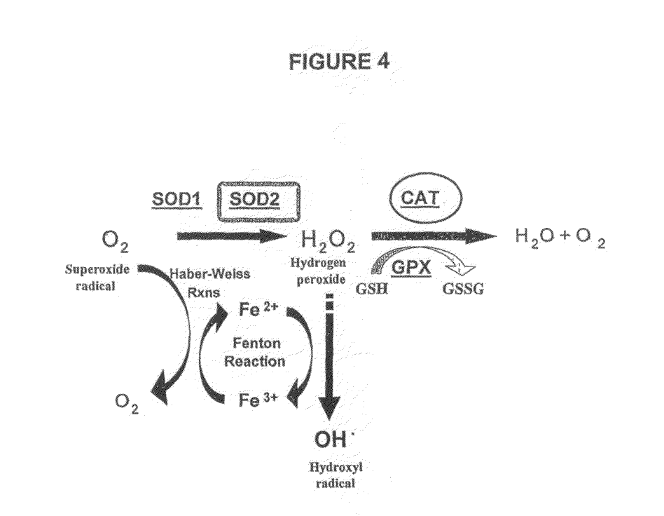

SOD2 mRNA was not consistently increased by treatment with pTT. The increased SOD2 protein levels without mRNA induction (FIGS. 3 & 5) might be attributable to increased protein stability,¹⁸⁷ an effect of pTT seen in previous studies on other cellular proteins such as p53¹⁴² and tyrosinase (unpublished data). Increased SOD2 protein stability was reported in WI38 human fibroblasts following gamma irradiation.¹⁸⁸ These studies support the possibility of a post-translational SOD2 response to mimicked DNA damage in fibroblasts treated with pTT.

Other studies show that absence of measured induction in mRNA, protein or activity of SOD1, CAT and GPX does not rule out antioxidant adaptive defense. In their oxidative stress adaptation study, Wiese et al. did not observe increases in mRNA or protein levels of CAT, GPX, SOD1 or SOD2, despite their resistance to cell killing or cell cycle arrest following toxic H₂O₂ treatment.¹⁴³ Stralin and Marklund exposed two fibroblast lines to several oxidant stressors for up to four days, yet detected less than two-fold induction of SOD2 activity throughout the investigation, and no effect on SOD1 activity.¹⁵⁷ Hardmeier et al. measured increased SOD and CAT enzyme activities in radiation-resistant mice within 15 minutes of whole-body X-ray irradiation, without measuring changes in enzyme transcription.¹⁵⁴ A study of cardioprotective modulation of SOD2 following ischemia-reperfusion in rats also supports that antioxidant defense responses, independent of transcriptional or translational modulation, is possible; Yamashita et al. measured a biphasic increase in SOD2 activity 30 minutes after intensive exercise without an increase in protein levels, and then another increase in activity at 48 hours with increased protein levels, all changes normalizing by 72 hours.¹⁸⁹

A large increase in SOD without a concomitant increase in H₂O₂-neutralizing enzymes CAT and GPX would, in fact, be harmful due to an imbalance in the fibroblasts' ability to cope with the greater levels of H₂O₂ produced by SODs. Xing et al. have shown in transgenic mice overexpressing SOD1 that moderate activity of SOD1 is protective, but high activity is toxic, creating more H₂O₂ than cells can neutralize.¹¹⁶

It is also possible that while this investigation was limited to SOD1, SOD2, CAT, and GPX, studies of other AOE might have detected further modulations, as in the study by O'Brien et al., who measured protective upregulation of glutathione reductase and glucose-6-phosphate dehydrogenase against acetaminophen toxicity in rat hepatic cells while SOD, CAT and GPX activities were decreased.¹⁹⁰

And finally, lack of modulation of AOE following pTT treatment could occur simply because pTT treatment is not toxic and therefore does not stimulate this kind of adaptive stress response. AOE are known to respond at the transcriptional and translational level to varied stressors such as direct oxidants, ischemia-reperfusion, cytokines, heat and cold stress,⁴ but it is conceivable that they only exhibit modulation of baseline mRNA or protein levels above a certain degree of physiologic stress.

It is interesting to note that SOD2 mRNA levels of both diluent and pTT treated cells increased with time while protein levels appeared to decrease; while this inverse trend was not always observed, it may reflect an increase in mitochondrial ROS levels causing utilization and degradation of SOD2 protein, or a change in SOD2 utilization accompanying cell growth and/or increased cell density in culture. Such changes in SOD2 during cell culture are reported in other cell types. An increase in SOD2 enzyme activity with length of culture time has been described in normal hamster kidney cells,¹⁹¹ and in melanoma cell lines the amount and activity of SOD2 protein increases with proliferation and differentiation.¹¹⁰ In a study of a plant SOD2 found to be functionally homologous to eukaryotic SOD2,¹⁹² induction of SOD2 correlated with stress conditions and sugar metabolism, specifically increasing with increases in cytochrome oxidase activity in the mitochondrial electron transport chain.¹⁹³ Thus, it is conceivable that the baseline increase in SOD2 mRNA while protein levels decrease in diluent-treated fibroblasts is a response to changes in nutrients in the culture medium, or responses to increasing cell density and proliferation with time.

### Thymidine Dinucleotide Stimulates Resistance to Oxidative Stress

The H₂O₂ oxidative challenge assay (FIG. 10) and the MTS viability assay (FIG. 8) show that pTT pretreatment stimulates adaptive resistance to oxidative stress. During pTT treatment growth is decreased relative to the diluent-treated control cells, consistent with previous studies of pTT showing p53 and p21-mediated cell cycle inhibition,¹⁴² but cell yields are increased after an oxidative challenge relative to pretreatment control cell yields. This can be interpreted as stimulation during pTT treatment occurring along with cell cycle inhibition, and subsequent adaptive resistance to oxidative stress.

Although cells observed under a microscope after the oxidative challenge showed no obvious cell death in either pretreatment group, early apoptosis cannot be ruled out in the pTT-pretreated group, which displayed lower cell yields than in the diluent-pretreated group at 8 hours after H₂O₂ treatment. However, the 24 and 48 hour timepoints reflect enhanced resistance to H₂O₂ in pTT-pretreated cells compared to diluent-pretreated cells. 10% higher relative cell yields were observed in pTT-pretreated cells at 24 and 48 hours after exposure to H₂O₂ compared to diluent-pretreated cells. By 72 hours the relative cell yields of diluent-pretreated controls was similar to pTT-pretreated cells, reflecting recovery from H₂O₂ in both pretreatment groups rather than irreversible toxicity.

Decreased growth rate in diluent-pretreated fibroblasts suggests oxidative stress sufficient to induce cell cycle arrest. It is well-known that oxidative stress triggers cell cycle checkpoints, especially at G₁ and G₂.¹⁹⁴ Oxidative stress-induced growth arrest in normal human fibroblasts has been shown to be mediated by ATM.¹⁹⁵ ATM was named for the DNA repair deficiency disease ataxia telangiectasia (AT) in which it was discovered, and was found to play an important role in initiating DNA damage signaling upstream of p53.¹⁹⁵

### Antioxidant Defense Responses After Telomere Homolog Oligonucleotide Stimulation

This study indicated that DNA damage responses induced by fibroblast treatment with pTT stimulates SOD2 protein induction and cellular resistance to oxidative stress without prior exposure to oxidants or irradiation as in the other studies of adaptive responses cited here.^{143,145,146,149,152,154} It has been proposed that SOD2 participates in signal transduction not only by neutralizing superoxides and preventing apoptosis,^{10,184,196} but also by serving as a source of H₂O₂ that leads to H₂O₂-mediated MAPK mitogenesis.¹⁹⁷ SOD2 is the only antioxidant enzyme to be upregulated by TNF-α, a stress/inflammation cytokine.¹⁹⁸ The modulation of SOD2 by pTT suggests that SOD2 participates in adaptive DNA damage signaling responses. In the presence of pTT, SOD2 may serve both as an AOE and a signaling molecule.

At the time of the H₂O₂ challenge, oxidative stress resistance was higher in the pTT-pretreated cells, which can be interpreted as reflecting lower intracellular levels of ROS than in diluent-pretreated cells. It is reported that low levels of H₂O₂ stimulate growth via the Erk/MAPK pathway, while slightly higher doses trigger ATM- and p53-mediated transient growth arrest.¹⁴³ However, it is possible that during the pTT pretreatment phase, intracellular ROS is transiently higher in pTT-pretreated cells, at least in the mitochondria, leading to the increased MTS assay absorbance per cell and the induction of SOD2 protein. Indeed, the MTS assay has been used to measure changes in the mitochondrial dehydrogenase NADH ubiquinone in the electron transport chain,¹⁸⁰ the major source of intracellular ROS.¹⁹⁹ A study by Berridge et al. also identified outer mitochondrial membrane and cytoplasmic NADH and NADPH oxidases as sources of MTT reduction.²⁰⁰ Thus, the increased MTS absorbance readings in fibroblasts treated with pTT reflect not only that the cells are viable, but also that they may be producing ROS through NADH or NADPH oxidases in mitochondria and/or the cytoplasm. A transient increase in ROS, along with increased SOD2, could stimulate adaptive resistance to subsequent oxidants such as H₂O₂. Intracellular ROS levels during pTT treatment were therefore measured, and the results support this interpretation. This data is presented below.

### Example 2

### Evidence of Redox Signaling in Response to Stimulation with Telomere Homolog Oligonucleotides

As previously discussed, telomeres are sensitive targets for oxidative DNA damage due to the richness of guanine residues in the telomeric repeat sequence and the decreased efficiency of repair to telomeric DNA.⁹¹ Hyperoxia has been shown in vitro to lead to telomere shortening and cell senescence in fibroblasts.¹² Furthermore, it has been shown that 8-oxo-dG, a major form of oxidative DNA damage, disrupts binding of telomeric proteins TRF1 and TRF2, which help to maintain the t loop structure and prevent telomere degradation.¹⁷ It is now also accepted that ROS, depending on their levels, are not only associated with cell damage leading to apoptosis or senescence but also are necessary for normal cellular signaling.^{4,201} Because T-oligo treatment stimulates many major DNA damage responses in multiple cell types including human primary fibroblasts,²⁰² and telomeres appear to be particularly vulnerable to oxidative DNA damage,⁹¹ it is reasonable to hypothesize that T-oligo treatment can stimulate adaptive signaling to protect against oxidative DNA damage. Since cell cycle arrest, apoptosis and senescence in response to genotoxic stimuli have been shown to involve active production of ROS for signal transduction, ¹⁵³′¹⁶⁹⁴⁷¹ it is also reasonable to hypothesize that T-oligos modulate intracellular ROS.

### Reactive Oxygen Species in Signal Transduction

### ROS Modulation of signal Transduction Pathways

It has begun to be appreciated that, at least for aerobic organisms, ROS are ubiquitous and even necessary for survival, as signaling molecules.^{4,81,201} ROS may modify proteins at specific amino acid residues such as cysteines and histidines, transiently changing their function rather than damaging the protein.^{4,203} ROS can decrease or enhance the ability of transcription factors to bind to DNA or other proteins, modulating protein activity and gene expression much like phosphorylation and dephosphorylation.^{4,204} In fact, phosphorylation/dephosphorylation of proteins may itself be modulated by ROS; ROS are thought to inactivate protein tyrosine phosphatases by modification of essential cysteine residues at the active site.^{4,205} Altered phosphorylation or modification of active binding sites by ROS are also thought to induce the modulation of redox-sensitive transcription factors such as NFKB, APE/Ref-1, SP1, Nrf2 and AP-1,^{206,207} as well as the tumor suppressor p53.^{4,203} One of the most well-characterized and accepted examples of ROS signaling is nitric oxide production by nitric oxide synthase in endothelial cells to regulate vascular tone.⁴ Thus, it appears that cells have evolved to utilize oxygen for oxidative modifications and the active production of ROS to effect appropriate cellular signals and responses.²⁰⁸

### NADH and NADPH Oxidases in Fibroblasts

ROS are produced within cells in many putative locations and in varying amounts by enzymes utilizing molecules such as nicotinamide adenine dinucleotide (NADH), nicotinamide adenine dinucleotide phosphate (NADPH) or other electron-carrying substrates such as flavin adenine dinucleotide (FADH or FADH₂), and their effects are dependent upon the degree of diffusion from their source and overall reactivity.^{4,183,208} NADH, NADPH and FADH are cofactors for redox reactions mediated by a family of flavoproteins, enzymes that utilize a flavin group (derived from the vitamin riboflavin) to either transfer electrons to other molecules, as in the four complexes of the mitochondrial electron transport chain, or to independently produce superoxide anion (O₂.⁻).^{199,209,210} This phenomenon was first described in neutrophils, which produce a bactericidal “oxidative burst” of O₂.⁻ through a membrane-associated NADPH oxidase enzyme system consisting of multiple subunits.^{204,211}

A similar plasma membrane-associated NADPH oxidase system has recently been identified in fibroblasts that produces O_{2.}⁻. The system spontaneously dismutates or reacts with other molecules to produce other ROS such as H₂O₂.²⁰⁹ H₂O₂ is produced on the order of 10⁻¹⁵ to 10⁻¹⁴ moles per cell,²¹² (approximately a third of that produced by phagocytic cells)⁴ within a second after fibroblast membranes in vitro are disrupted, demonstrating an ability to respond rapidly to membrane disruption that occurs during invasion by bacteria as well as to membrane changes during ligand binding.²¹³ NADH oxidases and NADPH oxidases in fibroblasts have been found to produce ROS in response to cytokines such as TGF-β1 and PDGF-BB,^{8,213,214} as well as TNF-α, a known inducer of SOD2, which can neutralize O₂.⁻ and produce H₂O₂, leading to other downstream signaling.¹⁹⁷

It is well-known that the major source of intracellular ROS in eukaryotes is mitochondria.¹⁹⁹ The mitochondrial electron chain consists of four cytochrome enzyme complexes that create a proton gradient in the intermembrane space by pumping protons (H⁺) from the matrix across the inner mitochondrial membrane as electrons are passed from Complex I or II progressively to Complex IV.¹⁹⁹ The proton gradient formed ultimately drives production of ATP from ADP by the inner mitochondrial membrane enzyme F1-F0ATPase.¹⁹⁹ Complex I and III are implicated in the production of ROS.²¹⁵ This production of ROS is thought to be regulatable by the amount of intracellular oxygen; for example, an NADH oxidoreductase in Complex I was recently reported to produce more superoxide in response to an oxidative shift in the mitochondrial redox status, as determined by oxidized glutathione levels.^{216,217} Production of H⁺ and unwanted ROS is also proposed to be regulated by a family of enzymes called uncoupling proteins (UCP) in the inner membrane, which pump H⁺ back into the mitochondrial matrix space.^{199,218} Mitochondrial ROS not only contribute to oxidative mitochondrial DNA damage, but are also thought to trigger cytoplasmic H₂O₂ signaling by diffusion²¹⁷ and even lead to nuclear DNA damage.²¹⁹

### ROS in DNA Damage Signaling

Evidence for a link between DNA damage and active ROS production was described in recent work on p53-induced genes (PIGs). In 1996, Johnson'et al. published evidence that ROS serve as p53-dependent mediators of apoptosis.¹⁷¹ The following year, Polyak et al. described a family of PIGs, many of which are redox-relevant genes. They found that induction of p53 corresponds to induction of an NADPH quinine oxidoreductase, PIG3, that causes a rise in ROS and apoptosis preventable by antioxidant treatment or dominant negative p53 (p53DN),¹⁶⁹ Macip et al. reported that p21(Waf1/Cip1/Sdi1) stimulates increased intracellular ROS and senescence in normal human fibroblasts independent of p53, PCNA or p16INK4a, and that reducing ROS levels using the antioxidant N-acetyl-L-cysteine prevented p21-mediated senescence.¹⁷⁰ p53-dependent induction of p21 leading to cell cycle arrest in G₀/G₁ protects against hyperoxia-induced DNA damage, presumably because during cell cycle arrest DNA is not unfolded for replication and is therefore physically less vulnerable to ROS modifications.²²⁰

Recent studies suggest that DNA repair is directly regulated by ROS, through activation of APE as reported in HeLa and WI 38 fibroblasts.¹⁴⁹ Other DNA repair enzymes such as xeroderma pigmentosum proteins,²⁰⁷ OGG1 in base excision repair (BER),²²¹ and NTH1 (human endonuclease III homolog)²²² are reported to have binding sites on their promoters for redox-sensitive transcription factors such as AP1, Sp2, Nrf2, and p53.²⁰⁷

The ERK/MAPK superfamily of enzymes, which include the small GTP-binding proteins Ras and Rac, is a source of intracellular ROS²²³ that upregulate DNA repair.²⁰⁷ Cho et al. reported that ROS generated by NADPH oxidase in NIH3T3 fibroblasts increased DNA repair efficiency of UV- and cisplatin-damaged plasmids through a Ras/phosphatidylinositol 3-kinase (PI3K)/Rac1/NADPH oxidase-dependent pathway.²⁰⁷ The ERK/MAPK family members are induced by multiple extracellular stimuli such as growth factors, UV, heat shock, hyperoxia and hypoxia, and their activation has been found to upregulate telomerase in hypoxic solid tumor cells.^{224,225}

### T-Oligo Treatment as a Model for DNA Damage

The study of DNA damage responses has helped to explain how cells and organisms survive noxious stimuli and avoid carcinogenic transformation. It has been proposed that eukaryotes have evolved a DNA damage sensing system that overlaps with telomere repair and maintenance mechanisms, since telomeres are sensitive targets for DNA damage.^{91,94,95,202,226} DNA damage responses have been studied using multiple genotoxic stimuli, including ionizing radiation, UV, and treatment with chemical carcinogens and oxidants such as H₂O₂.^{88,93,149,150,227} Telomere-specific DNA damage has been investigated by treating cells with alkylating agents and H₂O₂,⁹¹ or by causing loop disruption using transfection of dominant-negative TRF2.⁷¹

However, DNA damage responses, including tumor suppression, can also be elicited without damaging DNA or disrupting the t loop.²⁰² G-rich telomere sequence oligonucleotides in solution have been treated with oxidating agents to induce oxidative DNA lesions and observe changes in the binding affinity of telomere proteins.^{17,94,226} Cells treated with linear single-stranded oligonucleotides homologous to the telomere 3′ overhang^{13,68,228} and plasmids containing the same sequences behave as if they sense actual DNA damage or telomere disruption, suggesting that there is a sequence-specific DNA damage response to telomere overhang exposure rather than a response to random DNA strand breaks.²²⁹ Observations using all of these models support the hypothesis that telomere DNA damage is associated with t loop disruption and the production of G-rich fragments from the degrading telomere 3′ overhang.^{71,95}

The Gilchrest group has used exogenous telomere homolog oligonucleotides (T-oligos) to show that human dermal fibroblasts and other cell types display the same responses as seen after DNA damage or TRF2 dysfunction. Eller et al. have demonstrated induction of ATM, p53, p21 and several other cell type-specific DNA damage responses including cell cycle arrest,^{13,142} senescence,^{68,72}, apoptosis,²²⁸ melanogenesis,^{173,175} enhanced DNA repair,^{68,72,142,172,230} and immune modulation,^{177,231} as a response to mimicking prolonged 3′ overhang exposure, without evidence of telomere shortening.^{13,228} Many or all of the responses appear to be mediated by p53¹⁷² or proteins with similar or cooperative functions, such as p73, p95/Nbs-1, and E2F1.¹³ It was determined that nuclear uptake of these oligonucleotides is enhanced significantly by 5′ phosphorylation in murine melanoma cells, and that oligonucleotides with greater homology to the telomere sequence are more effective in stimulating these responses.¹⁷⁵

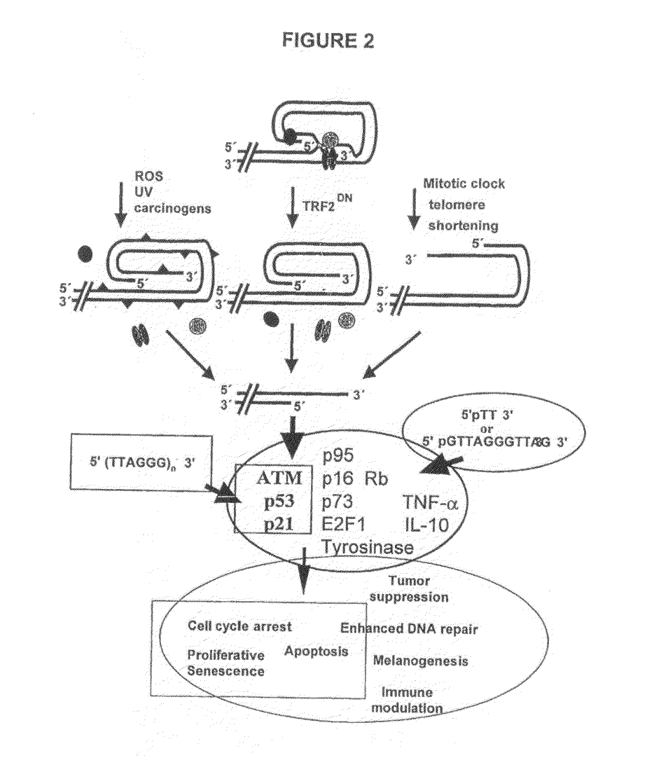

More recently, T-oligos have been used to treat tumors. Administration of TO reduced melanoma tumors in a SCID mouse model,¹⁷⁶ decreased the incidence of tumor formation in nude mice repeatedly exposed to solar simulated UV,¹⁷⁴ and initiate apoptosis and reduce tumor size in multiple epithelial tumors via modulation of ATM, p53, p95/Nbs-1, E2F1, and p73 as well as induction of proapoptotic protein Bax and phosphorylation of histone H2AX.²³² In addition, current work suggests that T-oligo effects are also mediated by transcriptional regulation by histone deacetylation,²³³ DNA damage recognition by telomere-associated poly(ADP-ribose) polymerases (PARPs) called tankyrases,²³⁴ and Werner protein's nuclease activity in cooperation with DNA damage-sensing protein DNA-dependent protein kinase (DNA-PK).²³⁵ See FIG. 2 for a summary of published responses to telomere loop disruption or damage.

Data was presented above demonstrating that T-oligos stimulate resistance to H₂O₂ treatment in human dermal fibroblasts. It was also found that SOD2 protein levels were increased as compared to diluent-treated fibroblasts. This suggests that T-oligos stimulate a protective, adaptive response to oxidative stress mediated at least in part by induction of SOD2, a major antioxidant and signal transduction molecule.¹⁹⁷

The present studies aim to further elucidate how T-oligos stimulate DNA damage responses, with the hypothesis that T-oligos modulate ROS production. The goals of this example were 1) to investigate the effect of T-oligos on reactive oxygen species (ROS) levels to help characterize redox responses to mimicked telomere disruption in human newborn fibroblasts; 2) to investigate the relationship of p53 induction and activation to modulation of ROS levels in human newborn fibroblasts and 3) to conduct dose response and time course studies to compare the effects of pTT to those of the 11-base T-oligo (TO) in human newborn fibroblast redox responses to mimicked exposure of the telomere 3′ overhang.

### Fibroblast Cell Culture

Normal newborn human dermal fibroblasts were cultured from foreskin specimens into DMEM supplemented with 10% CS as described above. Due to the large number of cells needed for time course experiments, and to minimize donor variability, in dichlorofluorescein diacetate FACS experiments cells from three different donors were combined.

R2F fibroblasts (a kind gift from J. Rheinwald, Harvard Medical School, Brigham and Women's Hospital) were obtained to study the involvement of p53 in ROS production. These human cells were retrovirally transduced to produce high levels of a dominant-negative p53 protein and hence have no functional p53.²³⁶ Matching wild-type p53 cells were used as controls. The culture medium consisted of 15% FBS in a 1:1 v:v mixture of DMEM and Ham's F12 medium. Otherwise, they were handled and seeded in the same manner as the primary foreskin fibroblasts.

### Chemicals

Hydrogen peroxide (30% w/w, with 0.5 ppm stannate and 1 pmm phosphorus as preservatives) was obtained from Sigma (USP grade, St. Louis, Mo.). The stock bottle was stored at 4° C. and all dilutions were made in DMEM immediately before use. 2′,7′-dichlorodihydrofluorescein diacetate (H₂DCFDA) was obtained in powder form (Molecular Probes, Inc., Eugene, Oreg.), dissolved in DMSO to a stock concentration of 1 mg/ml, aliquotted and stored under nitrogen at −20° C. The product was protected from light during handling and storage. Because the solution is less stable than powder, small batches of solution were made only as needed. Propidium iodide was obtained from Sigma (St. Louis, Mo.). Diphenyliodonium chloride (DPI) was obtained from A.G. Scientific, Inc. (San Diego, Calif.). DPI powder was dissolved in DMSO to a stock concentration of 5 mg/ml, aliquotted and frozen at −20° C. until use. For the senescence-associated-β-galactosidase assay staining solution, X-gal (5-bromo-4-chloro-3-indolyl-β-D-galactopyranoside) was dissolved in dimethylformamide and added to a buffer immediately before use for a final concentration of 1 mg/ml. The major buffer ingredient, citric acid/Na phosphate, was adjusted to pH 6.0.

### Oligonucleotide Preparation and Cell Treatment

Purified lyophilized oligonucleotides were obtained and prepared for cell treatment as described above. All treatments involved a single stimulation with T-oligo, after which cells were harvested at various times without medium changes or addition of more T-oligo. Treatment doses were 100 μM pTT and 40 μM TO except in dose-response experiments; these concentrations were determined in previous experiments to be optimal for measuring DNA damage responses using these T-oligos.^{142,150,228}

Complementary and scrambled oligonucleotides were chosen for each T-oligo. pAA and pCC were used as controls for pTT, and the complementary 11-base pCTAACCCTAAC (TOC1, SEQ ID NO: 22) and a scrambled sequence pGATCGATCGAT (TOC2, SEQ ID NO: 23) were used as controls for the T-oligo TO.

### Extracellular Hydrogen Peroxide Generation Assay

This assay was described by Ruch et al. for measurement of H₂O₂ production by macrophages and neutrophils⁹³ and modified for cultured cytokine-stimulated human lung fibroblasts by Thannickal et al.⁹⁴ Briefly, it utilizes horseradish peroxidase (HRP) to catalyze H₂O₂-dependent dimerization of tyrosine in homovanillic acid (HVA), where the H₂O₂ is the extracellular fraction of H₂O₂ produced by stimulated cells. Fibroblasts were seeded at 0.5×10⁴ cells/cm² and treated the following day with diluent, 100 μM pTT, 100 μM pAA, 40 μM TO or 40 μM TOC1 for two days. These doses were determined in previous studies to be the optimal effective doses to achieve p53 induction and cell cycle arrest in fibroblasts within the parameters used in these investigations (cell seeding density and time of treatment).^{13,142} An assay medium consisting of sterile Hanks' Balanced Salt Solution (HBSS), HRP (5 U/ml) and HVA (0.1 uM) was added to cells after removal of the T-oligo-supplemented medium. The assay medium was collected 30-60 minutes after incubation in 37° C. and 6% CO₂, when the reaction was stopped by changing the pH of the solution using NaOH-glycine (0.1 M glycine in 12 N NaOH). Each sample was fluorometrically analyzed by excitation of the dimerized tyrosine product at 323 nm with emission measured at 423 nm (Perkin-Elmer LS-5B Luminescence Spectrometer). The same cells were then harvested and counted by Coulter Counter. Fluorometry results were normalized to background. Using the cell count results, H₂O₂ production was expressed as a function of time and cell number (pmol/min/million cells).

### Dichlorofluorescein Diacetate (DCF) Assay

H₂DCFDA stock solution (1 mg/ml) was thawed and diluted in Hanks' Balanced Salt Solution, 1× liquid without phenol red (GIBCO Invitrogen, Carlsbad, Calif.), to a working concentration of 100 μM immediately before use. H₂DCFDA is converted by intracellular esterases to dichlorofiuorescein (DCF). When oxidized by intracellular ROS, it will fluoresce at 530 nm when excited by 480 nm light. Fibroblasts were treated for various times with T-oligos, diluent or control oligonucleotides, incubated for 30 minutes at 37° C. and 6% CO₂ with 100 μM DCF solution, harvested with EDTA and trypsin, and kept on ice shielded from light until FACScan analysis. All work involving DCF was conducted in minimal room light. Peaks on FACScan plots that shift to the right indicate greater fluorescence and increased ROS levels. For NADPH oxidase inhibitor studies using DPI,^{237,239} the DPI stock solution was added directly to DCF treatment solution to achieve a final DPI treatment concentration of 50 μM.

Propidium iodide (PI, Sigma) was used to stain nonviable cells for some experiments. PI stock solution (1 mg,/ml) was added to samples immediately after harvesting to achieve a final concentration of 2 μg/ml.

Hydrogen peroxide positive controls were used to rule out saturation of the DCF probe in the T-oligo dose response experiments. Fibroblasts were exposed to 1 mM, 5 mM and 10 mM H₂O₂ solutions in PBS for 15 minutes following DCF incubation for 30 minutes, harvested and analyzed as described above.

### Western Blot Analysis

Western blot analysis of proteins from cells treated and harvested in parallel with DCF time course studies was conducted as described above. Membranes were reacted with antibodies to total p53/DO-1 (1:1000 dilution, Oncogene Research Products, Cambridge, Mass.), anti-phoso-p53 (Ser-15) (1:1000 dilution, Cell Signaling Technology, Beverly, Mass.), and p21/Cip1/Waf1 (1:500 dilution, Transduction Laboratories, Lexington, Ky.), followed by appropriate secondary antibodies diluted 1:2000.

### Senescence-Associated β-Galactosidase (SA-β-Gal) Assay

Subconfluent senescent fibroblasts were found by Dimri et al. to stain blue in an assay that measures β-galactosidase activity at pH 6.0.²⁴⁰ As per their protocol, cells were fixed with a 2% formaldehyde and 0.2% glutaraldehyde solution, washed, and stained overnight in X-gal solution. The next day, 3-4 photographs were taken of representative fields in each plate under a 10× microscope objective using coded labels. The number of blue (senescent) cells in each photograph could then be determined in a blinded fashion. The SA-β-gal assay was performed in parallel with the DCF time-course experiments, using the same mixture of donor cells and seeding densities.

### Statistical Analyses

The horseradish peroxidase assay data was analyzed by one-way ANOVA. DCF assay FACScan results were analyzed using Cellquest software (Becton-Dickinson, Calif.). All peak positions on x-axis of the fluorescence histogram plot were determined visually (using the software) and recorded as a number. Peak position values were then compared using one-way ANOVA.

### T-Oligos Stimulate p53-Dependent NAD(P)H ROS Signaling

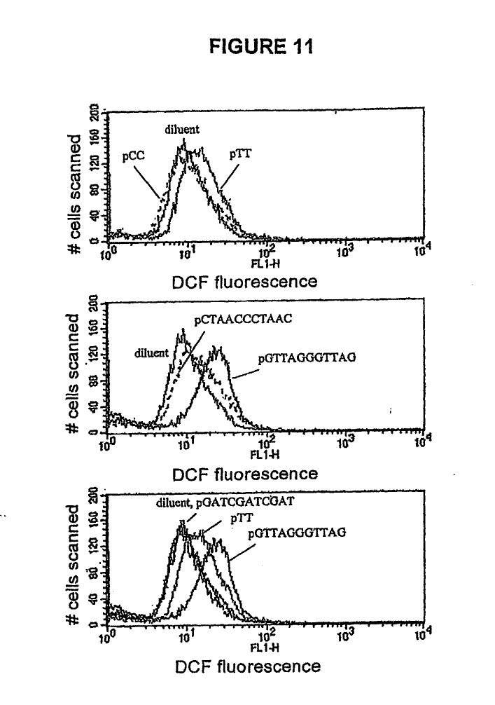

The dichlorofluorescein diacetate (DCF) assay showed that there is a significant increase in intracellular ROS levels during T-oligo treatment, as compared to diluent and control oligonucleotides (FIG. 11). Only pTT and TO stimulated a statistically significant increase in DCF fluorescence. pTT was compared to diluent and pCC controls by one-way ANOVA, yielding a p-value of 8.1×10⁻⁵ (n=11). Post-hoc analysis showed significant differences between pTT and diluent as well as pTT and pCC (both comparisons p<0.0004). TO compared to diluent and the control sequences TOC1, and TOC2 were significant (one-way ANOVA, p=0.01 for n=5). Post-hoc analysis showed that TO was significantly different from all the controls (p<0.02 or less), while the controls were not significantly different from each other.

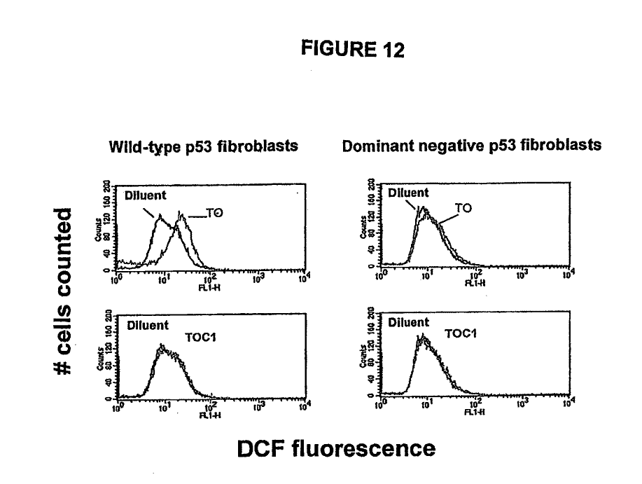

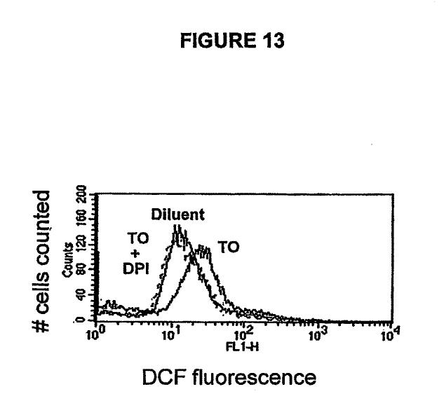

T-oligos did not stimulate increased ROS in p53 dominant negative R2F cells (p53DN) as they did in the matching wild-type (WT) cells (FIG. 12), demonstrating that the stimulated ROS production is p53-dependent. Because p53-dependent ROS production in fibroblasts has been attributed to NAD(P)H oxidase activity,¹⁶⁹ a specific NADPH oxidase inhibitor, diphenyliodonium chloride (DPI),²³⁹ was added to the DCF assay medium to determine the role of NADPH oxidases in T-oligo mediated ROS induction. FACScan analysis showed that DPI treatment consistently abrogated the increase in ROS stimulated by T-oligos (FIG. 13).

### Time Course Relationship of ROS, p53 and p21 Modulation by T-Oligos

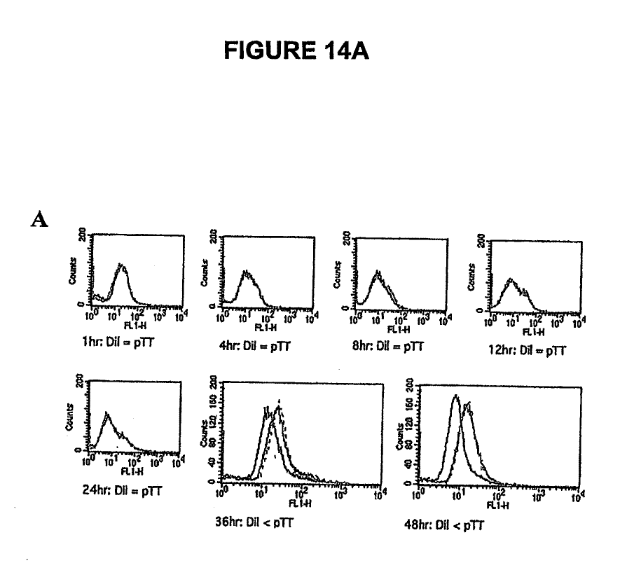

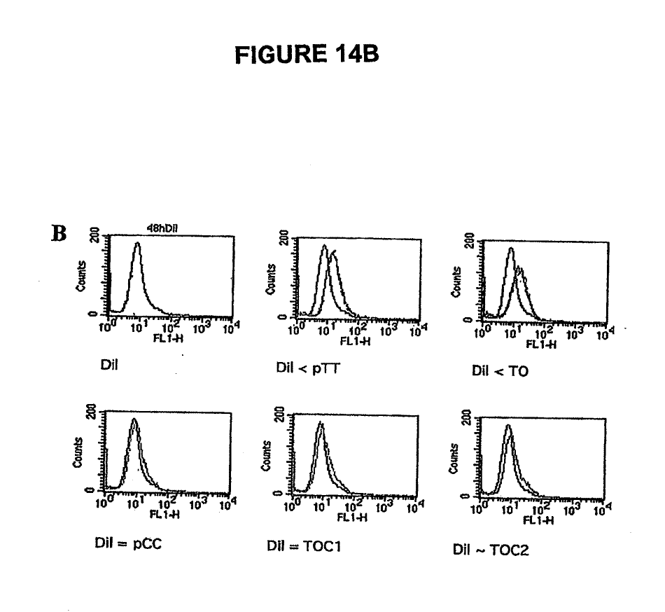

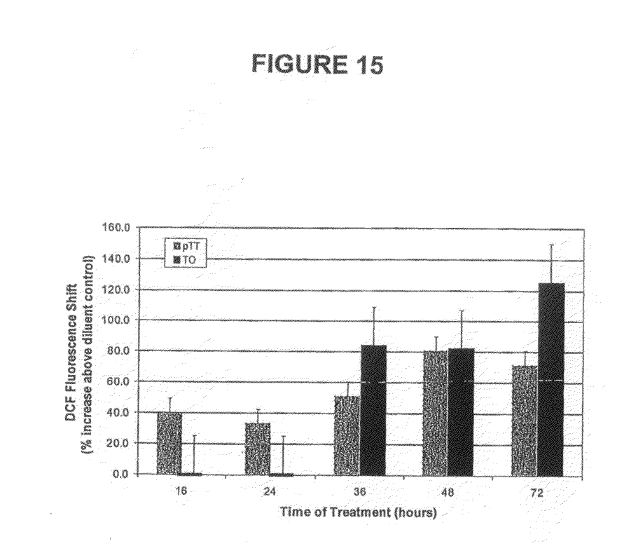

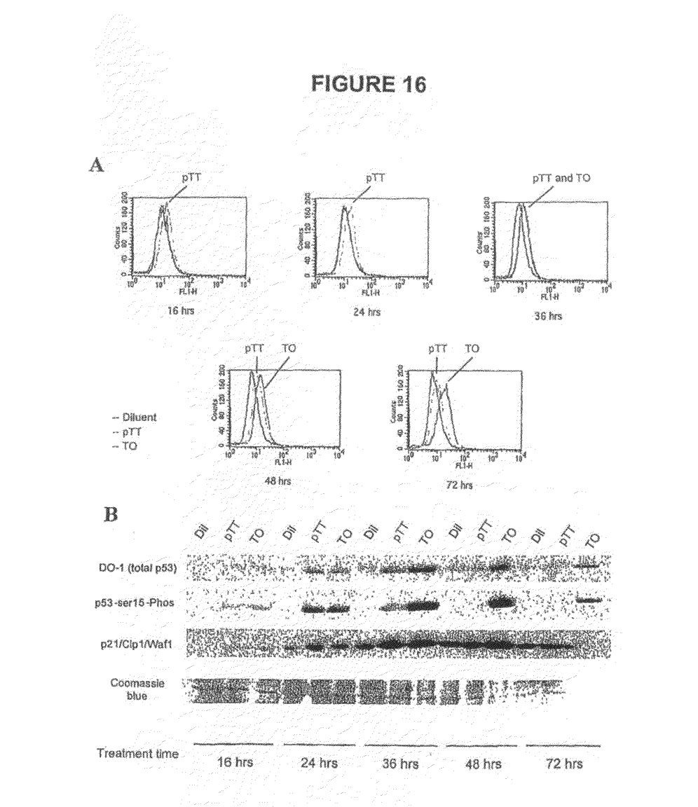

To further delineate the effects of T-oligos on ROS signaling, time course experiments were conducted to determine the onset of ROS stimulation and its relationship to induction and/or activation of p53 and p21, signaling events reported to occur either in response to elevated ROS,^{16,241} concurrently with ROS elevation,¹⁷¹ or preceding intracellular production of ROS.^{169,179} Although in some experiments pTT stimulated ROS at the same time as TO (FIG. 14), pTT-stimulated ROS were measured up to 20 hours earlier than TO, while all controls were similar to diluent-treated controls (FIG. 15). However, the maximum amount of DCF fluorescence in TO-treated cells was greater than ROS stimulated by pTT-treated cells, at a dose of 40 μM compared to 100 μM pTT (FIG. 15). FIG. 15 shows the time course and amount of ROS stimulation expressed as the percentage induction above diluent control baseline ROS levels. Increased ROS were measured at the same time or several hours after induction of total p53 protein, p53-serine-15, and p21 (FIG. 16). By 36-48 hours the response to TO was greater than to pTT according to all parameters measured (DO-1, phospho-p53-ser15, and p21 protein levels and DCF fluorescence), which was sustained through the 72 hour timepoint (FIG. 16). Note that the 72 hour lane was underloaded, as determined by Coomassie blue gel staining.

### The 11-Base T-Oligo has Greater Molar Efficacy for ROS Stimulation than pTT

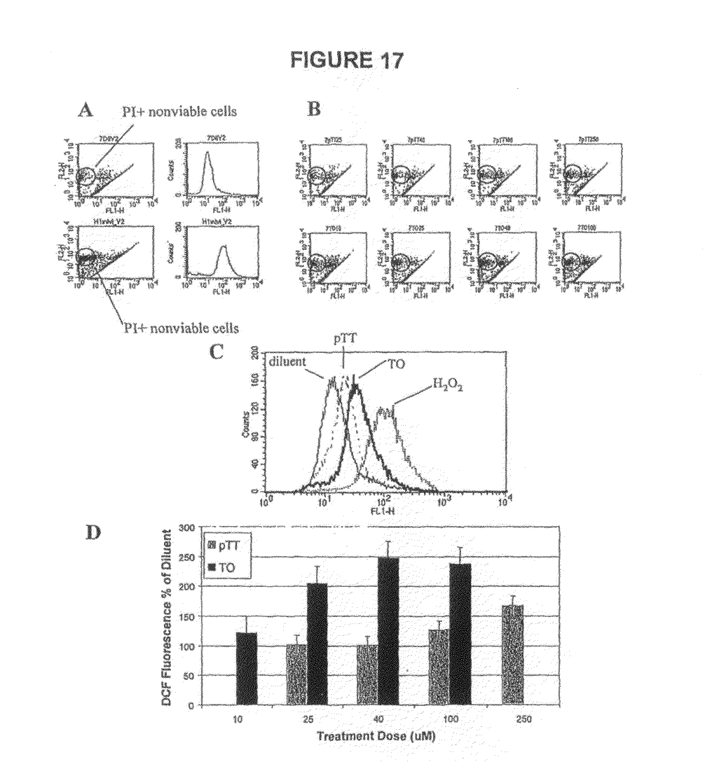

To better characterize the dose effects of different pTT and 11-base T-oligo doses, a dose response study was conducted. For each T-oligo the doses were 25-250% of the standard dose used (40 μM for TO and 100 μM for pTT). Because the effect of high doses of T-oligos on cell viability was unknown, propidium iodide (PI) staining was used to exclude nonviable cells because the DNA of nonviable cells take up the stain and fluoresce.²⁴² Panel A of FIG. 17 shows the PI fluorescence subset in cells treated with a toxic dose of H₂O₂ (1 mM) as a positive control for the DCF assay. PI fluorescence in all pTT and TO-treated cells were less than that induced by the positive control (Panel B). Panel C is a compilation of the maximum DCF peak shifts measured with each treatment: diluent, 250 μM pTT, 100 μM TO and 1 mM as the positive control. Although the two higher doses of TO stimulated similar levels of DCF fluorescence, this cannot be attributed to saturation of the DCF probe, since a positive control treatment of 1 mM H₂O₂ for 15 minutes stimulated a greater shift than any of the T-oligo treatments (Panel C). Panel D shows that within 72 hours of treatment the 11 mer stimulated up to 1.5 times more ROS production as measured by DCF fluorescence than pTT at the same dose. There is a significant difference in DCF fluorescence stimulated by the two treatments when the same doses were used (25, 40 and 100 μM) (2-way ANOVA, p=0.0038). Post-hoc analysis shows a significant difference between pTT and TO for each of the doses (p<0.03).

### Senescence is Not a Major Response to Limited T-Oligo Treatment

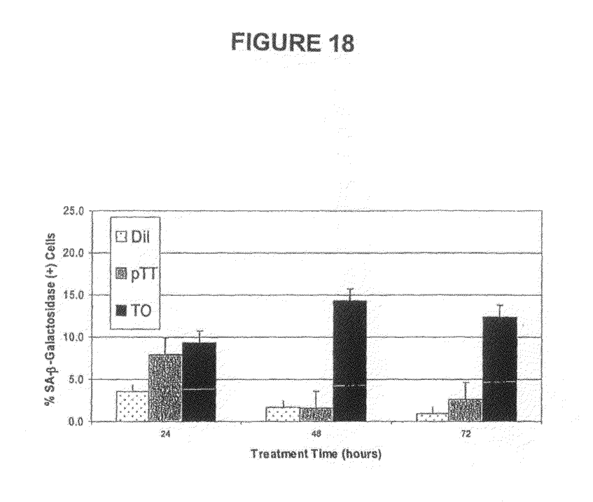

The SA-β-gal assay²⁴⁰ is now a well-accepted method for identifying senescent cells in culture, and was used by Li et al. to show that extended T-oligo treatment (one week treatment) induces senescence in over 60% of cultured human dermal fibroblasts.⁶⁸ The assay was therefore used in this investigation to determine whether shorter T-oligo treatment times of ≦72 hours induces senescence in the same cell type (FIG. 18). The assay was conducted in parallel with DCF time course assays to correlate levels of ROS, p53 and p21 with senescence, using the same cell donors. FIG. 18 shows a modest increase in TO-treated cells staining positive in the SA-β-gal assay, less than 15% throughout the 72 hours of treatment. This was found to be significant as compared to diluent- and pTT-treated cells (2-way ANOVA for the effect of treatment group over time, p<0.01, with post-hoc analysis identifying TO as significantly different). Less than 10% of cells treated with 100 μM pTT stained positively for SA-β-gal and this was not statistically different from the diluent-treated control in the ANOVA post-hoc analysis.

### T-Oligos Do Not Stimulate Detectable Extracellular H₂O₂ Production

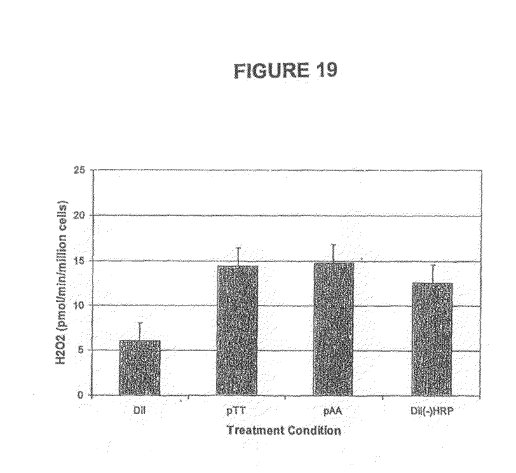

The horseradish peroxidase assay was used to determine whether extracellular H₂O₂ was increased as a result of T-oligos. Newborn fibroblasts were treated for 2 days with pTT, pAA and diluent control to assess extracellular H₂O₂ levels (as described above in Methods). FIG. 19, a representative experiment comparing diluent, pTT, pAA and the negative control using medium lacking horseradish peroxidase, shows that pretreatment with pTT does not yield extracellular H₂O₂. All values were comparable to control results using assay medium lacking HRP (p=0.78, one-way ANOVA).

### ROS Generation in Response to T-Oligo Stimulation

Several studies demonstrate that ROS are induced in fibroblasts in response to activation of p53 and induction of p21.^{169,171,243} The experiments using p53^{DN} R2F fibroblasts (FIG. 12) show that a lack of functional p53 abrogated the T-oligo-stimulated ROS, demonstrating p53-dependent ROS production, in agreement with previous findings. However, ROS production in response to telomere DNA damage or mimicked telomere damage has not previously been described.

This study strongly suggests that DNA damage responses induced by telomere overhang-homologous oligonucleotides stimulate production of intracellular ROS. TO was included in the study of ROS stimulation to observe the effect of a larger oligonucleotide with full telomere sequence homology. Only T-oligos (pTT and TO) stimulated ROS levels that were significantly different from those of diluent and oligonucleotide controls. Modulation of p53 and p21 proteins preceded the measured stimulation of ROS and paralleled the intensity of stimulation, in that induction and/or activation of these DNA damage response proteins were highest in TO-treated cells and ROS stimulation was also highest in TO-treated cells (FIGS. 16 and 17). Furthermore, the timing of p53 induction observed in this study is consistent with published reports of p53 induction within 8 hours of treatment with 40 μM of the 11-base T-oligo¹³ and other reports showing induction of ROS along with,¹⁷¹ or several hours after p53 protein overexpression.¹⁶⁹ In these and other studies it was repeatedly shown that the control oligonucleotides did not modulate p53 or p21.^{13,68,72,228} In this investigation, p21 levels were also elevated, as observed previously with T-oligo stimulation,^{68,72} although it does not prove that p21 is necessary for T-oligo-stimulated ROS in this system.

It is interesting to note that pTT often stimulated ROS earlier than TO (FIGS. 15 and 16). p53 and p21 induction followed this pattern in such donors, peaking earlier than TO induction of p53 and p21, although the TO-stimulated responses appear to last longer (FIG. 16). Possible explanations for the difference in time courses between pTT and TO include cellular uptake characteristics and possible differences in signaling mechanisms. A study of oligonucleotide transport into the myeloid cell line HL60 showed that the rate of uptake and maximum intracellular concentration is inversely proportional to the size of the oligonucleotide; an oligo(dT)₃ was taken up more quickly and to higher levels than larger oligonucleotides such as an oligo(dT)₁₅.²⁴⁴ This suggests that pTT is taken up more quickly than TO. Previous work with fluorescently-labeled oligonucleotides showed pTT accumulation predominantly in the cytoplasm, while a p9mer oligonucleotide appeared to accumulate more in the nucleus of S91 murine melanoma cells.¹⁷⁵ This may explain why a higher dose of pTT is needed to stimulate DNA damage responses than TO. Alternatively, the accumulation of pTT in the cytoplasm of cells might stimulate a slightly different pattern or time course of responses. Recent study of the mutated progeroid Werner's syndrome protein suggests that sensing telomere DNA damage involves Werner protein nuclease activity;²⁴⁵ since digestion of TO would yield thymidine dinucleotides, perhaps pTT treatment bypasses a nuclease digestion stage and initiates DNA damage responses faster. Clearly, further studies are needed to determine which, if any, of these possible explanations are involved in the differences between pTT and TO response time courses.

Because of convincing evidence that senescence can be initiated by p53, p21 and ROS as a DNA damage response,^{170,246}as well as a prolonged exposure to TO,^{68,72} the SA-β-gal assay conducted in this investigation was helpful to determine whether senescence was a major response to T-oligos in this study. FIG. 18 shows that the pTT treatment did not result in a significant number of SA-β-gal cells as compared to control treatments. However, TO stimulated senescence in up to 14% of treated cells within the study time frame, in a statistically significant manner as compared to diluent and pTT treatment over time (2-way ANOVA, p<0.01). This data are in agreement with the observation that TO stimulates p53 and ROS to a greater degree than does pTT (FIGS. 16-18). However, this result is much smaller than that observed with a longer course of stimulation,^{68,72} suggesting that the ROS produced in response to T-oligos do not initiate senescence as a major response.

### Identifying the Source of ROS Stimulated by T-Oligos

As discussed earlier, two major sources of intracellular ROS production are the mitochondrial electron transport chain and NADPH oxidases such as the fibroblast plasma membrane-associated NADPH oxidase. ^{199,204,209} While several studies have shown NADPH oxidase-mediated production of ROS in response to increased, levels of oncogenic Ras or Rac, members of the ERK/MAPK stress and mitogenic response pathway,^{207,247,248} production of p53-dependent ROS production by NADPH oxidases has not previously been described. In this study it, has been shown that both p53^{DN} and the flavoprotein inhibitor DPI completely and consistently abrogate the increase in ROS stimulated by T-oligos (FIGS. 12 and 13), suggesting that T-oligos stimulate ROS production through the activation of p53-dependent NAD(P)H oxidases.

The exact identity and cellular location of the enzyme(s) responsible for T-oligo-stimulated ROS induction remains to be elucidated. DPI is typically described as a specific NADPH oxidase inhibitor²⁴⁹ and has been used in other studies with the DCF assay to show involvement of NADPH oxidase in ROS production.^{213,250} More accurately, DPI is capable of binding and inhibiting flavoproteins in general.^{237,239} Flavoproteins include the NADH oxidase in mitochondrial cytochrome complex I, nitric oxide synthase, cytochrome P450 reductase, xanthine oxidase and sulfite reductase.²¹³ Among these, only NADPH oxidases and mitochondrial NADH oxidases have been repeatedly identified as potential sources of regulatable ROS.^{199,216,251} Further studies are needed to confirm the location of ROS production by NAD(P)H oxidases stimulated by T-oligos. The lack of extracellular H₂O₂ measured in the HRP assay (FIG. 19) suggests that it is an intracellular source of ROS that do not diffuse through the plasma membrane. Alternatively, it has been reported that this assay may underestimate the amount of H₂O₂ produced, so another assay or a longer incubation time may have yielded different results.²⁵²

### T-Oligo Increases Resistance of Human Ribroblasts to H₂O₂

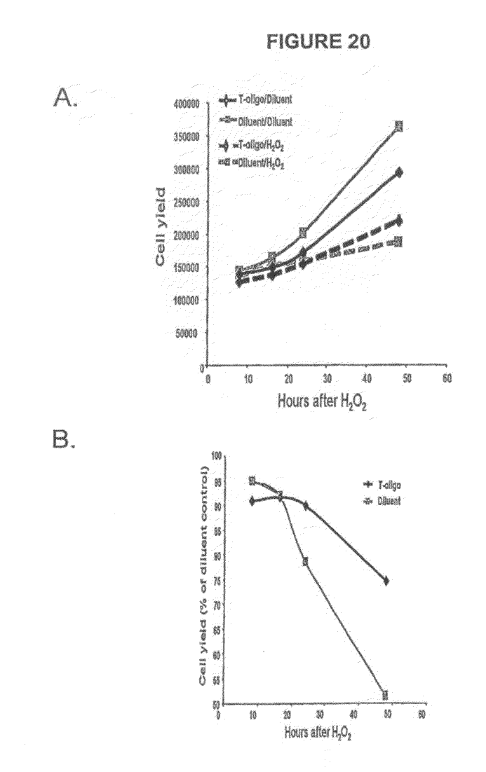

Newborn fibroblasts cells were plated in DMEM supplemented with 10% CS. Forty-eight hours after plating, cells were provided fresh medium. Twenty-four hours later, cells were provided fresh medium containing 40 μM of pGTTAGGGTTAG (abbreviated as TO, SEQ ID NO: 1) or diluent as a control. Seventy-two hours later, cells were harvested and replated in fresh medium lacking oligonucleotides. Twenty-four hours later, cells were provided fresh H₂O₂ (25 μM) or diluent for 1 hour and then provided fresh DMEM with 10% CS. Cell yield was then measured in TO-pre-treated cultures as well as control cultures. TO-pre-treated cells displayed increased resistance to H₂O₂ as measured by total cell yield (FIG. 20A). Furthermore, significantly higher number of T-oligo pre-treated cells in comparison to non-treated control (75% versus 52% respectively) survived the oxidative challenge by H₂O₂ (FIG. 20B).

### T-Oligo Upregulates the Level of Anti-Oxidant Enzymes at the Protein Level

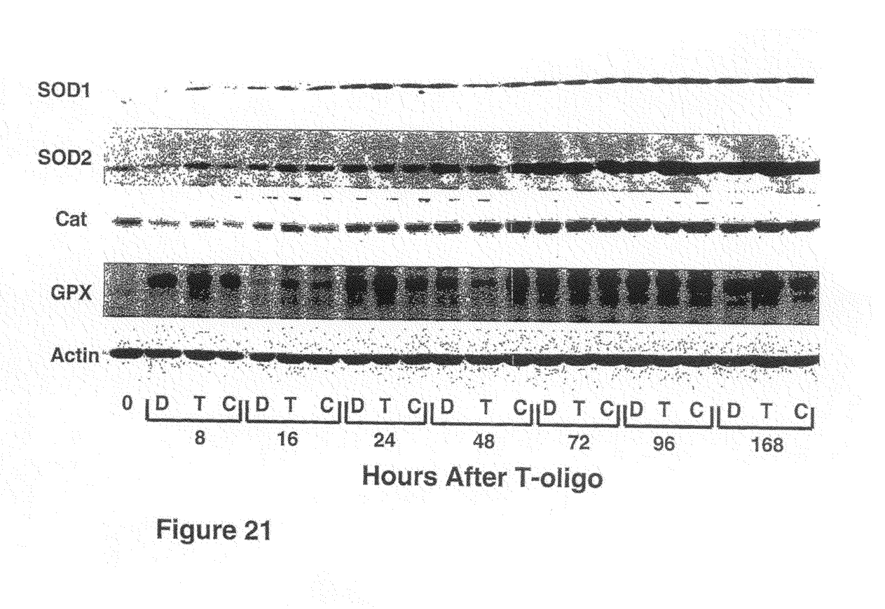

The levels of superoxide dismutase 1 (SOD1), superoxide dismutase 2 (SOD2), catalase (Cat) and glutathione peroxidase (GPX) were determined in newborn fibroblasts at different time points after treatment with 40 μM of T-oligo in comparison with cells treated either with 40 μM of control complementary oligo pCTAACCCTAAC (SEQ ID NO: 22) or diluent. In these measurements, newborn fibroblasts were plated in DMEM and 10% CS. Forty-eight hours after plating, cells were provided fresh medium supplemented with the T-oligo pGTTAGGGTTAG (40 μM) (T, SEQ ID NO: 1), with control complementary oligo pCTAACCCTAAC (40 μM) (C, SEQ ID NO: 22) or diluent. Total cellular proteins were harvested up to 168 hours after stimulation and processed for western blotting. The blot was sequentially reacted with antibodies against superoxide dismutase 1 (SOD1), superoxide dismutase 2 (SOD2), catalase (Cat) glutathione peroxidase (GPX) and actin as a loading control. As shown in FIG. 21, T-oligo induced the levels of SOD1, SOD2, and GPX within several hours after treatment.

The mechanism by which T-oligo affects fibroblasts involves activation of ATM²⁵⁵ and perhaps other PI3 kinases¹³, leading to activation of their downstream effector molecules, one of which is p53. Through these proteins, T-oligo induces a variety of DNA damage responses in fibroblasts including cell cycle arrest and senescence^{68,72}. Like T-oligo treatment, telomere maintenance and DNA damage response pathways involve induction and activation of p53, which can then stimulate NAD(P)H oxidases through p53-induced genes (PIGs) with redox activity¹⁶⁹. Of note, a majority of fibroblasts treated with pTT or 11mer-1 did not display S.A. β-gal activity after 72 hours of treatment. This leaves room to speculate that the increased levels of ROS are present and may even mediate other p53-related adaptive responses. Indeed, studies of adaptation to oxidative stress and radiation indicate that fibroblasts can develop adaptive resistance to noxious stimuli^{143,144,146}, and pretreating fibroblasts with T-oligos may increase their resistance to oxidative stress (FIGS. 20 and 21).

### T-Oligos and the Study of p53-Dependent NAD(P)H Oxidase Signaling

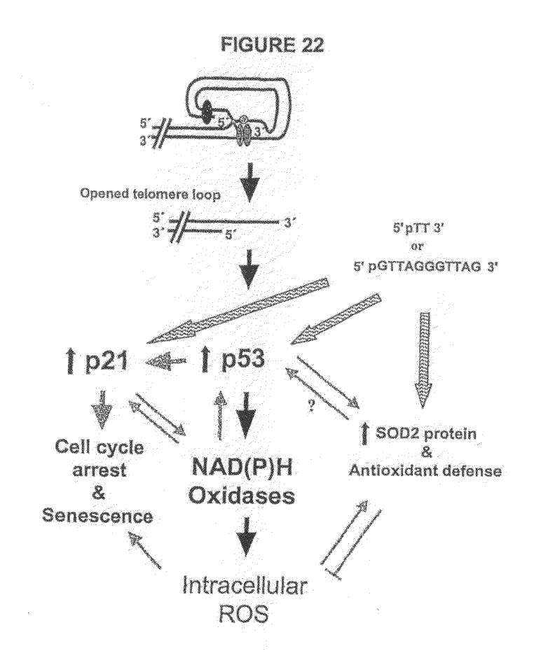

In summary, these results demonstrate the existence of p53-dependent redox responses to telomere homolog oligonucleotides. FIG. 22 summarizes the hypothesis by which T-oligos affect intracellular redox responses that may stimulate other protective responses. It is proposed that T-oligo treatment mimics the disruption of the telomere loop, which can occur with DNA damage. Telomere maintenance and DNA damage response pathways involve induction and activation of p53, which then stimulate NAD(P)H oxidases. The degree of p53 and ROS stimulation, balanced by antioxidant defense, are likely to determine the outcome of such stimulation, whether it is an adaptive and protective state or an irreversible endpoint leading to senescence or apoptosis. Much remains to be elucidated regarding redox responses to DNA damage, and T-oligo treatment in human dermal fibroblasts provides a novel model with which to explore the relationship between ROS, antioxidant defense and DNA damage responses. The existence of these antioxidant responses to T-oligos supports the existence of a coordinated eukaryotic SOS-like response to protect cells from further DNA damage. This model may also yield further insight into the relationship between telomere maintenance and function, antioxidant defense and ROS-stimulated signaling in the process of intrinsic aging or the development of age-related diseases.

### LIST OF ABBREVIATIONS

AOE—antioxidant enzyme

CAT—catalase

CS—calf serum

DCF—dichlorofluorescein [diacetate]

DMEM—Dulbecco's Modified Eagle's Medium

DMSO—dimethyl sulfoxide

DPI—diphenyliodonium chloride

FACS—fluorescence-activated cell sorter

FADH, FADH₂—flavin adenine dinucleotide, reduced form

FBS—fetal bovine serum

GPX—glutathione peroxidase

H₂O₂—hydrogen peroxide

HRP—horseradish peroxidase

NADH—nicotinamide adenine dinucleotide, reduced form

NADPH—nicotinamide adenine dinucleotide phosphate, reduced form

O₂.⁻—superoxide radical

NO.—nitric oxide radical

OH.—hydroxyl radical

p53DN—dominant negative p53

p53-ser15Phos—p53 phosphorylated on serine 15

ROS—reactive oxygen species

SA-β-gal—senescence-associated beta-galactosidase

SOD1—superoxide dismutase 1 (copper/zinc-dependent)

SOD2—superoxide dismutase 2 (manganese-dependent)

UV—ultraviolet

WT—wild-type

### REFERENCES

1. Hayflick, L. A brief history of the mortality and immortality of cultured cells. Keio J Med 47, 174-182 (1998).
2. Harman, D. J. Aging: a theory based on free radical and radiation chemistry. Gerontology 11, 298-300 (1956).
3. Halliwell, B. & Gutteridge, J. M. C. (eds.). Free radicals in biology and medicine, 784-802 (Oxford University Press, New York, 1999).
4. Droge, W. Free radicals in the physiological control of cell function. Physiol Rev 82, 47-95 (2002).
5. Takano, H. et al. Oxidative stress-induced signal transduction pathways in cardiac myocytes: involvement of ROS in heart diseases. Antioxid Redox Signal 5, 789-94 (2003).
6. Mates, J. M., Perez-Gomez, C. & Nunez de Castro, I. Antioxidant enzymes and human diseases. Clin Biochem 32, 595-603 (1999).
7. Bedogni, B. et al. Redox regulation of cAMP-responsive element-binding protein and induction of manganous superoxide dismutase in nerve growth factor-dependent cell survival. J Biol Chem 278, 16510-9 (2003).
8. Sarnbo, P. et al. Oxidative stress in scleroderma: maintenance of scleroderma fibroblast phenotype by the constitutive up-regulation of reactive oxygen species generation through the NADPH oxidase complex pathway. Arthritis Rheum 44, 2653-64 (2001).
9. Von Kobbe, C., May, A., Grandori, C. & Bohr, V. A. Werner syndrome cells escape hydrogen peroxide-induced cell proliferation arrest. Faseb J 18, 1970-2 (2004).
10. Macmillan-Crow, L. A. & Cruthirds, D. L. Invited review: manganese superoxide dismutase in disease. Free Radic Res 34, 325-36 (2001).
11. Davies, K. J. The broad spectrum of responses to oxidants in proliferating cells: a new paradigm for oxidative stress. IUBMB Life 48, 41-7 (1999).
12. von Zglinicki, T., Saretzki, G., Docke, W. & Lotze, C. Mild hyperoxia shortens telomeres and inhibits proliferation of fibroblasts: a model for senescence? Exp Cell Res 220, 186-93 (1995).
13. Eller, M. S., Li, G. Z., Firoozabadi, R., Puri, N. & Gilchrest, B. A. Induction of a p95/Nbs1-mediated S phase checkpoint by telomere 3′ overhang specific DNA. Faseb J 17, 152-62 (2003).
14. Hayflick, L. & Moorehead, P. S. The serial cultivation of human diploid cell strains. Exp Cell Res 25, 585-621 (1961).
15. Blackburn, E. H. Structure and function of telomeres. Nature 350, 569-73 (1991).
16. Chen, Q., Fischer, A., Reagan, J. D., Yan, L. J. & Ames, B. N. Oxidative DNA damage and senescence of human diploid fibroblast cells. Proc Natl Acad Sci USA 92, 4337-41 (1995).
17. Opresko, P. L., Fan, J., Danzy, S., Wilson, D. M., 3rd & Bohr, V. A. Oxidative damage in telomeric DNA disrupts recognition by TRF1 and TRF2. Nucleic Acids Res 33, 1230-9 (2005).
18. Muller, H. J. The remaking of chromosomes. The collecting net-Woods Hole 13, 181-195 (1938).
19. Blackburn, E. H. & J. G., G. A tandemly repeated sequence at the termini of the extrachromosomal ribosomal RNA genes in Tetrahymena. J Mol Biol 120, 33-53 (1978).
20. Henderson, E., Hardin, C. C., Walk, S. K., Tinoco, I., Jr. & Blackburn, E. H. Telomeric DNA oligonucleotides form novel intramolecular structures containing guanine-guanine base pairs. Cell 51, 899-908 (1987).
21. Sundquist, W. I. & Klug, A. Telomeric DNA dimerizes by formation of guanine tetrads between hairpin loops. Nature 342, 825-9 (1989).
22. Moyzis R K, B.J., Cram L S, Dani M, Deaven L L, Jones M D, Meyne J, Ratliff R L, Wu J R. A highly conserved repetitive DNA sequence, (TTAGGG)n, present at the telomeres of human chromosomes. Proc Natl Acad Sci USA 85, 6622-6626 (1988).
23. McElligott, R. & Wellinger, R. J. The terminal DNA structure of mammalian chromosomes. Embo J 16, 3705-14 (1997).
24. Griffith J D, C.L., Rosenfield S, Stansel R M, Bianchi A, Moss H, de Lange T. Mammalian telomeres end in a large duplex loop. Cell 97, 503-514 (1999).
25. Luderus,-M. E. et al. Structure, subnuclear distribution, and nuclear matrix association of the mammalian telomeric complex. J Cell Biol 135, 867-81 (1996).
26. Bianchi, A., Smith, S., Chong, L., Elias, P. & de Lange, T. TRF1 is a dimer and bends telomeric DNA. Embo J 16, 1785-94 (1997).
27. Broccoli, D., Smogorzewska, A., Chong, L. & de Lange, T. Human telomeres contain two distinct Myb-related proteins, TRF1 and TRF2. Nat Genet 17, 231-235 (1997).
28. Stansel, R. M., de Lange, T. & Griffith, J. D. T-loop assembly in vitro involves binding of TRF2 near the 3′ telomeric overhang. Embo J 20, 5532-40 (2001).
29. Baumann, P. & Cech, T. R. Pot1, the putative telomere end-binding protein in fission yeast and humans. Science 292, 1171-5 (2001).
30. Yang, Q., Zheng, Y. L. & Harris, C. C. POT1 and TRF2 cooperate to maintain telomeric integrity. Mol Cell Biol 25, 1070-80 (2005).
31. Lei, M., Podell, E. R. & Cech, T. R. Structure of human POT1 bound to telomeric single-stranded DNA provides a model for chromosome end-protection. Nat Struct Mol Biol 11, 1223-9 (2004).
32. Zhu, X. D., Kuster, B., Mann, M., Petrini, J. H. & de Lange, T. Cell-cycle-regulated association of RAD50/MRE11/NBS1 with TRF2 and human telomeres. Nat Genet 25, 347-52 (2000).
33. Chong, L. et al. A human telomeric protein. Science 270, 1663-1667 (1995).
34. Bilaud, T. et al. Telomeric localization of TRF2, a novel human telobox protein. Nat Genet 17, 236-239 (1997).
35. van Steensel, B., Smogorzewska, A. & de Lange, T. TRF2 protects human telomeres from end-to-end fusions. Cell 92, 401-13 (1998).
36. Chin, L. et al. p53 deficiency rescues the adverse effects of telomere loss and cooperates with telomere dysfunction to accelerate carcinogenesis. Cell 97, 527-538 (1999).
37. Olovnikov, A. M. A theory of marginotomy. The incomplete copying of template margin in enzymic synthesis of polynucleotides and biological significance of the phenomenon. J Theor Biol 41, 181-190 (1973).
38. Levy, M. Z., Allsopp, R. C., Futcher, A. B., Greider, C. W. & Harley, C. B. Telomere end-replication problem and cell aging. J Mol Biol 225, 951-60 (1992).
39. Greider C W, B.E. Identification of a specific telomere terminal transferase activity in Tetrahymena extracts. Cell 43, 405-413 (1985).
40. Nakamura T M, M.G., Chapman K B, Weinrich S L, Andrews W H, Lingner J, Rarely C B, Cech T R. Telomerase catalytic subunit homologs from fission yeast and human. Science 277, 955-957 (1997).
41. Feng J, F.W., Wang S S, Weinrich S L, Avilion A A, Chiu C P, Adams R R, Chang E, Allsopp R C, Yu J, Le S, West M D, Harley C B, Andrews W H, Greider C W, Villeponteau B. The RNA component of human telomerase. Science 269, 1236-1241 (1995).
42. Harrington, L. et al. A mammalian telomerase-associated protein. Science 275, 973-977 (1997).
43. Kim, N. et al. Specific association of human telomerase activity with immortal cells and cancer. Science 266, 2011-2015 (1994).
44. Masutomi, K. et al. Telomerase maintains telomere structure in normal human cells. Cell 114, 241-53 (2003).
45. Osanai, M., Tamaki, T., Yonekawa, M., Kawamura, A. & Sawada, N. Transient increase in telomerase activity of proliferating fibroblasts and endothelial cells in granulation tissue of the human skin. Wound Repair Regen 10, 59-66 (2002).
46. Harley C B, F.A., Greider C W. Telomeres shorten during ageing of human fibroblasts. Nature 345, 458-460 (1990).
47. Martens, U. M., Chavez, E. A., Poon, S. S., Schmoor, C. & Lansdorp, P. M. Accumulation of short telomeres in human fibroblasts prior to replicative senescence. Exp Cell Res 256, 291-9 (2000).
48. Bodnar, A. G. et al. Extension of life-span by introduction of telomerase into normal human cells. Science 279, 349-52 (1998).
49. Allsopp, R. C. et al. Telomere length predicts replicative capacity of human fibroblasts. Proc Natl Acad Sci USA 89, 10114-8 (1992).
50. Crabbe, L., Verdun, R. E., Haggblom, C. I. & Karlseder, J. Defective telomere lagging strand synthesis in cells lacking WRN helicase activity. Science 306, 1951-3 (2004).
51. Herrera, E. et al. Disease states associated with telomerase deficiency appear earlier in mice with short telomeres. EMBO J 18, 2950-2960 (1999).
52. Chiu, C. P. & Harley, C. B. Replicative senescence and cell immortality: the role of telomeres and telomerase. Proc Soc Exp Biol Med 214, 99-106 (1997).
53. Hastie, N. D. et al. Telomere reduction in human colorectal carcinoma and with ageing. Nature 346, 866-868 (1990).
54. de Lange, T. et al. Structure and variability of human chromosome ends. Mol Cell Biol 10, 518-527 (1990).
55. Morales C P, H.S., Ouellete M, Kaur K J, Yan Y, Wilson K S, White M A, Wright W E, Shay J W. Absence of cancer-associated changes in human fibroblasts immortalized with telomerase. Nature Genetics 21, 115-8 (1999).
56. Ouellette M M, L.M., Herbert B S, Johnson M, Holt S E, Liss H S, Shay J W, Wright W E. Subsenescent telomere lengths in fibroblasts immortalized by limiting amounts of telomerase. J Biol Chem 275, 10072-6 (2000).
57. Blasco, M. A. et al. Telomere shortening and tumor formation by mouse cells lacking telomerase RNA. Cell 91, 25-34 (1997).
58. Zhu J, W. H., Bishop M, Blackburn E H. Telomerase extends the lifespan of virus-transformed human cells without net telomere lengthening. Proc Natl Acad Sci USA 96, 3723-3728 (1999).
59. Kang, M. K., Wentong, G. & Park, N.-H. Replicative senescence of normal human oral keratinocytes is associated with the loss of telomerase activity without shortening of telomeres. Cell Growth & Diff 9, 85-95 (1998).
60. Sitte, N., Saretzki, G. & von Zglinicki, T. Accelerated telomere shortening in fibroblasts after extended periods of confluency. Free Radic Biol Med 24, 885-93 (1998).
61. Lanza R P, C.J., Blackwell C, Cristofalo V J, Francis M K, Baerlocker G M, Mak J, Schertzer M, Chavez E A, Sawyer N, Lansdorp P M, West M D. Extension of cell life-span and telomere length in animals cloned from senescent somatic cells. Science 288, 665-669 (2000).
62. Williams, N. Death of Dolly marks cloning milestone. Curr Biol 13, R209-10 (2003).
63. Hastie, N. D. et al. Telomere reduction in human colorectal carcinoma and with ageing. Nature 346, 866-8 (1990).
64. Bryan, T. M., Marusic, L., Bacchetti, S., Namba, M. & Reddel, R. R. The telomere lengthening mechanism in telomerase-negative immortal human cells does not involve the telomerase RNA subunit. Hum Mol Genet 6, 921-6 (1997).
65. Reddel, R. R., Bryan, T. M. & Murnane, J. P. Immortalized cells with no detectable telomerase activity. A review. Biochemistry (Mosc) 62, 1254-62 (1997).
66. Blackburn, E. H. Telomere states and cell fates. Nature 408, 53-6 (2000).
67. Karlseder, J., Smogorzewska, A. & de Lange, T. Senescence induced by altered telomere state, not telomere loss. Science 295, 2446-9 (2002).
68. Li, G. Z., Eller, M. S., Firoozabadi, R. & Gilchrest, B. A. Evidence that exposure of the telomere 3′ overhang sequence induces senescence. Proc Natl Acad Sci USA 100, 527-31 (2003).
69. van Steensel, B. & de Lange, T. Control of telomere length by the human telomeric protein TRF1. Nature 385, 740-3 (1997).
70. Smogorzewska, A. et al. Control of human telomere length by TRF1 and TRF2. Mol Cell Biol 20, 1659-68 (2000).
71. Karlseder, J., Broccoli, D., Dai, Y., Hardy, S. & de Lange, T. p53- and ATM-dependent apoptosis induced by telomeres lacking TRF2. Science 283, 1321-5 (1999).
72. Li, G. Z., Eller, M. S., Hanna, K. & Gilchrest, B. A. Signaling pathway requirements for induction of senescence by telomere homolog oligonucleotides. Exp Cell Res 301, 189-200 (2004).
73. Sies, H. (ed.) Oxidative Stress II: Oxidants and Antioxidants, (Academic Press, London, 1991).
74. Halliwell, B. & Gutteridge, J. M. C. (eds.). Free radicals in biology and medicine, 25 (Oxford University Press, New York, 1999).
75. Toussaint, O., Medrano, E. E. & von Zglinicki, T. Cellular and molecular mechanisms of stress-induced premature senescence (SIPS) of human diploid fibroblasts and melanocytes. Exp Gerontol 35, 927-45 (2000).
76. Staniek, K. & Nohl, H. Are mitochondria a permanent source of reactive oxygen species? Biochim Biophys Acta 1460, 268-75 (2000).
77. Farber, J. L., Kyle, M. E. & Coleman, J. B. Mechanisms of cell injury by activated oxygen species. Lab Invest 62, 670-9 (1990).
78. Halliwell B, G.J. (ed.) Free radicals in biology and medicine, 48-100 (Oxford University Press, New York, 1999).
79. Halliwell, B. & Gutteridge, J. M. C. (eds.). Free radicals in biology and medicine, 278 (Oxford University Press, New York, 1999).
80. Halliwell B, G.J. (ed.) Free radicals in biology and medicine, 268 (Oxford University Press, New York, 1999).
81. Beckman, K. B. & Ames, B. N. The free radical theory of aging matures. Physiol Rev 78, 547-81 (1998).
82. Esposito, L. A., Melov, S., Panov, A., Cottrell, B. A. & Wallace, D. C. Mitochondrial disease in mouse results in increased oxidative stress. Proc Natl Acad Sci U S A 96, 4820-5 (1999).
83. Anson, R. M., Hudson, E. & Bohr, V. A. Mitochondrial endogenous oxidative damage has been overestimated. Faseb J 14, 355-60 (2000).
84. Sohal, R. S. & Weindruch, R. Oxidative stress, caloric restriction, and aging. Science 273, 59-63 (1996).
85. Miguel, J., Economos, A. C., Fleming, J. & Johnson, J. E., Jr. Mitochondrial role in cell aging. Exp Gerontol 15, 575-91 (1980).
86. Harman, D. The biologic clock: the mitochondria? J Am Geriatr Soc 20, 145-7 (1972).
87. Gross, A., McDonnell, J. M. & Korsmeyer, S. J. BCL-2 family members and the mitochondria in apoptosis. Genes & Development 13, 1899-1911 (1999).
88. Black, H. S. et al. Photocarcinogenesis: an overview. J Photochem Photobiol B 40, 29-47 (1997).
89. Hochstein, P. & Atallah, A. S. The nature of oxidants and antioxidant systems in the inhibition of mutation and cancer. Mutat Res 202, 363-75 (1988).
90. Bohr, V. A. & Dianov, G. L. Oxidative DNA damage processing in nuclear and mitochondrial DNA. Biochimie 81, 155-60 (1999).
91. Petersen, S., Saretzki, G. & von Zglinicki, T. Preferential accumulation of single-stranded regions in telomeres of human fibroblasts. Exp Cell Res 239, 152-60 (1998).
92. Halliwell B, G.J. (ed.) Free radicals in biology and medicine, 271 (Oxford University Press, New York, 1999).
93. Epe, B., Pflaum, M. & Boiteux, S. DNA damage induced by photosensitizers in cellular and cell-free systems. Mutat Res 299, 135-45 (1993).
94. Oikawa, S. & Kawanishi, S. Site-specific DNA damage at GGG sequence by oxidative stress may accelerate telomere shortening. FEBS Lett 453, 365-8 (1999).
95. Saretzki, G., Sitte, N., Merkel, U., Wurm, R. E. & von Zglinicki, T. Telomere shortening triggers a p53-dependent cell cycle arrest via accumulation of G-rich single stranded DNA fragments. Oncogene 18, 5148-58 (1999).
96. Furumoto, K., Inoue, E., Nagao, N., Hiyama, E. & Miwa, N. Age-dependent telomere shortening is slowed down by enrichment of intracellular vitamin C via suppression of oxidative stress. Life Sci 63, 935-48 (1998).
97. Wong, K. K. et al. Telomere dysfunction impairs DNA repair and enhances sensitivity to ionizing radiation. Nat Genet 26, 85-8 (2000).
98. Ramirez, R. et al. Massive telomere loss is an early event of DNA damage-induced apoptosis. J Biol Chem 278, 836-42 (2003).
99. von Zglinicki, T. Role of oxidative stress in telomere length regulation and replicative senescence. Ann NY Acad Sci 908, 99-110 (2000).
100. Proctor, C. J. & Kirkwood, T. B. Modelling telomere shortening and the role of oxidative stress. Mech Ageing Dev 123, 351-63 (2002).
101. Halliwell, B. & Gutteridge, J. M. C. (eds.). Free radicals in biology and medicine, 106-107 (Oxford University Press, New York, 1999).
102. Halliwell, B. & Gutteridge, J. M. C. (eds.). Free radicals in biology and medicine, 823 (Oxford University Press, New York, 1999).
103. Sgambato, A. et al. Resveratrol, a natural phenolic compound, inhibits cell proliferation and prevents oxidative DNA damage. Mutat Res 496, 171-80 (2001).
104. Leccia, M. T., Yaar, M., Allen, N., Gleason, M. & Gilchrest, B. A. Solar simulated irradiation modulates gene expression and activity of antioxidant enzymes in cultured human dermal fibroblasts. Exp Dermatol 10, 272-9 (2001).
105. Halliwell, B. & Gutteridge, J. M. C. (eds.). Free radicals in biology and medicine, 107-112 (Oxford University Press, New York, 1999).
106. Slot, J. W., Geuze, H. J., Freeman, B. A. & Crapo, J. D. Intracellular localization of the copper-zinc and manganese superoxide dismutases in rat liver parenchymal cells. Lab Invest 55, 363-71 (1986).
107. Halliwell, B. & Gutteridge, J. M. C. (eds.). Free radicals in biology and medicine, 112-113 (Oxford University Press, New York, 1999).
108. Halliwell, B. & Gutteridge, J. M. C. (eds.). Free radicals in biology and medicine, 134-139 (Oxford University Press, New York, 1999).
109. Halliwell B, G.J. (ed.) Free radicals in biology and medicine, 107-150 (Oxford University Press, New York, 1999).
110. Bravard, A., Petridis, F. & Luccioni, C. Modulation of antioxidant enzymes p21WAF1 and p53 expression during proliferation and differentiation of human melanoma cell lines. Free Radic Biol Med 26, 1027-33 (1999).
111. Serra, V., von Zglinicki, T., Lorenz, M. & Saretzld, G. Extracellular superoxide dismutase is a major antioxidant in human fibroblasts and slows telomere shortening. J Biol Chem 278, 6824-30 (2003).
112. Keyse, S. M. & Tyrrell, R. M. Heme oxygenase is the major 32-kDa stress protein induced in human skin fibroblasts by UVA radiation, hydrogen peroxide, and sodium arsenite. Proc Natl Acad Sci USA 86, 99-103 (1989).
113. Southorn, P. A. & Powis, G. Free radicals in medicine. I. Chemical nature and biologic reactions. Mayo Clin Proc 63, 381-9 (1988).
114. Szabo, C. Multiple pathways of peroxynitrite cytotoxicity. Toxicol Lett 140-141, 105-12 (2003).
115. Halliwell B, G.J. (ed.) Free radicals in biology and medicine, 177-181 (Oxford University Press, New York, 1999).
116. Xing, J., Yu, Y. & Rando, T. A. The modulation of cellular susceptibility to oxidative stress: protective and destructive actions of Cu,Zn-superoxide dismutase. Neurobiol Dis 10, 234-46 (2002).
117. Halliwell B, G.J. (ed.) Free radicals in biology and medicine, 796 (Oxford University Press, New York, 1999).
118. Allen, R. G. et al. Expression and regulation of superoxide dismutase activity in human skin fibroblasts from donors of different ages. J Cell Physiol 165, 576-87 (1995).
119. Keogh, B. P. et al. Expression of hydrogen peroxide and glutathione metabolizing enzymes in human skin fibroblasts derived from donors of different ages. J Cell Physiol 167, 512-22 (1996).
120. Ji, L. L. Exercise-induced modulation of antioxidant defense. Ann NY Acad Sci 959, 82-92 (2002).
121. Stadtman, E. R., Starke-Reed, P. E., Oliver, C. N., Carney, J. M. & Floyd, R. A. Protein modification in aging. Exs 62, 64-72 (1992).
122. Stadtman, E. R. Protein oxidation in aging and age-related diseases. Ann NY Acad Sci 928, 22-38 (2001).
123. Kasapoglu, M. & Ozben, T. Alterations of antioxidant enzymes and oxidative stress markers in aging. Exp Gerontol 36, 209-20 (2001).
124. Schmidt, A. M., Yan, S. D., Yan, S. F. & Stem, D. M. The biology of the receptor for advanced glycation end products and its ligands. Biochim Biophys Acta 1498, 99-111 (2000).
125. Seidel, W. & Pischetsrieder, M. Immunochemical detection of N2-[1-(1-carboxy)ethyl]guanosine, an advanced glycation end product formed by the reaction of DNA and reducing sugars or L-ascorbic acid in vitro. Biochim Biophys Acta 1425, 478-84 (1998).
126. Verbeke, P., Clark, B. F. & Rattan, S. I. Modulating cellular aging in vitro: hormetic effects of repeated mild heat stress on protein oxidation and glycation. Exp Gerontol 35, 787-94 (2000).
127. Taylor, R. P. & Starnes, J. W. Age, cell signalling and cardioprotection. Acta Physiol Scand 178, 107-16 (2003).
128. Gaitanaki, C., Konstantina, S., Chrysa, S. & Beis, I. Oxidative stress stimulates multiple MAPK signalling pathways and phosphorylation of the small HSP27 in the perfused amphibian heart. J Exp Biol 206, 2759-69 (2003).
129. Reenstra, W. R., Yaar, M. & Gilchrest, B. A. Effect of donor age on epidermal growth factor processing in man. Exp Cell Res 209, 118-22 (1993).
130. Reenstra, W. R., Yaar, M. & Gilchrest, B. A. Aging affects epidermal growth factor receptor phosphorylation and traffic kinetics. Exp Cell Res 227, 252-5 (1996).
131. Halliwell B, G.J. (ed.) Free radicals in biology and medicine, 792, 798-799 (Oxford University Press, New York, 1999).
132. Huang, T. T. et al. Superoxide-mediated cytotoxicity in superoxide dismutase-deficient fetal fibroblasts. Arch Biochem Biophys 344, 424-32 (1997).
133. Li, Y. et al. Dilated cardiomyopathy and neonatal lethality in mutant mice lacking manganese superoxide dismutase. Nat Genet 11, 376-81 (1995).
134. Melov, S. et al. Extension of life-span with superoxide dismutase/catalase mimetics. Science 289, 1567-9 (2000).
135. Merry, B. J. Calorie restriction and age-related oxidative stress. Ann NY Acad Sci 908, 180-98 (2000).
136. Lin, S. J. et al. Calorie restriction extends Saccharomyces cerevisiae lifespan by increasing respiration. Nature 418, 344-8 (2002).
137. Guarente, L. & Picard, F. Calorie restriction—the SIR2 connection. Cell 120, 473-82 (2005).
138. Brunet, A. et al. Stress-dependent regulation of FOXO transcription factors by the SIRT1 deacetylase. Science 303, 2011-5 (2004).
139. Daitoku, H. et al. Silent information regulator 2 potentiates Foxo1-mediated transcription through its deacetylase activity. Proc Natl Acad Sci USA 101, 10042-7 (2004).
140. Migliaccio, E. et al. The p66shc adaptor protein controls oxidative stress response and life span in mammals. Nature 402, 309-13 (1999).
141. Trinei, M. et al. A p53-p66Shc signalling pathway controls intracellular redox status, levels of oxidation-damaged DNA and oxidative stress-induced apoptosis. Oncogene 21, 3872-8 (2002).
142. Eller, M. S., Maeda, T., Magnoni, C., Atwal, D. & Gilchrest, B. A. Enhancement of DNA repair in human skin cells by thymidine dinucleotides: evidence for a p53-mediated mammalian SOS response. Proc Natl Acad Sci USA 94, 12627-32 (1997).
143. Wiese, A. G., Pacifici, R. E. & Davies, K. J. Transient adaptation of oxidative stress in mammalian cells. Arch Biochem Biophys 318, 231-40 (1995).
144. Poswig, A. et al. Adaptive antioxidant response of manganese-superoxide dismutase following repetitive UVA irradiation. J Invest Dermatol 112, 13-8 (1999).
145. Bravard, A., Luccioni, C., Moustacchi, E. & Rigaud, O. Contribution of antioxidant enzymes to the adaptive response to ionizing radiation of human lymphoblasts. Int J Radiat Biol 75, 639-45 (1999).
146. Rigaud, O. & Moustacchi, E. Radioadaptation for gene mutation and the possible molecular mechanisms of the adaptive response. Mutat Res 358, 127-34 (1996).
147. Gros, L., Saparbaev, M. K. & Laval, J. Enzymology of the repair of free radicals-induced DNA damage. Oncogene 21, 8905-25 (2002).
148. Gilchrest, B. A. & Eller, M. S. DNA photodamage stimulates melanogenesis and other photoprotective responses. J Investig Dermatol Symp Proc 4, 35-40 (1999).
149. Ramana, C. V., Boldogh, I., Izumi, T. & Mitra, S. Activation of apurinic/apyrimidinic endonuclease in human cells by reactive oxygen species and its correlation with their adaptive response to genotoxicity of free radicals. Proc Natl Acad Sci USA 95, 5061-6 (1998).
150. Eller, M. S., Yaar, M. & Gilchrest, B. A. DNA damage and melanogenesis. Nature 372, 413-4 (1994).
151. Eller, M. S., Ostrom, K. & Gilchrest, B. A. DNA damage enhances melanogenesis. Proc Natl Acad Sci USA 93, 1087-92 (1996).
152. Iyer, R. & Lehnert, B. E. Alpha-particle-induced increases in the radioresistance of normal human bystander cells. Radiat Res 157, 3-7 (2002).
153. Azzam, E. I., De Toledo, S. M., Spitz, D. R. & Little, J. B. Oxidative metabolism modulates signal transduction and micronucleus formation in bystander cells from alpha-particle-irradiated normal human fibroblast cultures. Cancer Res 62, 5436-42 (2002).
154. Hardmeier, R., Hoeger, H., Fang-Kircher, S., Khoschsorur, A. & Lubec, G. Transcription and activity of antioxidant enzymes after ionizing irradiation in radiation-resistant and radiation-sensitive mice. Proc Natl Acad Sci USA 94, 7572-6 (1997).
155. Rogers, R. J., Chesrown, S. E., Kuo, S., Monnier, J. M. & Nick, H. S. Cytokine-inducible enhancer with promoter activity in both the rat and human manganese-superoxide dismutase genes. Biochem J 347 Pt 1, 233-42 (2000).
156. Isoherranen, K. et al. Regulation of copper/zinc and manganese superoxide dismutase by UVB irradiation, oxidative stress and cytokines. J Photochem Photobiol B 40, 288-93 (1997).
157. Stralin, P. & Marklund, S. L. Effects of oxidative stress on expression of extracellular superoxide dismutase, CuZn-superoxide dismutase and Mn-superoxide dismutase in human dermal fibroblasts. Biochem J298 (Pt 2), 347-52 (1994).
158. Shull, S. et al. Differential regulation of antioxidant enzymes in response to oxidants. J Biol Chem 266, 24398-403 (1991).
159. Offer, H. et al. Direct involvement of p53 in the base excision repair pathway of the DNA repair machinery. FEBS Lett 450, 197-204 (1999).
160. Offer, H. et al. Structural and functional involvement of p53 in BER in vitro and in vivo. Oncogene 20, 581-9 (2001).
161. Smith, M. L. & Seo, Y. R. p53 regulation of DNA excision repair pathways. Mutagenesis 17, 149-56 (2002).
162. Offer, H. et al. p53 modulates base excision repair activity in a cell cycle-specific manner after genotoxic stress. Cancer Res 61, 88-96 (2001).
163. Jayaraman, L. et al. Identification of redox/repair protein Ref-1 as a potent activator of p53. Genes Dev 11, 558-70 (1997).
164. Gaiddon, C., Moorthy, N. C. & Prives, C. Ref-1 regulates the transactivation and pro-apoptotic functions of p53 in vivo. Embo J 18, 5609-21 (1999).
165. Agarwal, M. L., Taylor, W. R., Chernov, M. V., Chemova, O. B. & Stark, G. R. The p53 network. J Biol Chem 273, 1-4 (1998).
166. Drane, P., Bravard, A., Bouvard, V. & May, E. Reciprocal down-regulation of p53 and SOD2 gene expression-implication in p53 mediated apoptosis. Oncogene 20, 430-9 (2001).
167. Cho, G., Kang, S., Seo, S. J., Kim, Y. & Jung, G. The transcriptional repression of the human Cu/Zn superoxide dismutase(sod1) gene by the anticancer drug, mitomycin C(MMC). Biochem Mol Biol Int 42, 949-56 (1997).
168. Tan, M. et al. Transcriptional activation of the human glutathione peroxidase promoter by p53. J Biol Chem 274, 12061-6 (1999).
169. Polyak, K., Xia, Y., Zweier, J. L., Kinzler, K. W. & Vogelstein, B. A model for p53-induced apoptosis. Nature 389, 300-5 (1997).
170. Macip, S. et al. Inhibition of p21-mediated ROS accumulation can rescue p21-induced senescence. Embo J21, 2180-8 (2002).
171. Johnson, T. M., Yu, Z. X., Ferrans, V. J., Lowenstein, R. A. & Finkel, T. Reactive oxygen species are downstream mediators of p53-dependent apoptosis. Proc Nad Acad Sci USA 93, 11848-52 (1996).
172. Goukassian, D. A., Eller, M. S., Yaar, M. & Gilchrest, B. A. Thymidine dinucleotide mimics the effect of solar simulated irradiation on p53 and p53-regulated proteins. J Invest Dermatol 112, 25-31 (1999).
173. Khlgatian, M. K. et al. Tyrosinase gene expression is regulated by p53. J Invest Dermatol 118, 126-32 (2002).
174. Goukassian, D. A. et al. Topical DNA oligonucleotide therapy reduces UV-induced mutations and photocarcinogenesis in hairless mice. Proc Natl Acad Sci USA 101, 3933-8 (2004).
175. Hadshiew, I. M., Eller, M. S., Gasparro, F. P. & Gilchrest, B. A. Stimulation of melanogenesis by DNA oligonucleotides: effect of size, sequence and 5′ phosphorylation. J Dermatol Sci 25, 127-38 (2001).
176. Puri, N. et al. Telomere-based DNA damage responses: a new approach to melanoma. Faseb J 18, 1373-81 (2004).
177. Cruz, P. D., Jr. et al. Thymidine dinucleotides inhibit contact hypersensitivity and activate the gene for tumor necrosis factor alpha1. J Invest Dermatol 114, 253-8 (2000).
178. Anderson, R. R. & Parrish, J. A. The optics of human skin. J Invest Dermatol 77, 13-9 (1981).
179. Kvam, E. & Tyrrell, R. M. Induction of oxidative DNA base damage in human skin cells by UV and near visible radiation. Carcinogenesis 18, 2379-84 (1997).
180. Huet, O., Petit, J. M., Ratinaud, M. H. & Julien, R. NADH-dependent dehydrogenase activity estimation by flow cytometric analysis of 3-(4,5-dimethylthiazolyl-2-yl)-2,5-diphenyltetrazolium bromide (MTT) reduction. Cytometry 13, 532-9 (1992).
181. Busciglio, J. et al. Altered metabolism of the amyloid beta precursor protein is associated with mitochondrial dysfunction in Down's syndrome. Neuron 33, 677-88 (2002).
182. Church, S. L. Manganese superoxide dismutase: nucleotide and deduced amino acid sequence of a cDNA encoding a new human transcript. Biochim Biophys Acta 1087, 250-2 (1990).
183. Halliwell, B. & Gutteridge, J. M. C. (eds.). Free radicals in biology and medicine, (Oxford University Press, New York, 1999).
184. Sugawara, T. et al. Overexpression of copper/zinc superoxide dismutase in transgenic rats protects vulnerable neurons against ischemic damage by blocking the mitochondrial pathway of caspase activation. J Neurosci 22, 209-17 (2002).
185. Halliwell, B. & Gutteridge, J. M. C. (eds.). Free radicals in biology and medicine, 130-134 (Oxford University Press, New York, 1999).
186. Van Remmen, H. et al. Life-long reduction in MnSOD activity results in increased DNA damage and higher incidence of cancer but does not accelerate aging. Physiol Genomics 16, 29-37 (2003).
187. van den Burg, B. & Eijsink, V. G. Selection of mutations for increased protein stability. Curr Opin Biotechnol 13, 333-7 (2002).
188. Akashi, M. et al. Irradiation increases manganese superoxide dismutase mRNA levels in human fibroblasts. Possible mechanisms for its accumulation. J Biol Chem 270, 15864-9 (1995).
189. Yamashita, N. et al. Exercise provides direct biphasic cardioprotection via manganese superoxide dismutase activation. J Exp Med 189, 1699-706 (1999).
190. O'Brien, P. J. et al. Repeated acetaminophen dosing in rats: adaptation of hepatic antioxidant system. Hum Exp Toxicol 19, 277-83 (2000).
191. Oberley, T. D., Schultz, J. L. & Oberley, L. W. In vitro modulation of antioxidant enzyme levels in normal hamster kidney and estrogen-induced hamster kidney tumor. Free Radic Biol Med 16, 741-51 (1994).
192. Bowler, C. et al. A plant manganese superoxide dismutase is efficiently imported and correctly processed by yeast mitochondria. Proc Natl Acad Sci USA 86, 3237-41 (1989).
193. Bowler, C., Alliotte, T., De Loose, M., Van Montagu, M. & Inze, D. The induction of manganese superoxide dismutase in response to stress in Nicotiana plumbaginifolia. Embo J 8, 31-8 (1989).
194. Shackelford, R. E., Kaufmann, W. K. & Paules, R. S. Oxidative stress and cell cycle checkpoint function. Free Radic Biol Med 28, 1387-404 (2000).
195. Shackelford, R. E. et al. The Ataxia telangiectasia gene product is required for oxidative stress-induced G1 and G2 checkpoint function in human fibroblasts. J Biol Chem 276, 21951-9 (2001).
196. Cadenas, E. Mitochondrial free radical production and cell signaling. Mol Aspects Med 25, 17-26 (2004).
197. Ranganathan, A. C. et al. Manganese superoxide dismutase signals matrix metalloproteinase expression via H2O2-dependent ERK1/2 activation. J Biol Chem 276, 14264-70 (2001).
198. Melendez, J. A. & Davies, K. J. Manganese superoxide dismutase modulates interleukin-1alpha levels in HT-1080 fibrosarcoma cells. J Biol Chem 271, 18898-903 (1996).
199. Balaban, R. S., Nemoto, S. & Finkel, T. Mitochondria, oxidants, and aging. Cell 120, 483-95 (2005).
200. Berridge, M. V. & Tan, A. S. Characterization of the cellular reduction of 3-(4,5-dimethylthiazol-2-yl)-2,5-diphenyltetrazolium bromide (MTT): subcellular localization, substrate dependence, and involvement of mitochondrial electron transport in MTT reduction. Arch Biochem Biophys 303, 474-82 (1993).
201. Thannickal, V. J. & Fanburg, B. L. Reactive oxygen species in cell signaling. Am J Physiol Lung Cell Mol Physiol 279, L1005-28 (2000).
202. Gilchrest, B. A. Using DNA damage responses to prevent and treat skin cancers. J Dermatol 31, 862-77 (2004).
203. Meplan, C., Richard, M. J. & Hainaut, P. Redox signalling and transition metals in the control of the p53 pathway. Biochem Pharmacol 59, 25-33 (2000).
204. Forman, H. J. & Torres, M. Reactive oxygen species and cell signaling: respiratory burst in macrophage signaling. Am J Respir Crit Care Med 166, S4-8 (2002).
205. Denu, J. M. & Tanner, K. G. Specific and reversible inactivation of protein tyrosine phosphatases by hydrogen peroxide: evidence for a sulfenic acid intermediate and implications for redox regulation. Biochemistry 37, 5633-42 (1998).
206. Halliwell, B. & Gutteridge, J. M. C. (eds.). Free radicals in biology and medicine, 326-330 (Oxford University Press, New York, 1999).
207. Cho, H. J. et al. Oncogenic H-Ras enhances DNA repair through the Ras/phosphatidylinositol 3-kinase/Rac1 pathway in NIH3T3 cells. Evidence for association with reactive oxygen species. J Biol Chem 277, 19358-66 (2002).
208. Thannickal, V. J. The paradox of reactive oxygen species: injury, signaling, or both? Am J Physiol Lung Cell Mol Physiol 284, L24-5 (2003).
209. Meier, B., Cross, A. R., Hancock, J. T., Kaup, F. J. & Jones, O. T. Identification of a superoxide-generating NADPH oxidase system in human fibroblasts. Biochem J 275 (Pt 1), 241-5 (1991).
210. Meier, B., Jesaitis, A. J., Emmendorffer, A., Roesler, J. & Quinn, M. T. The cytochrome b-558 molecules involved in the fibroblast and polymorphonuclear leukocyte superoxide-generating NADPH oxidase systems are structurally and genetically distinct. Biochem J 289 (Pt 2), 481-6 (1993).
211. Ruch, W., Cooper, P. H. & Baggiolini, M. Assay of H2O2 production by macrophages and neutrophils with homovanillic acid and horse-radish peroxidase. J Immunol Methods 63, 347-57 (1983).
212. Arbault, S. et al. Activation of the NADPH oxidase in human fibroblasts by mechanical intrusion of a single cell with an ultramicroelectrode. Carcinogenesis 18, 569-74 (1997).
213. Thannickal, V. J. & Fanburg, B. L. Activation of an H2O2-generating NADH oxidase in human lung fibroblasts by transforming growth factor beta 1. J Biol Chem 270, 30334-8 (1995).
214. Meier, B. et al. Human fibroblasts release reactive oxygen species in response to interleukin-1 or tumour necrosis factor-alpha. Biochem J263, 539-45 (1989).
215. Han, D., Williams, E. & Cadenas, E. Mitochondrial respiratory chain-dependent generation of superoxide anion and its release into the intermembrane space. Biochem J 353, 411-6 (2001).
216. Turrens, J. F. Mitochondrial formation of reactive oxygen species. J Physiol 552, 335-44 (2003).
217. Taylor, E. R. et al. Reversible glutathionylation of complex I increases mitochondrial superoxide formation. J Biol Chem (2003).
218. Casteilla, L., Rigoulet, M. & Penicaud, L. Mitochondrial ROS metabolism: modulation by uncoupling proteins. IUBMB Life 52, 181-8 (2001).
219. Hartman, P., Ponder, R., Lo, H. H. & Ishii, N. Mitochondrial oxidative stress can lead to nuclear hypermutability. Mech Ageing Dev 125, 417-20 (2004).
220. Helt, C. E., Rancourt, R. C., Staversky, R. J. & O'Reilly, M. A. p53-dependent induction of p21(Cip1/WAF1/Sdi1) protects against oxygen-induced toxicity. Toxicol Sci 63, 214-22 (2001).
221. Dhenaut, A., Boiteux, S. & Radicella, J. P. Characterization of the hOGG1 promoter and its expression during the cell cycle. Mutat Res 461, 109-18 (2000).
222. Imai, K. et al. Genomic structure and sequence of a human homologue (NTHL1/NTH1) of Escherichia coli endonuclease III with those of the adjacent parts of TSC2 and SLC9A3R2 genes. Gene 222, 287-95 (1998).
223. Benhar, M., Dalyot, I., Engelberg, D. & Levitzki, A. Enhanced ROS production in oncogenically transformed cells potentiates c-Jun N-terminal kinase and p38 mitogen-activated protein kinase activation and sensitization to genotoxic stress. Mol Cell Biol 21, 6913-26 (2001).
224. Seimiya, H. et al. Hypoxia up-regulates telomerase activity via mitogen-activated protein kinase signaling in human solid tumor cells. Biochem Biophys Res Commun 260, 365-70 (1999).
225. Abe, J. & Berk, B. C. Fyn and JAK2 mediate Ras activation by reactive oxygen species. J Biol Chem 274, 21003-10 (1999).
226. Vialas, C., Pratviel, G. & Meunier, B. Oxidative damage generated by an oxo-metalloporphyrin onto the human telomeric sequence. Biochemistry 39, 9514-22 (2000).
227. Maeda, T., Eller, M. S., Hedayati, M., Grossman, L. & Gilchrest, B. A. Enhanced repair of benzo(a)pyrene-induced DNA damage in human cells treated with thymidine dinucleotides. Mutat Res 433, 137-45 (1999).
228. Eller, M. S., Puri, N., Hadshiew, I. M., Venna, S. S. & Gilchrest, B. A. Induction of apoptosis by telomere 3′ overhang-specific DNA. Exp Cell Res 276, 185-93 (2002).
229. Milyaysky, M. et al. Activation of p53 protein by telomeric (TTAGGG)n repeats. Nucleic Acids Res 29, 5207-15 (2001).
230. Eller, M. S. et al. Single-stranded telomeric DNA induces DNA damage responses. in Annual Meeting of the Society for Investigative Dermatology Vol. 114 756 (2000).
231. Curiel-Lewandrowski, C. et al. Inhibition of the elicitation phase of contact hypersensitivity by thymidine dinucleotides is in part mediated by increased expression of interleukin-10 in human keratinocytes. Exp Dermatol 12, 145-52 (2003).
232. Yaar, M.e.a., manuscript in preparation.
233. Li, G.Z.e.a.m.i.p.
234. Blackvall, H.e.a., manuscript in preparation.
235. Eller, M.S.e.a., manuscript in preparation.
236. Rheinwald, J. G. et al. A two-stage, p16(INK4A)- and p53-dependent keratinocyte senescence mechanism that limits replicative potential independent of telomere status. Mol Cell Biol 22, 5157-72 (2002). 237. Holland, P. C., Clark, M. G., Bloxham, D. P. & Lardy, H. A. Mechanism of action of the hypoglycemic agent diphenyleneiodonium. J Biol Chem 248, 6050-6 (1973).
238. Majander, A., Finel, M. & Wikstrom, M. Diphenyleneiodonium inhibits reduction of iron-sulfur clusters in the mitochondrial NADH-ubiquinone oxidoreductase (Complex I). J Biol Chem 269, 21037-42 (1994).
239. Chakraborty, S. & Massey, V. Reaction of reduced flavins and flavoproteins with diphenyliodonium chloride. J Biol Chem 277, 41507-16 (2002).
240. Dimri, G. P. et al. A biomarker that identifies senescent human cells in culture and in aging skin in vivo. Proc Natl Acad Sci USA 92, 9363-7 (1995).
241. Lee, A. C. et al. Ras proteins induce senescence by altering the intracellular levels of reactive oxygen species. J Biol Chem 274, 7936-40 (1999).
242. Tsuchiya, M., Suematsu, M. & Suzuki, H. In vivo visualization of oxygen radical-dependent photoemission. Methods Enzymol 233, 128-40 (1994).
243. Liu, G. & Chen, X. The ferredoxin reductase gene is regulated by the p53 family and sensitizes cells to oxidative stress-induced apoptosis. Oncogene 21, 7195-204 (2002).
244. Loke, S. L. et al. Characterization of oligonucleotide transport into living cells. Proc Natl Acad Sci USA 86, 3474-8 (1989).
245. Eller, M.e.a., manuscript in preparation:
246. Huffer, E., Unterluggauer, H., Uberall, F., Schramek, H. & Jansen-Durr, P. Replicative senescence of human fibroblasts: the role of Ras-dependent signaling and oxidative stress. Exp Gerontol 37, 1165-74 (2002).
247. Irani, K. et al. Mitogenic signaling mediated by oxidants in Ras-transformed fibroblasts. Science 275, 1649-52 (1997).
248. Irani, K. & Goldschmidt-Clermont, P. J. Ras, superoxide and signal transduction. Biochem Pharmacol 55, 1339-46 (1998).
249. Cross, A. R. & Jones, O. T. The effect of the inhibitor diphenylene iodonium on the superoxide-generating system of neutrophils. Specific labelling of a component polypeptide of the oxidase. Biochem J237, 111-6 (1986).
250. Brar, S. S. et al. An NAD(P)H oxidase regulates growth and transcription in melanoma cells. Am J Physiol Cell Physiol 282, C1212-24 (2002).
251. Hockberger, P. E. et al. Activation of flavin-containing oxidases underlies light-induced production of H2O2 in mammalian cells. Proc Natl Acad Sci U S A 96, 6255-60 (1999).
252. Boveris, A., Alvarez, S., Bustamante, J. & Valdez, L. Measurement of superoxide radical and hydrogen peroxide production in isolated cells and subcellular organelles. Methods Enzymol 349, 280-7 (2002).
253. Werrlein, R. J. & Glinos, A. D. Oxygen microenvironment and respiratory oscillations in cultured mammalian cells. Nature 251, 317-9 (1974).
254. Tokuda, Y., Crane, S., Yamaguchi, Y., Zhou, L. & Falanga, V. The levels and kinetics of oxygen tension detectable at the surface of human dermal fibroblast cultures. J Cell Physiol 182, 414-20 (2000).
255. Eller, M. S., Liao, X., Faravash, Y., and Gilchrest, B. A. A role for WRN and DNA-PK in telomere-based DNA damage responses including senescence. (2005).

We claim:

1. A method of treating an oxidative stress disorder in a mammal comprising administering to the mammal a pharmaceutical composition that comprises a telomere homolog oligonucleotide.

2. The method of claim 1 wherein the oligonucleotide has at least 33% sequence identity to (TTAGGG)ₙ, wherein n is a number from 1 to 333.

3. The method of claim 2 wherein the sequence identity is at least 50%.

4. The method of claim 1 wherein the oligonucleotide is selected from the group consisting of GAGTATGAG (SEQ ID NO: 5), GTTAGGGTTAG (SEQ ID NO: 6), GGGTTAGGGTT (SEQ ID NO: 7), TAGATGTGGTG (SEQ ID NO: 8) and TT, said oligonucleotide optionally comprising a 5′-phosphate.

5. The method of claim 1 wherein the mammal is a human.

6. The method of claim 1 wherein the oxidative stress disorder is selected from the group consisting of retinal degeneration, Alzheimer's disease, aging, skin photoaging, cardiovascular disease, hypertension, hypercholesterolemia, diabetes mellitus, and hyperhomocysteinemia.

7. The method of claim 1 wherein said oxidative stress disorder is induced by ionizing radiation.

8. The method of claim 1 wherein said oxidative stress disorder is induced by chemotherapy.

9. The method of claim 1 wherein said oxidative stress disorder is induced by a combination of chemotherapy and ionizing radiation.

10. A method of treating oxidative stress in a mammal comprising administering to the mammal a pharmaceutical composition that comprises a telomere homolog oligonucleotide.

11. The method of claim 10 wherein the oligonucleotide is an oligonucleotide with at least 33% sequence identity to (TTAGGG)ₙ, wherein n is a number from 1 to 333.

12. The method of claim 11 wherein the sequence identity is at least 50%.

13. The method of claim 10 wherein the oligonucleotide is selected from the group consisting of GAGTATGAG (SEQ ID NO: 5), GTTAGGGTTAG (SEQ ID NO: 6), GGGTTAGGGTT (SEQ ID NO: 7), TAGATGTGGTG (SEQ ID NO: 8) and TT, said oligonucleotide optionally comprising a 5′-phosphate.

14. The method of claim 10 wherein the mammal is a human.

15. The method of claim 10 wherein said oxidative stress is induced by ionizing radiation.

16. The method of claim 10 wherein said oxidative stress is induced by chemotherapy.

17. The method of claim 10 wherein said oxidative stress is induced by a combination of chemotherapy and ionizing radiation.

18. A method of preventing an oxidative stress disorder in a mammal comprising administering to the mammal a pharmaceutical composition that comprises a telomere homolog oligonucleotide prior to or after induction of oxidative stress but prior to onset of the oxidative stress disorder.

19. The method of claim 18 wherein the oligonucleotide is an oligonucleotide with at least 33% sequence identity to (TTAGGG)ₙ, wherein n is a number from 1 to 333.

20. The method of claim 19 wherein the sequence identity is at least 50%.

21. The method of claim 18 wherein the oligonucleotide is selected from the group consisting of GAGTATGAG (SEQ ID NO: 5), GTTAGGGTTAG (SEQ ID NO: 6), GGGTTAGGGTT (SEQ ID NO: 7), TAGATGTGGTG (SEQ ID NO: 8) and TT, said oligonucleotide optionally comprising a 5′-phosphate.

22. The method of claim 18 wherein the mammal is a human.

23. The method of claim 18 wherein the oxidative stress disorder is selected from the group consisting of retinal degeneration, Alzheimer's disease, aging, skin photoaging, cardiovascular disease, hypertension, hypercholesterolemia, diabetes mellitus, and hyperhomocysteinemia.

24. The method of claim 18 wherein said oxidative stress disorder is induced by ionizing radiation.

25. The method of claim 18 wherein said oxidative stress disorder is induced by chemotherapy.

26. The method of claim 18 wherein said oxidative stress disorder is induced by a combination of chemotherapy and ionizing radiation.

27. A method of treating or preventing an oxidative stress disorder in a mammal comprising administering to the mammal a pharmaceutical composition comprising one or more oligonucleotides, said oligonucleotide having between 2 and 200 bases and having at least 33% but less than 100% identity with the sequence (TTAGGG)ₙ and optionally having a 5′-phosphate, and when said oligonucleotide comprises the sequence 5′-RRRGGG-3′ (R=any nucleotide) said oligonucleotide has a guanine content of 50% or less.

28. The method of claim 27, wherein said oligonucleotide lacks cytosine.

29. The method of claim 27, wherein said oligonucleotide comprises one or more sequences selected from the group consisting of TT, TA, TG, AG, GG, AT, GT, TTA, TAG, TAT, ATG, AGT, AGG, GAG, GGG, TTAG, TAGG, AGGG, GMT, GTTA, TTAGG, TAGGG,GGTTA, GTTAG, GGGTT and GGGGTT.

30. The method of claim 27, wherein said oligonucleotide is between 40% and 90% identical to (TTAGGG)ₙ.

31. The method of claim 27, wherein said oligonucleotide is selected from the group consisting of oligonucleotides 2-200 nucleotides long; oligonucleotides 2-20 nucleotides long; oligonucleotides 5-16 nucleotides long; and oligonucleotides 2-5 nucleotides long.

32. The method according to claim 27 wherein said one or more oligonucleotide is selected from the group consisting of: GTTAGGGTGTAGGTTT (SEQ ID NO: 9); GGTTGGTTGGTTGGTT (SEQ ID NO: 10); GGTGGTGGTGGTGGT (SEQ ID NO: 11); GGAGGAGGAGGAGGA (SEQ ID NO: 12); GGTGTGGTGTGGTGT (SEQ ID NO: 13); TAGTGTTAGGTGTAG (SEQ ID NO: 14); GAGTATGAG (SEQ ID NO: 5); AGTATGA; GGTTAGGGTTAG (SEQ ID NO: 6); GGTAGGTGTAGGATT (SEQ ID NO: 15); GGTAGGTGTAGGTTA (SEQ ID NO: 16); GGTTAGGTGTAGGTT (SEQ ID NO: 17); GGTTAGGTGGAGGTTT (SEQ ID NO: 18); GGTTAGGTTAGGTTA (SEQ ID NO: 19); GTTAGGTTTAAGGTT (SEQ ID NO: 20); and GTTAGGGTTAGGGTT (SEQ ID NO: 21).

33. The method of claim 27 wherein the mammal is a human.

34. The method of claim 27 wherein the oxidative stress disorder is selected from the group consisting of retinal degeneration, Alzheimer's disease, aging, skin photoaging, cardiovascular disease, hypertension, hypercholesterolemia, diabetes mellitus, and hyperhomocysteinemia.

35. The method of claim 27 wherein said oxidative stress disorder is induced by ionizing radiation.

36. The method of claim 27 wherein said oxidative stress disorder is induced by chemotherapy.

37. The method of claim 27 wherein said oxidative stress disorder is induced by a combination of chemotherapy and ionizing radiation.

38. A method of treating or preventing photoaging in a mammal comprising administering to the mammal a cosmetic composition that comprises a telomere homolog oligonucleotide.

39. The method of claim 38 wherein the oligonucleotide has at least 33% sequence identity to (TTAGGG)ₙ, wherein n is a number from 1 to 333.

40. The method of claim 39 wherein the sequence identity is at least 50%.

41. The method of claim 38 wherein the oligonucleotide is selected from the group consisting of GAGTATGAG (SEQ ID NO: 5), GTTAGGGTTAG (SEQ ID NO: 6), GGGTTAGGGTT (SEQ ID NO: 7), TAGATGTGGTG (SEQ ID NO: 8) and TT, said oligonucleotide optionally comprising a 5′-phosphate.

42. The method of claim 38 wherein the mammal is a human.

43. The method of claim 38 wherein said cosmetic composition comprises one or more oligonucleotides, said oligonucleotide having between 2 and 200 bases and having at least 33% but less than 100% identity with the sequence (TTAGGG)ₙ, and optionally having a 5′-phosphate, and when said oligonucleotide comprises the sequence 5′-RRRGGG-3′ (R=any nucleotide) said oligonucleotide has a guanine content of 50% or less.

44. The method of claim 38, wherein said oligonucleotide lacks cytosine.

45. The method of claim 38, wherein said oligonucleotide comprises one or more sequences selected from the group consisting of TT, TA, TG, AG, GG, AT, GT, TTA, TAG, TAT, ATG, AGT, AGG, GAG, GGG, TTAG, TAGG, AGGG, GGTT, GTTA, TTAGG, TAGGG,GGTTA, GTTAG, GGGTT and GGGGTT.

46. The method of claim 38, wherein said oligonucleotide is between 40% and 90% identical to (TTAGGG)ₙ.

47. The method of claim 38, wherein said oligonucleotide is selected from the group consisting of oligonucleotides 2-200 nucleotides long; oligonucleotides 2-20 nucleotides long; oligonucleotides 5-16 nucleotides long; and oligonucleotides 2-5 nucleotides long.

48. The method according to claim 38 wherein said one or more oligonucleotide is selected from the group consisting of: GTTAGGGTGTAGGTTT (SEQ ID NO: 9); GGTTGGTTGGTTGGTT (SEQ ID NO: 10); GGTGGTGGTGGTGGT (SEQ ID NO: 11); GGAGGAGGAGGAGGA (SEQ ID NO: 12); GGTGTGGTGTGGTGT (SEQ ID NO: 13); TAGTGTTAGGTGTAG (SEQ ID NO: 14); GAGTATGAG (SEQ ID NO: 5); AGTATGA; GTTAGGGTTAG (SEQ ID NO: 6); GGTAGGTGTAGGATT (SEQ ID NO: 15); GGTAGGTGTAGGTTA (SEQ ID NO: 16); GGTTAGGTGTAGGTT (SEQ ID NO: 17); GGTTAGGTGGAGGTTT (SEQ ID NO: 18); GGTTAGGTTAGGTTA (SEQ ID NO: 19); GTTAGGTTTAAGGTT (SEQ ID NO: 20); and GTTAGGGTTAGGGTT (SEQ ID NO: 21).


SEQUENCE LISTING (27 sequences)

SEQ ID NO: 1 - DNA, 11 bp, Artificial
  Synthetic oligonucleotide
gttagggttag

SEQ ID NO: 2 - DNA, 9 bp, Artificial
  Synthetic oligonucleotide
gagtatgag

SEQ ID NO: 3 - DNA, 11 bp, Artificial
  Synthetic oligonucleotide
gggttagggtt

SEQ ID NO: 4 - DNA, 11 bp, Artificial
  Synthetic oligonucleotide
tagatgtggtg

SEQ ID NO: 5 - DNA, 9 bp, Artificial
  Synthetic oligonucleotide
gagtatgag

SEQ ID NO: 6 - DNA, 11 bp, Artificial
  Synthetic oligonucleotide
gttagggttag

SEQ ID NO: 7 - DNA, 11 bp, Artificial
  Synthetic oligonucleotide
gggttagggtt

SEQ ID NO: 8 - DNA, 11 bp, Artificial
  Synthetic oligonucleotide
tagatgtggtg

SEQ ID NO: 9 - DNA, 16 bp, Artificial
  Synthetic oligonucleotide
gttagggtgtaggttt

SEQ ID NO: 10 - DNA, 16 bp, Artificial
  Synthetic oligonucleotide
ggttggttggttggtt

SEQ ID NO: 11 - DNA, 15 bp, Artificial
  Synthetic oligonucleotide
ggtggtggtggtggt

SEQ ID NO: 12 - DNA, 15 bp, Artificial
  Synthetic oligonucleotide
ggaggaggaggagga

SEQ ID NO: 13 - DNA, 15 bp, Artificial
  Synthetic oligonucleotide
ggtgtggtgtggtgt

SEQ ID NO: 14 - DNA, 15 bp, Artificial
  Synthetic oligonucleotide
tagtgttaggtgtag

SEQ ID NO: 15 - DNA, 15 bp, Artificial
  Synthetic oligonucleotide
ggtaggtgtaggatt

SEQ ID NO: 16 - DNA, 15 bp, Artificial
  Synthetic oligonucleotide
ggtaggtgtaggtta

SEQ ID NO: 17 - DNA, 15 bp, Artificial
  Synthetic oligonucleotide
ggttaggtgtaggtt

SEQ ID NO: 18 - DNA, 16 bp, Artificial
  Synthetic oligonucleotide
ggttaggtggaggttt

SEQ ID NO: 19 - DNA, 15 bp, Artificial
  Synthetic oligonucleotide
ggttaggttaggtta

SEQ ID NO: 20 - DNA, 15 bp, Artificial
  Synthetic oligonucleotide
gttaggtttaaggtt

SEQ ID NO: 21 - DNA, 15 bp, Artificial
  Synthetic oligonucleotide
gttagggttagggtt

SEQ ID NO: 22 - DNA, 11 bp, Artificial
  Synthetic oligonucleotide
ctaaccctaac

SEQ ID NO: 23 - DNA, 11 bp, Artificial
  Synthetic oligonucleotide
gatcgatcgat

SEQ ID NO: 24 - DNA, 9 bp, Artificial
  Synthetic oligonucleotide
taggaggat

SEQ ID NO: 25 - DNA, 9 bp, Artificial
  Synthetic oligonucleotide
taggaggat

SEQ ID NO: 26 - DNA, 21 bp, Artificial
  Synthetic oligonucleotide
ctacttatcgagaatgtggcg

SEQ ID NO: 27 - DNA, 20 bp, Artificial
  Synthetic oligonucleotide
cgatgtcaatggtctggaag